(19) United States
(12) Patent Application Publication
(10) Pub. No.: US 2024/0002333 A1
(43) Pub. Date: Jan. 4, 2024

(54) LINEAR AND CYCLIC AROMATIC OLIGOAMIDES, METHODS OF MAKING SAME, AND USES THEREOF

(71) Applicant: The Research Foundation for The State University of New York, Amherst, NY (US)

(72) Inventor: Bing Gong, Amherst, NY (US)

(21) Appl. No.: 18/044,977

(22) PCT Filed: Sep. 13, 2021
(86) PCT No.: PCT/US21/50041

§ 371 Date: Mar. 10, 2023

Related U.S. Application Data
(60) Provisional application No. 63/077,696, filed on Sep. 13, 2020.

(51) Int. Cl. C07C 237/42; C02F 1/68

(52) U.S. Cl. CPC C07C 237/42; C02F 1/683

(57) ABSTRACT

The present disclosure provides linear and cyclic oligoamides. The present disclosure also provides methods of making and uses of linear and cyclic oligoamides. The linear and/or cyclic oliogoamides may be used in methods such as, for example, forming transmembrane pores for transmembrane transport of hydrogen-bond acceptors and/or ions, sequestering hydrogen-bond acceptors and/or ions (e.g., anions), or the like. Compounds of the present disclosure may be used to enrich materials with ions (e.g., lithium). Compounds of the present disclosure may have the following structure: Structure (I) or Structure (II)

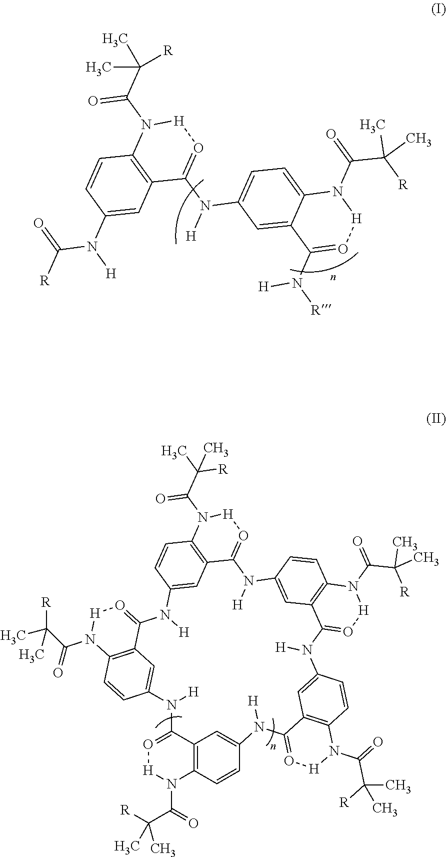

### CROSS REFERENCE TO RELATED APPLICATIONS

This application claims priority to U.S. Provisional Application No. 63/077,696, filed on Sep. 13, 2020, the disclosure of which is incorporated herein by reference.

### STATEMENT REGARDING FEDERALLY SPONSORED RESEARCH

This invention was made with government support under contract no. CHE-1905094 awarded by the National Science Foundation. The government has certain rights in the invention.

### BACKGROUND OF THE DISCLOSURE

Anion recognition, as a major theme of supramolecular chemistry, is attracting wide attention in a variety of areas because of the central role played by anions in biology. Anions are found in the anionic centers of most biomolecules. The majority of enzyme substrates are anionic. The maintenance of cellular functions relies on transmembrane anion transport. The development of anion binders and receptors has great potential for various biological activities. Many diseases are related to ion transport and anion channels. By promoting transmembrane transport of anions, either through anion carriers or anion channels, genetic conditions and diseases caused by defective anion channels could be remedied. For example, cystic fibrosis, a genetic disorder caused by a mutation in the gene cystic fibrosis transmembrane conductance regulator (CFTR) and disfunctioning chloride channel, is accompanied by long-term complications including difficulty breathing and coughing up mucus as a result of frequent lung infections. Other signs and symptoms related to malfunctioned channels may include sinus infections, poor growth, fatty stool, clubbing of the fingers and toes, and infertility in most males. Although there is no known cure for cystic fibrosis, synthetic anion carriers and channels could substitute the mutated CFTR chloride channels and restore transmembrane chloride conductance, which alleviates the symptoms of cystic fibrosis. For this reason, anion carriers and channels especially those for the chloride ion are being actively pursued. In comparison to the numerous synthetic cation binders and receptors known thus far, much less anion receptors have been reported. The design of anion binders or receptors typically requires the convergent arrangement of multiple hydrogen-bond donors which engage in multiple cooperative H-bonding interactions with an anion guest. The H-bond donors are based on the NH groups of amide, urea, or thiourea groups. However, due to the inherent structural limitation of oligoamides and oligoureas, arranging multiple NH groups convergently turns out to be a rather challenging task. As a result, the convergent placement of multiple NH groups has to rely on cyclic structures, that may not be readily available synthetically. Besides, few examples of anion binders with systematically variable binding strength are known.

### SUMMARY OF THE DISCLOSURE

The present disclosure provides linear and cyclic oligoamides. The present disclosure also provides methods of making and uses of linear and cyclic oligoamides.

In an aspect, the present disclosure provides compounds. The compounds are linear or cyclic oligoamides. A compound, which may be a linear or cyclic macrocyclic oliogamide (which may also be referred to as a macrocyclic compound/oligoamide), may comprise one or more aromatic substituents. In the case where a compound comprises a plurality of aromatic substituents, adjacent aromatic substituents are linked by at least one amide group. Non-limiting examples of linear and cyclic/macrocyclic oligoamides are provided herein.

A linear oligoamide or cyclic oligoamide may a curved backbone. Not intending to be bound by any particular theory, the curved backbone is largely due to intramolecular hydrogen bonds that rigidify the amide linkage of each amide group to each aromatic substituent and at least in part to an interaction between the aromatic substituents (e.g., π-π interactions), whereby the curved backbone is stabilized.

In various examples, a linear compound of the present disclosure has the following structure:

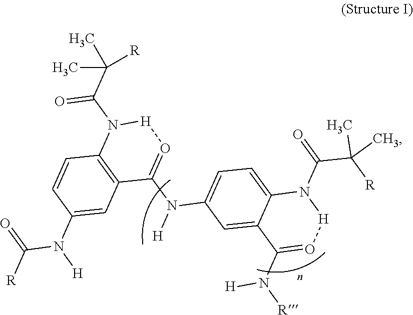

where n is 0 to 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50). R is independently at each occurrence chosen from linear aliphatic groups (which may be C₁-C₂₀ linear aliphatic groups (e.g., linear alkyl groups, such as, for example, methyl, ethyl, propyl, butyl, pentyl, hexyl, heptyl, octyl groups, and the like, which may be C₁-C₂₀ linear alkyl groups)); branched aliphatic groups (which may be C₁-C₂₀ branched aliphatic groups (e.g., branched alkyl groups, such as, for example, isopropyl, isobutyl, t-butyl, neopentyl, isopentyl groups, and the like, which may be C₁-C₂₀ branched alkyl groups)); fluorinated linear aliphatic groups (which may be C₁-C₂₀ fluorinated linear aliphatic groups (e.g., fluorinated linear alkyl groups, such as, for example, fluoromethyl, fluoroethyl, fluoropropyl, fluorobutyl, fluoropentyl, fluorohexyl, fluoroheptyl, fluorooctyl groups, and the like), which comprise one or more fluorine group(s) (e.g., a fluorinated linear aliphatic group (such as, for example, a fluorinated linear alkyl group or the like) is a perfluorinated linear aliphatic group (e.g., alkyl and the like))); fluorinated branched aliphatic groups (which may be C₁-C₂₀ fluorinated branched alkyl groups (e.g., fluorinated branched alkyl groups, such as, for example, branched derivatives of fluoropropyl, fluorobutyl, fluoropentyl, fluorohexyl, fluoroheptyl, fluorooctyl groups, and the like) and the like, which comprise one or more fluorine group(s) (e.g., a fluorinated branched aliphatic group (such as, for example, a fluorinated branched alkyl group or the like) is a perfluorinated branched aliphatic group (e.g., alkyl or the like))); ether groups (which may comprise one or two group(s) chosen from linear aliphatic groups (e.g., linear alkyl groups and the like), branched aliphatic groups (e.g., branched alkyl groups and the like), fluorinated linear aliphatic groups (e.g., fluorinated linear alkyl groups and the like), fluorinated branched aliphatic groups (e.g., fluourinated branched alkyl groups and the like) (e.g., —(CH₂)₂OCH₃, —(CH₂)₂OCH₂CH₃, —(CH₂)₂OCH₂CH(CH₃)₂, and —(CH₂)₂O(CH₂)₂CH(CH₃)₂, fluorinated analogs thereof, and the like)); and oligoether groups (which may comprise one or more group(s) chosen linear aliphatic groups (e.g., linear alkyl groups and the like), branched aliphatic groups (e.g., branched alkyl groups and the like), fluorinated linear aliphatic groups (e.g., fluorinated linear alkyl groups and the like) (e.g., ethyl groups, propyl groups, and the like and combinations thereof and/or may comprise one or more fluorine groups (e.g., the ether group may be perfluorinated)) (e.g.,

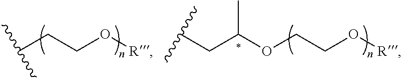

fluorinated analogs thereof, and the like, where the asterisk denotes a stereogenic carbon (i.e., a carbon having R or S stereochemistry), n is 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 8, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100), and R′″ is a linear or branched aliphatic group (e.g., alkyl group and the like) (e.g., methyl, ethyl, propyl, isopropyl, butyl, isobutyl, t-butyl, pentyl, neopentyl, isopentyl, and the like), which may comprise one or more fluorine groups (e.g., the oligoether group may be perfluorinated)), and combinations thereof.

In various examples, when the linear compound has structure I, n is 0, 1, 2, 3, or 4; R′ is chosen from methyl, ethyl, propyl, butyl, pentyl, hexyl, heptyl, octyl, and nonyl; R″ is chosen from methyl, ethyl, propyl, butyl, pentyl, and hexyl; R is —C(CH₃)₂CH₂O(CH₂)₇CH₃.

In various examples, a compound of the present disclosure has the following structure:

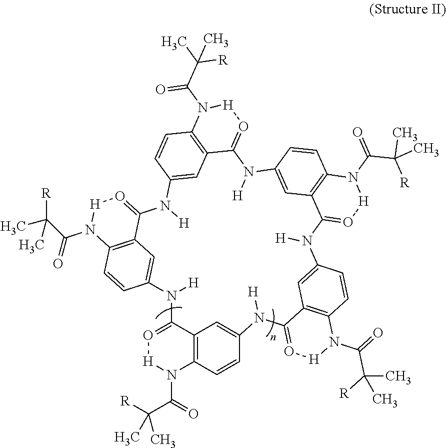

where n is 1 or 2. R is independently at each occurrence chosen from linear aliphatic groups (which may be C₁-C₂₀ linear aliphatic groups (e.g., linear alkyl groups, such as, for example, methyl, ethyl, propyl, butyl, pentyl, hexyl, heptyl, octyl groups, and the like, which may be C₁-C₂₀ linear alkyl groups)); branched aliphatic groups (which may be C₁-C₂₀ branched aliphatic groups (e.g., branched alkyl groups, such as, for example, isopropyl, isobutyl, t-butyl, neopentyl, isopentyl groups, and the like, which may be C₁-C₂₀ branched alkyl groups)); fluorinated linear aliphatic groups (which may be C₁-C₂₀ fluorinated linear aliphatic groups (e.g., fluorinated linear alkyl groups, such as, for example, fluoromethyl, fluoroethyl, fluoropropyl, fluorobutyl, fluoropentyl, fluorohexyl, fluoroheptyl, fluorooctyl groups, and the like), which comprise one or more fluorine group(s) (e.g., a fluorinated linear aliphatic group (such as, for example, a fluorinated linear alkyl group or the like) is a perfluorinated linear aliphatic group (e.g., alkyl and the like))); fluorinated branched aliphatic groups (which may be C₁-C₂₀ fluorinated branched alkyl groups (e.g., fluorinated branched alkyl groups, such as, for example, branched derivatives of fluoropropyl, fluorobutyl, fluoropentyl, fluorohexyl, fluoroheptyl, fluorooctyl groups, and the like) and the like, which comprise one or more fluorine group(s) (e.g., a fluorinated branched aliphatic group (such as, for example, a fluorinated branched alkyl group or the like) is a perfluorinated branched aliphatic group (e.g., alkyl or the like))); ether groups (which may comprise one or two group(s) chosen from linear aliphatic groups (e.g., linear alkyl groups and the like), branched aliphatic groups (e.g., branched alkyl groups and the like), fluorinated linear aliphatic groups (e.g., fluorinated linear alkyl groups and the like), fluorinated branched aliphatic groups (e.g., fluourinated branched alkyl groups and the like) (e.g., —(CH₂)₂OCH₃, —(CH₂)₂OCH₂CH₃, —(CH₂)₂OCH₂CH(CH₃)₂, and —(CH₂)₂O(CH₂)₂CH(CH₃)₂, fluorinated analogs thereof, and the like)); and oligoether groups (which may comprise one or more group(s) chosen linear aliphatic groups (e.g., linear alkyl groups and the like), branched aliphatic groups (e.g., branched alkyl groups and the like), fluorinated linear aliphatic groups (e.g., fluorinated linear alkyl groups and the like) (e.g., ethyl groups, propyl groups, and the like and combinations thereof and/or may comprise one or more fluorine groups (e.g., the ether group may be perfluorinated)) (e.g.,

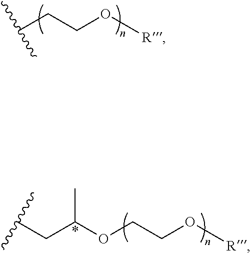

fluorinated analogs thereof, and the like, where the asterisk denotes a stereogenic carbon (i.e., a carbon having R or S stereochemistry), n is 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 8, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100), and R′″ is a linear or branched aliphatic group (e.g., alkyl group and the like) (e.g., methyl, ethyl, propyl, isopropyl, butyl, isobutyl, t-butyl, pentyl, neopentyl, isopentyl, and the like), which may comprise one or more fluorine groups (e.g., the oligoether group may be perfluorinated)), and combinations thereof.

In an aspect, the present disclosure provides compositions. A composition may comprise one or more linear and/or one or more cyclic oligoamide of the present disclosure. A composition may be a pharmaceutical composition. A composition may be suitable for administration to an individual. Non-limiting examples of compositions are provided herein.

In an aspect, the present disclosure provides method of making linear and cyclic oligoamides. In various examples, a linear or cyclic amide of the present disclosure is made by a method of the present disclosure. Non-limiting examples of methods of making linear and cyclic oligoamides are provided herein.

In an aspect, the present disclosure provides uses of linear and cyclic oliogoamides. The linear and/or cyclic oliogoamides may be used in methods such as, for example, forming transmembrane pores for transmembrane transport of hydrogen-bond acceptors and/or ions, sequestering hydrogen-bond acceptors and/or ions (e.g., anions), or the like. Compounds of the present disclosure may be used to enrich materials with ions (e.g., lithium). Non-limiting examples of uses of linear and/or cyclic oligoamides are provided herein.

### BRIEF DESCRIPTION OF THE FIGURES

For a fuller understanding of the nature and objects of the disclosure, reference should be made to the following detailed description taken in conjunction with the accompanying figures.

FIG. 1 shows (a) previously developed aromatic oligoamide foldamer A has a fully constrained backbone. Oligoamide H, with the orientation of its backbone amide groups (red) being inverted relative to those of A, has multiple amide NH groups available for H-bonding. (b) Instead of adopting multiple conformations as represented by H′, oligoamide H, upon binding a guest via H-bonding, will be driven to adopt a crescent conformation in which all backbone amide protons and “internal” aromatic protons, are convergently arranged.

FIG. 2 shows a reactions scheme for the conversion of B1 to B2.

FIG. 3 shows (a) X-ray structure of compound B2b. (b) X-ray structure of compound 2b′ reveal two conformations related by rotation around the single bonds as indicated by arrows.

FIG. 4 shows (a) Two outcomes for the ring-opening reactions of (4H)-3, 1-benzoxazine-4-one C with a nucleophile such as an amine. (b) Treating n-octylamine with compounds B2a and B2b, respectively, gives products B3 and 1b.

FIG. 5 shows a reaction scheme of (a) 1b, 1c to 1-5 and (b) 2c′ and 4c-NH₂ to form 6c.

FIG. 6 shows Partial NOESY spectra of (a) 4c (5 mM) and (b) 4c (5 mM) and tetrabutylammonium iodide (5 mM) in CDCl₃ containing 5% DMSO-d₆ (500 MHz, 25° C., mixing time=0.4 s). The observed NOEs are indicated by double-headed arrows.

FIG. 7 shows (left) a reaction scheme of the precursors to a compound of the present disclosure and (right) a schematic depicting the binding of a polar species such as an anion or a negatively charged group.

FIG. 8 shows ¹H NMR spectra of B2a, B2b, and B2c. (400 MHz, 25° C., 10 mM of corresponding benzoxazinone derivatives in CDCl₃).

FIG. 9 shows mass spectra (ESI-MS) of B2a (m/z [M+H]⁺ Calcd for C₁₇H₂₃N₂O₄ 319.37; Found 319.24), B2b (m/z [M+H]⁺ Calcd for C₁₂H₁₃N₂O₄ 249.24; Found 249.06), and B2c (m/z [M+H]⁺ Calcd for C₂₀H₂₉N₂O₅ 377.45; Found 377.12). Acetonitrile was used as the mobile phase, since benzoxazinone derivatives (B2a, B2b, and B2c) could undergo the ring-opening reaction with methanol and give corresponding methyl esters in the presence of methanol.

FIG. 10 shows mass spectra (ESI-MS) of 1b (m/z [M+H]⁺ Calcd for C₂₀H₃₂N₃O₄ 378.48; Found 378.11; m/z [M+Na]⁺ Calcd for C₂₀H₃₁N₃NaO₄ 400.46; Found 400.18) and B3 (m/z [M+H]*Calcd for C₂₅H₄₀N₃O₃ 430.60; Found 430.36). Methanol was used as the mobile phase.

FIG. 11 shows ¹H NMR spectra of 1b and B3. (400 MHz, 25° C., 20 mM of corresponding materials in CDCl₃).

FIG. 12 shows ¹H NMR spectra of 2b′ and 2c′. (400 MHz, 25° C., 15 mM of corresponding materials in CDCl₃).

FIG. 13 shows partial stacked ¹H NMR spectra of 4c with 0.0 to 2.0 equiv of TBACl (top) and TBAI (bottom) (400 MHz, 25° C., 5 mM of 4c in 10% CD₃CN/90% CDCl₃. The peak of TMS was set to 0.0 ppm. The chemical shift of corresponding amide protons are shown with dash lines).

FIG. 14 shows partial stacked ¹H NMR spectra of 6c with 0.0 to 2.0 equiv of TBACl (top) and TBAI (bottom) (400 MHz, 25° C., 2 mM of 6c in 5% DMSO-d₆/95% CDCl₃. The peak of TMS was set to 0.0 ppm. The chemical shifts of corresponding amide protons are shown with dashed lines).

FIG. 15 shows partial stacked ¹H NMR spectra of 4c with 0 to 2 equiv of TBAPh₄B (400 MHz, 25° C., 5 mM of 4c in 10% CD₃CN/90% CDCl₃. The peak of TMS was set to 0.0 ppm. The chemical shift of corresponding amide protons are shown with dashed lines).

FIG. 16 shows NOESY of 4c showing NOE interaction between protons d and 4. (500 MHz, 25° C., 5 mM of 4c in 5% DMSO-d₆/95% CDCl₃, mixing time=300 ms).

FIG. 17 shows NOESY spectrum of 4c with 1 equiv of TBAI showing NOE interaction between protons d and 4. (500 MHz, 25° C., 5 mM in 5% DMSO-d₆/95% CDCl₃, mixing time=300 ms).

FIG. 18 shows NOESY spectrum of 4c with 1 equiv of TBACl showing NOE interaction between amide NH protons a, b, c and aromatic CH protons 1, 2, 3, 4. (500 MHz, 25° C., 5 mM in 5% DMSO-d₆/95% CDCl₃, mixing time=300 ms).

FIG. 19 shows partial NOESY spectra of 6c (2 mM) showing NOE interactions between amide NH protons a, b, e, f and aromatic CH protons 1, 2, 3, 5, 6. (500 MHz, 25° C., 5% DMSO-d₆/95% CDCl₃, mixing time=300 ms). The NH protons c and d are overlapped and their NOE interactions with aromatic CH protons cannot be assigned accurately. The important NOE interactions were shown with double-head arrows.

FIG. 20 shows labeled partial ¹H NMR spectra of 6c and equimolar amount of TBACl. (500 MHz, 25° C., 2 mM in the 5% DMSO-d₆/95% CDCl₃).

FIG. 21 shows partial NOESY spectra of 6c with equimolar amount of TBACl showing NOE interaction between amide NH protons a, b, c, d, e, f and aromatic CH protons 1, 2, 3, 4, 5, 6. (500 MHz, 25° C., 2 mM in 5% DMSO-d₆/95% CDCl₃, mixing time=300 ms). The important NOE interactions were shown with double-head arrows.

FIG. 22 shows partial ¹H NMR of 4c. (500 MHz, 25° C., 5 mM in the 5% DMSO-d₆/95% CDCl₃).

FIG. 23 shows partial 2-D gCOSY of 4c. (500 MHz, 25° C., 5 mM in the 5% DMSO-d₆/95% CDCl₃, mixing time=300 ms, and interactions highlighted with double-head arrows).

FIG. 24 shows partial ¹H NMR of 1:1 complex of 4c TBACl. (500 MHz, 25° C., 5 mM in the 5% DMSO-d₆/95% CDCl₃).

FIG. 25 shows partial 2-D gCOSY of 1:1 complex of 4c TBACl. (500 MHz, 25° C., 5 mM in the 5% DMSO-d₆/95% CDCl₃, mixing time=300 ms and interactions highlighted with double-head arrows).

FIG. 26 shows partial ¹H-NMR of 1:1 complex of 4c TBAI. (500 MHz, 5 mM in the 5% DMSO-d₆/95% CDCl₃).

FIG. 27 shows partial 2-D gCOSY of 1:1 complex of 4c TBAI. (500 MHz, 5 mM in the 5% DMSO-d₆/95% CDCl₃, 25° C., mixing time=300 ms, and interactions highlighted with double-head arrows).

FIG. 28 shows partial ¹H NMR of 4c (400 MHz, 4 mM in different mixed solvents, 25° C.). The amide protons of interest were shown using dashed lines. The peak of TMS was set to 0.0 ppm. In ¹H NMR of 4c in pure DMSO-d₆, the DMSO-d₆ residue was set to 2.49 ppm. In ¹H NMR of 4c in pure CDCl₃, the proton peaks showed significant line broadening and no accurate assignment was obtained.

FIG. 29 shows partial ¹H NMR of 6c. (500 MHz, 25° C., 2 mM in the 5% DMSO-d₆/95% CDCl₃).

FIG. 30 shows chemical shift of amide NH protons of 4c with increasing amount of TBACl (top, Ka=860±40 M⁻¹) and TBAI (bottom, Ka=400±10 M⁻¹) and corresponding fitting output from BindFit v0.5. (5 mM of 4c in 10% CD₃CN/90% CDCl₃, 25° C., 400 MHz).

FIG. 31 shows chemical shift of amide NH protons of 6c with increasing amount of TBACl (top, Ka=2240±250 M⁻¹) and TBAI (bottom, Ka=2340±120 M⁻¹) and corresponding fitting output from Bindfit v0.5. (2 mM of 6c in 5% DMSO-d₆/95% CDCl₃, 25° C., 400 MHz).

FIG. 32 shows ¹H NMR of compound 1 (CDCl₃, 25° C., 500 MHz).

FIG. 33 shows ¹³C NMR of compound 1 (CDCl₃, 25° C., 75 MHz).

FIG. 34 shows HRMS-ESI spectrum of compound 1 (positive mode).

FIG. 35 shows ¹H NMR of compound 2 (CDCl₃, 25° C., 400 MHz).

FIG. 36 shows ¹³C NMR of compound 2 (CDCl₃, 25° C., 75 MHz).

FIG. 37 shows HRMS-ESI spectrum of compound 2 (positive mode).

FIG. 38 shows ¹H NMR of compound B2a (CDCl₃, 25° C., 400 MHz).

FIG. 39 shows ¹³C NMR of compound B2a (CDCl₃, 25° C., 75 MHz).

FIG. 40 shows ¹H NMR of compound B2b (CDCl₃, 25° C., 400 MHz).

FIG. 41 shows ¹³C NMR of compound B2b (CDCl₃, 25° C., 75 MHz).

FIG. 42 shows ¹H NMR of compound B2c (CDCl₃, 25° C., 400 MHz).

FIG. 43 shows ¹³C NMR of compound B2c (CDCl₃, 25° C., 75 MHz).

FIG. 44 shows ¹H NMR of compound B3 (CDCl₃, 25° C., 400 MHz).

FIG. 45 shows ¹³C NMR of compound B3 (CDCl₃, 25° C., 75 MHz).

FIG. 46 shows HRMS-ESI spectrum of compound B3 (positive mode).

FIG. 47 shows ¹HNMR of compound 1b (CDCl₃, 25° C., 500 MHz).

FIG. 48 shows ¹³C NMR of compound 1b (CDCl₃, 25° C., 75 MHz).

FIG. 49 shows HRMS-ESI spectrum of compound 1b (positive mode).

FIG. 50 shows ¹H NMR of compound 1c (CDCl₃, 25° C., 400 MHz).

FIG. 51 shows ¹³C NMR of compound 1c (CDCl₃, 25° C., 75 MHz).

FIG. 52 shows HRMS-ESI spectrum of compound 1c (positive mode).

FIG. 53 shows ¹H NMR of compound 7b (CDCl₃, 25° C., 500 MHz).

FIG. 54 shows ¹³C NMR of compound 7b (DMSO-d₆, 25° C., 75 MHz).

FIG. 55 shows HRMS-ESI spectrum of compound 7b (positive mode).

FIG. 56 shows ¹H NMR of compound 7c (CDCl₃, 25° C., 400 MHz).

FIG. 57 shows ¹³C NMR of compound 7c (CDCl₃, 25° C., 75 MHz).

FIG. 58 shows HRMS-ESI spectrum of compound 7c (positive mode).

FIG. 59 shows ¹H NMR of compound 8b (CDCl₃, 25° C., 500 MHz).

FIG. 60 shows ¹³C NMR of compound 8b (DMSO-d₆, 25° C., 75 MHz).

FIG. 61 shows HRMS-ESI spectrum of compound 8b (positive mode).

FIG. 62 shows ¹H NMR of compound 8c (CDCl₃, 25° C., 400 MHz).

FIG. 63 shows ¹³C NMR of compound 8c (CDCl₃, 25° C., 75 MHz).

FIG. 64 shows HRMS-ESI spectrum of compound 8c (positive mode).

FIG. 65 shows ¹H NMR of compound 9b (CDCl₃, 25° C., 500 MHz).

FIG. 66 shows ¹³C NMR of compound 9b (DMSO-d₆, 25° C., 75 MHz).

FIG. 67 shows HRMS-ESI spectrum of compound 9b (positive mode).

FIG. 68 shows ¹H NMR of compound 9c (CDCl₃, 25° C., 400 MHz).

FIG. 69 shows ¹³C NMR of compound 9c (CDCl₃, 25° C., 75 MHz).

FIG. 70 shows HRMS-ESI spectrum of compound 9c (positive mode).

FIG. 71 shows ¹H NMR of compound 2b′ (CDCl₃, 25° C., 500 MHz).

FIG. 72 shows ¹³C NMR of compound 2b′ (DMSO-d₆, 25° C., 75 MHz).

FIG. 73 shows ¹H NMR of compound 2c′ (CDCl₃, 25° C., 400 MHz).

FIG. 74 shows ¹³C NMR of compound 2c′ (CDCl₃, 25° C., 75 MHz).

FIG. 75 shows ¹H NMR of compound 2b (CDCl₃, 25° C., 500 MHz).

FIG. 76 shows ¹³C NMR of compound 2b (CDCl₃, 25° C., 75 MHz).

FIG. 77 shows HRMS-ESI spectrum of compound 2b (positive mode).

FIG. 78 shows ¹H NMR of compound 2c (CDCl₃, 25° C., 400 MHz).

FIG. 79 shows ¹³C NMR of compound 2c (CDCl₃, 25° C., 75 MHz).

FIG. 80 shows HRMS-ESI spectrum of compound 2c (positive mode).

FIG. 81 shows ¹H NMR of compound 3b (CDCl₃, 25° C., 500 MHz).

FIG. 82 shows ¹³C NMR of compound 3b (CDCl₃, 25° C., 75 MHz).

FIG. 83 shows HRMS-ESI spectrum of compound 3b (positive mode).

FIG. 84 shows ¹H NMR of compound 3c (10% DMSO-d₆/90% CDCl₃, 25° C., 400 MHz).

FIG. 85 shows ¹³C NMR of compound 3c (10% DMSO-d₆/90% CDCl₃, 25° C., 75 MHz).

FIG. 86 shows HRMS-ESI spectrum of compound 3c (positive mode).

FIG. 87 shows ¹H NMR of compound 4c (10% DMSO-d₆/90% CDCl₃, 25° C., 400 MHz).

FIG. 88 shows ¹³C NMR of compound 4c (10% DMSO-d₆/90% CDCl₃, 25° C., 75 MHz).

FIG. 89 shows HRMS-ESI spectrum of compound 4c (positive mode).

FIG. 90 shows ¹H NMR of compound 5c (10% DMSO-d₆/90% CDCl₃, 25° C., 400 MHz).

FIG. 91 shows ¹³C NMR of compound 5c (10% DMSO-d₆/90% CDCl₃, 25° C., 75 MHz).

FIG. 92 shows HRMS-ESI spectrum of compound 5c (positive mode).

FIG. 93 shows ¹H NMR of compound 6c (10% DMSO-d₆/90% CDCl₃, 25° C., 400 MHz).

FIG. 94 shows ¹³C NMR of compound 6c (10% DMSO-d₆/90% CDCl₃, 25° C., 75 MHz).

FIG. 95 shows HRMS-MALDI spectrum of compound 6c (positive mode). In the HRMS-MALDI spectrum, the monomer fragment (m/z: 347.2344) is also observed, indicating that 6c is not stable in the HRMS-MALDI.

FIG. 96 shows a copy of a thermal ellipsoid plot for the crystal structure of B2b which was drawn at 30% probability.

FIG. 97 shows a copy of a thermal ellipsoid plot for the crystal structure of 2b′ which was drawn at 30% probability.

FIG. 98 shows (a) general structure of previously developed aromatic oligoamide foldamer AO with a backbone fully constrained by three-center intramolecular H-bonds. Inverting the orientation of the backbone amide groups of AO gives aromatic oligoamide OA. (b) The population of crescent conformation OA, in which all backbone amide protons and the “inner” aromatic protons are convergently arranged, will be increased upon binding to a guest (sphere) via H-bonding interactions (dashed lines).

FIG. 99 shows (a) design of aromatic oligoamide macrocycles cOA as cyclic anion binders. (b) Energy-minimized structure of the cyclic 5mer (R=methyl) with an inner cavity of ˜6.8 Å (van der Waals 4.4 Å) across.

FIG. 100 shows X-ray structure of compound A2b.

FIG. 101 (a) two outcomes for the ring-opening reactions of (4H)-3, 1-benzoxazine-4-one B with a nucleophile such as an amine. (b) Treating n-octylamine with compounds A2a and A2b, respectively, gives products C and 1b.

FIG. 102 shows a crystal structure of compound 2b′ reveals two conformations that are related by rotation around the amide-aryl bond indicated by arrows.

FIG. 103 shows binding constants Kₐ (M⁻¹) of amide hosts with anions. The titrations were performed in mixed solvent containing 10% CD₃CN in CDCl₃ at room temperature.

FIG. 104 shows binding constants Kₐ (M⁻¹) of 7c with anions.

FIG. 105 shows the general structure (top) and crystal structure (bottom) of a cyclic compound of the present disclosure. In the top image, R=Sidechain. The property of the sidechain determines the compatibility of the macrocycle (MC) and anion-MC complex(es) with different solvents. The bottom image shows the persistent shape of the macrocyclic backbone; and the rigidly held, multiple NH and CH hydrogen bond donors that orient toward the center of the inner cavity which is predisposed for binding anions

FIG. 106 shows a schematic of an anion binding to a cyclic compound of the present disclosure. Specifically, binding to the c5mer wraps an anion in a layer of “cloth”, making the anion compatible with (soluble in) a solvent (e.g., a hydrophobic one) in which a naked anion cannot go into. The shaded circle can represent an ion, such as, for example, chloride (Cl⁻), bromide (Br⁻), iodide (I⁻), or other anion. For example, the compound may have high affinity for the binding of anions. Binding constants (Kₐ)>10⁷ M⁻¹ for Cl⁻, Br⁻, I⁻ in chloroform (CHCl₃). Order of binding strength: Cl⁻<Br⁻<I⁻. Binding of environmentally important anions (Kₐ's measured in chloroform with 5% methanol): sulfate (SO₄²⁻): 4×10⁵ M⁻¹, perchlorate (ClO₄⁻): 3×10⁶ M⁻¹, and nitrate (NO₃⁻): 2×10⁶ M⁻¹.

FIG. 107 shows a schematic for assessment of the transport of halides across the cell membranes (e.g., lipid bilayers). Lipids (X⁻) are hydrophilic and cannot cross cell membranes. Upon being bound to c5mer, an anion is carried (shuttled) by the macrocyclc to cross the membrane and delivered into liposomes (or large unilamellar vesicles (LUVs)). The internalized anions (and protons), are detected by the quenched emission of HPTS.

FIG. 108 shows c5mer-mediated transport of halides across cell membranes and the results from vesicle (LUV)-based assays. The efficient and selective transport of the chloride ion across cell membranes by the c5mer suggests that the macrocycle could serve as an effective carrier to restore chloride transport to cells with defect anion transport, a hallmark of the genetic disease cystic fibrosis (CF).

FIG. 109 shows the transport of anions (via ion pairs) across bulk organic phase. Organic cations have been used as the counterions to assist the transport of anions (e.g., Cl⁻) across the organic phase.

FIG. 110 shows enrichment of lithium ions with liquid membranes. The top layer is a solution (mixture) of LiCl, NaCl, and KCl in water. The middle layer shows a liquid membrane (LM). The solubilization of LiCl into the LM, assisted by an organic anion binder (e.g., the c5mer) for chloride and an organic binder specific for the lithium ion. The bottom layer shows the second (or n^{th}) aqueous phase. LiCl is release from the LM into the second aqueous phase.

FIG. 111 shows the general structure of cyclic compound of the present disclosure and possible synthetic routes.

FIG. 112 shows a synthetic route for a cyclic compound of the present disclosure with six repeat units.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

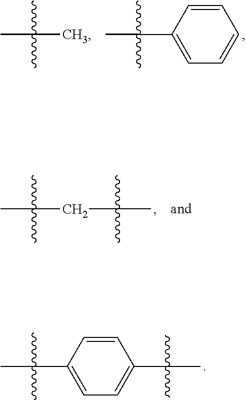

As used herein, unless otherwise indicated, the term “aliphatic groups” refers to branched or unbranched hydrocarbon groups that, optionally, contain one or more degrees of unsaturation. Degrees of unsaturation include, but are not limited to, alkenyl groups, alkynyl groups, and aliphatic cyclic groups. Aliphatic groups may be a C₁ to C₂₀ aliphatic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). Aliphatic groups may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof. Aliphatic groups may be alkyl groups, alkenyl groups, alkynyl groups, or carbocyclic groups, and the like.

As used herein, unless otherwise indicated, the term “alkyl group” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, the alkyl group is C₁ to C₂₀, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). The alkyl group may be unsubstituted or substituted with one or more substituents. Examples of substituents include, but are not limited to, various substituents such as, for example, halogens (—F, —Cl, —Br, and —I), aliphatic groups (e.g., alkyl groups, alkenyl groups, alkynyl groups, and the like), aryl groups, alkoxide groups, carboxylate groups, carboxylic acids, ether groups, amine groups, and the like, and combinations thereof.

As used herein, unless otherwise indicated, the term “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). An aryl group may also be referred to as an aromatic group. The aryl groups may comprise polyaryl groups such as, for example, fused rings, biaryl groups, or a combination thereof. The aryl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

The present disclosure provides linear and cyclic oligoamides. The present disclosure also provides methods of making and uses of linear and cyclic oligoamides.

In an aspect, the present disclosure provides compounds. The compounds are linear or cyclic oligoamides. A compound, which may be a linear or cyclic macrocyclic oliogamide (which may also be referred to as a macrocyclic compound/oligoamide), may comprise one or more aromatic substituents. In the case where a compound comprises a plurality of aromatic substituents, adjacent aromatic substituents are linked by at least one amide group. Non-limiting examples of linear and cyclic/macrocyclic oligoamides are provided herein.

A linear oligoamide or cyclic oligoamide may a curved backbone. Not intending to be bound by any particular theory, the curved backbone is largely due to intramolecular hydrogen bonds that rigidify the amide linkage of each amide group to each aromatic substituent and at least in part to an interaction between the aromatic substituents (e.g., π-π interactions), whereby the curved backbone is stabilized.

A linear compound/oligoamide may exhibit guest-dependent folding of linear oligoamides. A guest may be a hydrogen-bond donor, such as, for example, an anion, a polar molecule, or the like. Scheme 1 shows an illustration of an example of guest-dependent folding of linear oligoamides.

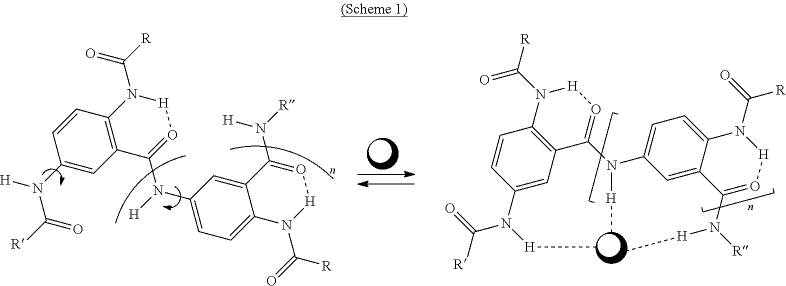

Structure on the left shows that, in the absence of a guest species capable of contributing hydrogen-bond donor(s), an oligoamide is not folded (left), i.e., the oligoamide adopts random, multiple conformations. Upon encountering a hydrogen-bond donor (e.g., anion, hydrophilic guest atom or molecule, etc.) (sphere), the backbone NH groups of the oligoamide forms hydrogen bonds with, for example, a hydrogen-bond donor (e.g., anion, hydrophilic guest atom or molecule, etc.), which “ties up” the oligoamide and forces it to adopt a crescent conformation for an oligoamide with <5 residues, or a helical conformation for an oligoamide with >5 residues. In various examples, by binding one or more hydrogen-bond donor(s) (e.g., anion(s), hydrophilic guest atom(s) or molecule(s), etc.) a linear oligoamide adopts a defined, folded conformation.

In various examples, a linear compound of the present disclosure has the following structure:

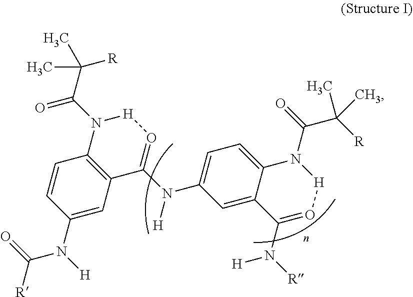

where n is 0 to 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50). R is independently at each occurrence chosen from linear aliphatic groups (which may be C₁-C₂₀ linear aliphatic groups (e.g., linear alkyl groups, such as, for example, methyl, ethyl, propyl, butyl, pentyl, hexyl, heptyl, octyl groups, and the like, which may be C₁-C₂₀ linear alkyl groups)); branched aliphatic groups (which may be C₁-C₂₀ branched aliphatic groups (e.g., branched alkyl groups, such as, for example, isopropyl, isobutyl, t-butyl, neopentyl, isopentyl groups, and the like, which may be C₁-C₂₀ branched alkyl groups)); fluorinated linear aliphatic groups (which may be C₁-C₂₀ fluorinated linear aliphatic groups (e.g., fluorinated linear alkyl groups, such as, for example, fluoromethyl, fluoroethyl, fluoropropyl, fluorobutyl, fluoropentyl, fluorohexyl, fluoroheptyl, fluorooctyl groups, and the like), which comprise one or more fluorine group(s) (e.g., a fluorinated linear aliphatic group (such as, for example, a fluorinated linear alkyl group or the like) is a perfluorinated linear aliphatic group (e.g., alkyl and the like))); fluorinated branched aliphatic groups (which may be C₁-C₂₀ fluorinated branched alkyl groups (e.g., fluorinated branched alkyl groups, such as, for example, branched derivatives of fluoropropyl, fluorobutyl, fluoropentyl, fluorohexyl, fluoroheptyl, fluorooctyl groups, and the like) and the like, which comprise one or more fluorine group(s) (e.g., a fluorinated branched aliphatic group (such as, for example, a fluorinated branched alkyl group or the like) is a perfluorinated branched aliphatic group (e.g., alkyl or the like))); ether groups (which may comprise one or two group(s) chosen from linear aliphatic groups (e.g., linear alkyl groups and the like), branched aliphatic groups (e.g., branched alkyl groups and the like), fluorinated linear aliphatic groups (e.g., fluorinated linear alkyl groups and the like), fluorinated branched aliphatic groups (e.g., fluourinated branched alkyl groups and the like) (e.g., —(CH₂)₂OCH₃, —(CH₂)₂OCH₂CH₃, —(CH₂)₂OCH₂CH(CH₃)₂, and —(CH₂)₂O(CH₂)₂CH(CH₃)₂, fluorinated analogs thereof, and the like)); and oligoether groups (which may comprise one or more group(s) chosen linear aliphatic groups (e.g., linear alkyl groups and the like), branched aliphatic groups (e.g., branched alkyl groups and the like), fluorinated linear aliphatic groups (e.g., fluorinated linear alkyl groups and the like) (e.g., ethyl groups, propyl groups, and the like and combinations thereof and/or may comprise one or more fluorine groups (e.g., the ether group may be perfluorinated)) (e.g.,

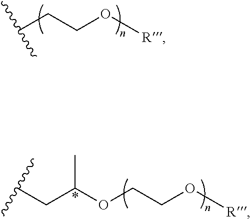

fluorinated analogs thereof, and the like, where the asterisk denotes a stereogenic carbon (i.e., a carbon having R or S stereochemistry), n is 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 8, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100), and R′″ is a linear or branched aliphatic group (e.g., alkyl group and the like) (e.g., methyl, ethyl, propyl, isopropyl, butyl, isobutyl, t-butyl, pentyl, neopentyl, isopentyl, and the like), which may comprise one or more fluorine groups (e.g., the oligoether group may be perfluorinated)), and combinations thereof.

In various examples, when the linear compound has structure I, n is 0, 1, 2, 3, or 4; R′ is chosen from methyl, ethyl, propyl, butyl, pentyl, hexyl, heptyl, octyl, and nonyl; R″ is chosen from methyl, ethyl, propyl, butyl, pentyl, and hexyl; R is —C(CH₃)₂CH₂O(CH₂)₇CH₃ or —C(CH₃)₂CH₂OR′″, where R′″ is chosen from methyl groups, ethyl groups, linear and branched propyl groups, linear and branched butyl groups, linear and branched pentyl groups, linear and branched hexyl groups, linear and branched heptyl groups, linear and branched octyl groups, and linear and branched nonyl groups.

In various examples, a compound of the present disclosure has the following structure:

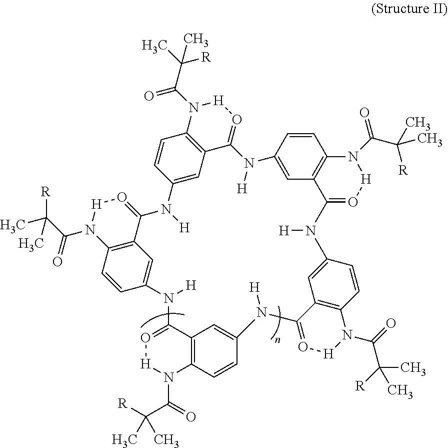

where n is 1 or 2. R is independently at each occurrence chosen from linear aliphatic groups (which may be C₁-C₂₀ linear aliphatic groups (e.g., linear alkyl groups, such as, for example, methyl, ethyl, propyl, butyl, pentyl, hexyl, heptyl, octyl groups, and the like, which may be C₁-C₂₀ linear alkyl groups)); branched aliphatic groups (which may be C₁-C₂₀ branched aliphatic groups (e.g., branched alkyl groups, such as, for example, isopropyl, isobutyl, t-butyl, neopentyl, isopentyl groups, and the like, which may be C₁-C₂₀ branched alkyl groups)); fluorinated linear aliphatic groups (which may be C₁-C₂₀ fluorinated linear aliphatic groups (e.g., fluorinated linear alkyl groups, such as, for example, fluoromethyl, fluoroethyl, fluoropropyl, fluorobutyl, fluoropentyl, fluorohexyl, fluoroheptyl, fluorooctyl groups, and the like), which comprise one or more fluorine group(s) (e.g., a fluorinated linear aliphatic group (such as, for example, a fluorinated linear alkyl group or the like) is a perfluorinated linear aliphatic group (e.g., alkyl and the like))); fluorinated branched aliphatic groups (which may be C₁-C₂₀ fluorinated branched alkyl groups (e.g., fluorinated branched alkyl groups, such as, for example, branched derivatives of fluoropropyl, fluorobutyl, fluoropentyl, fluorohexyl, fluoroheptyl, fluorooctyl groups, and the like) and the like, which comprise one or more fluorine group(s) (e.g., a fluorinated branched aliphatic group (such as, for example, a fluorinated branched alkyl group or the like) is a perfluorinated branched aliphatic group (e.g., alkyl or the like))); ether groups (which may comprise one or two group(s) chosen from linear aliphatic groups (e.g., linear alkyl groups and the like), branched aliphatic groups (e.g., branched alkyl groups and the like), fluorinated linear aliphatic groups (e.g., fluorinated linear alkyl groups and the like), fluorinated branched aliphatic groups (e.g., fluourinated branched alkyl groups and the like) (e.g., —(CH₂)₂OCH₃, —(CH₂)₂OCH₂CH₃, —(CH₂)₂OCH₂CH(CH₃)₂, and —(CH₂)₂O(CH₂)₂CH(CH₃)₂, fluorinated analogs thereof, and the like)); and oligoether groups (which may comprise one or more group(s) chosen linear aliphatic groups (e.g., linear alkyl groups and the like), branched aliphatic groups (e.g., branched alkyl groups and the like), fluorinated linear aliphatic groups (e.g., fluorinated linear alkyl groups and the like) (e.g., ethyl groups, propyl groups, and the like and combinations thereof and/or may comprise one or more fluorine groups (e.g., the ether group may be perfluorinated)) (e.g.,

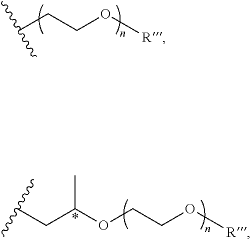

fluorinated analogs thereof, and the like, where the asterisk denotes a stereogenic carbon (i.e., a carbon having R or S stereochemistry), n is 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 8, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100), and R′″ is a linear or branched aliphatic group (e.g., alkyl group and the like) (e.g., methyl, ethyl, propyl, isopropyl, butyl, isobutyl, t-butyl, pentyl, neopentyl, isopentyl, and the like), which may comprise one or more fluorine groups (e.g., the oligoether group may be perfluorinated)), and combinations thereof.

In various examples, when a compound of the present disclosure is cyclic, n is 1. In various other examples, when a compound of the present disclosure is cyclic n is 2. In OR′ various examples, when a compound of the present disclosure is cyclic, R is

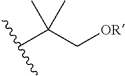

and R′ is —(CH₂)₇CH₃, —(CH₂CH₂O)₃CH₃, —CH₂CH═CH₂, methyl groups, ethyl groups, linear and branched propyl groups, linear and branched butyl groups, linear and branched pentyl groups, linear and branched hexyl groups, linear and branched heptyl groups, linear and branched octyl groups, linear and branched nonyl groups, unsaturated analogs thereof (e.g., linear and branched propenyl groups, linear and branched butenyl groups, linear and branched pentenyl groups, linear and branched hexenyl groups, linear and branched heptenyl groups, linear and branched octenyl groups, and linear and branched nonenyl groups).

A cyclic compound/oligoamide may have the same backbone as a linear compound/oligoamide of the present disclosure. Scheme 2 shows an illustration of formation of a cyclic compound/oligoamide from a linear compound/oligoamide.

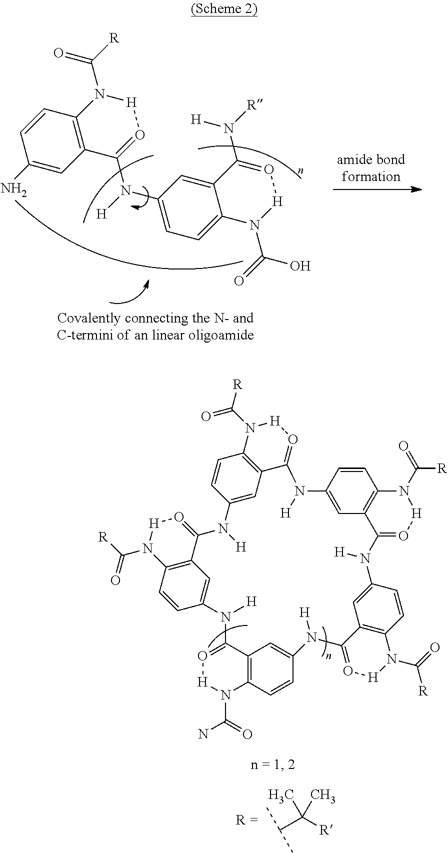

Linear oligoamide(s) may form a helical structure in the presence of one or more hydrogen-bond accepting atoms, molecules, etc. A helix can be right-handed or left-handed.

In an example, a helix comprises a compound having 5 residues (e.g., a residue is an aromatic substituent) per turn. A helix can comprise a compound having a pitch of about 3.6 Å per turn. The pitch and number of residues per turn are determined by the bond angles of the aromatic substituents. Not intending to be bound by any particular theory, these bond angles can change by several degrees, for example, depending on the temperature. As such, it is expected that the number of residues per turn and the pitch will not be exactly 5 residues and 3.6 Å, respectively, but rather the number of residues per turn and pitch will be a range surrounding these base values. For example, a helix can have 5±0.2 residues per turn, including all 0.1 residue values and ranges between 0 and 1. In another example, the helix has a pitch of 3.6±0.2 Å, including all 0.1 residue values and ranges between 0 and 1.

A helix of the present disclosure has an interior and an exterior portion. In an example, the interior of the helix is a hollow, tubular cavity comprising electropositive, hydrophilic NH groups. In an example, the exterior of the helix comprises hydrophobic groups.

The interior of the helix or the interior of the cyclic compound has a widest inner linear dimension (e.g., an inner diameter). The widest inner linear dimension of the interior is 6.4 Å (H to H), including all 0.1 Å values and ranges therebetween.

In an example, the widest inner linear dimension (e.g., an inner diameter) can vary in a compound of the present disclosure. In such an example, the helix can comprise different segments, each segment having a different widest inner linear dimension.

In an example, a helix has a longest linear dimension (e.g., a length). The longest linear dimension is 3.5 to 100 Å, including all 0.1 Å values and ranges therebetween. In another example, the longest linear dimension is 4 to 100 Å, including all 0.1 Å values and ranges therebetween.

It is desirable that one or more substituent(s) on the aromatic substituents are moderately hydrophilic. In an example, a compound of the present disclosure is soluble in a polar, aprotic solvent (e.g., N,N-dimethylformamide (DMF), dimethylsulfoxide (DMSO), and the like). For example, a compound of the present disclosure maintains solubility at a millimolar concentration (e.g., soluble at a concentration 0.1 to 10 mM, including all 0.1 mM values and ranges therebetween).

In an aspect, the present disclosure provides compositions. A composition may comprise one or more linear and/or one or more cyclic oligoamide of the present disclosure. A composition may be a pharmaceutical composition. A composition may be suitable for administration to an individual. Non-limiting examples of compositions are provided herein.

A composition may comprise one or more compound(s) or one or more complex(es) comprising one or more compound(s) and one or more hydrogen-bond acceptors and/or ions (e.g., anion(s)). In various non-limiting examples, a composition comprises one or more complexes formed by one or more compound(s) and one or more hydrogen-bond acceptors and/or ions (e.g., anion(s)). Without intending to be bound by any particular theory, it is considered that a complex is formed from (e.g., one or more interaction(s) between (e.g., one or more non-covalent interactions, such as, for example, one or more hydrogen bond(s), is formed between) the compound(s) and the hydrogen-bond acceptors and/or ions (e.g., anion(s)).

In an aspect, the present disclosure provides method of making linear and cyclic oligoamides. In various examples, a linear or cyclic amide of the present disclosure is made by a method of the present disclosure. Non-limiting examples of methods of making linear and cyclic oligoamides are provided herein.

In an aspect, the present disclosure provides uses of linear and cyclic oliogoamides. The linear and/or cyclic oliogoamides may be used in methods such as, for example, forming transmembrane pores for transmembrane transport of hydrogen-bond acceptors and/or ions, sequestering hydrogen-bond acceptors and/or ions (e.g., anions), or the like. Compounds of the present disclosure may be used to enrich materials with ions (e.g., lithium). Non-limiting examples of uses of linear and/or cyclic oligoamides are provided herein.

Linear and cyclic oligoamides may be used in environmental applications. Oligoamides are expected to bind various ions (e.g., anions). The removal of sulfate from nuclear-waste media is of both environmental and economic importance. However, due to its very high hydration energy, the sulfate ion is one of the most difficult to be extracted from aqueous media. The macrocycles, with the predisposed, multiple amide NH groups, offer a sufficiently large inner cavity that can easily accommodate the sulfate ions by forming multiple hydrogen bonds that can replace the hydration shell of this anion. It is expected that oligoamides of the present disclosure can be used in extracting sulfate ions from aqueous solution.

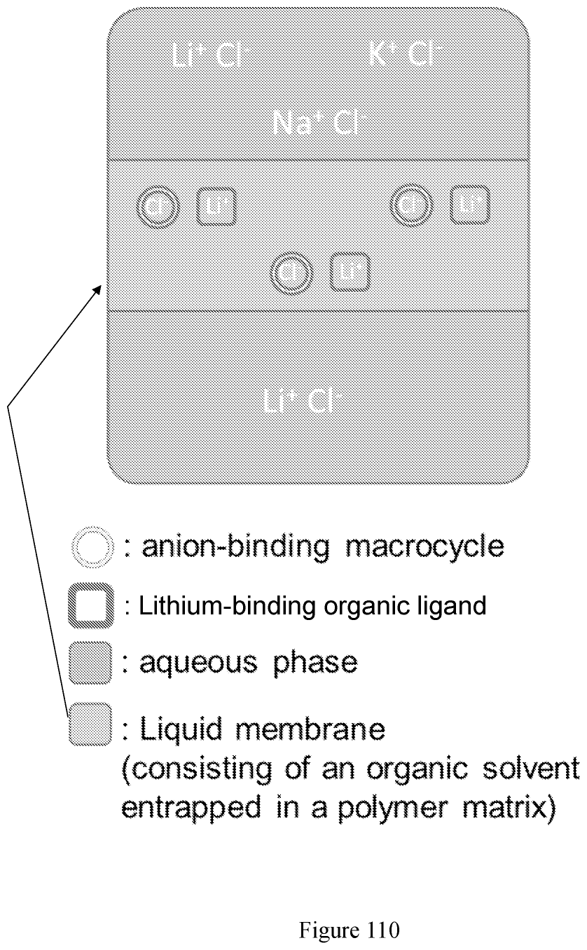

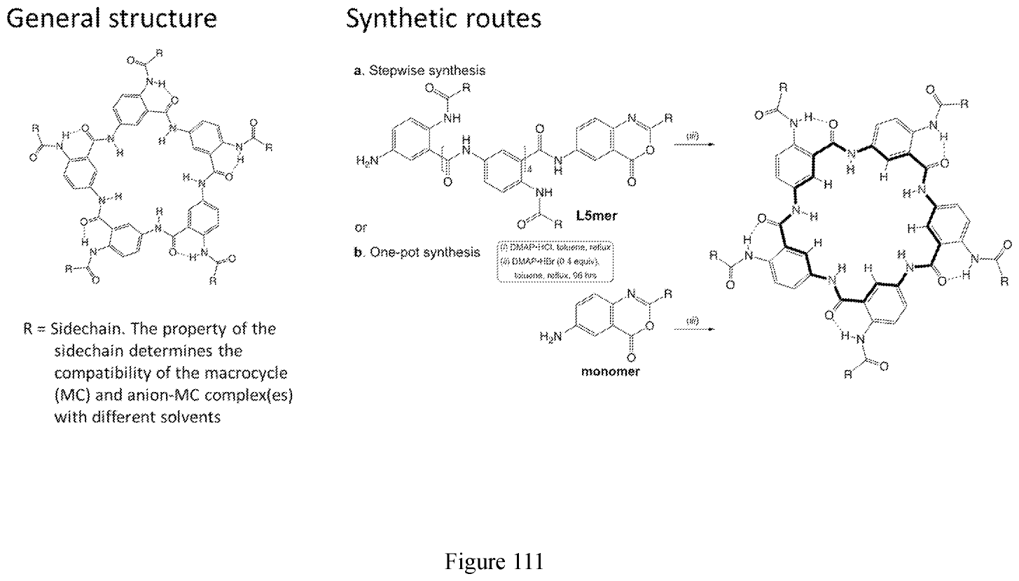

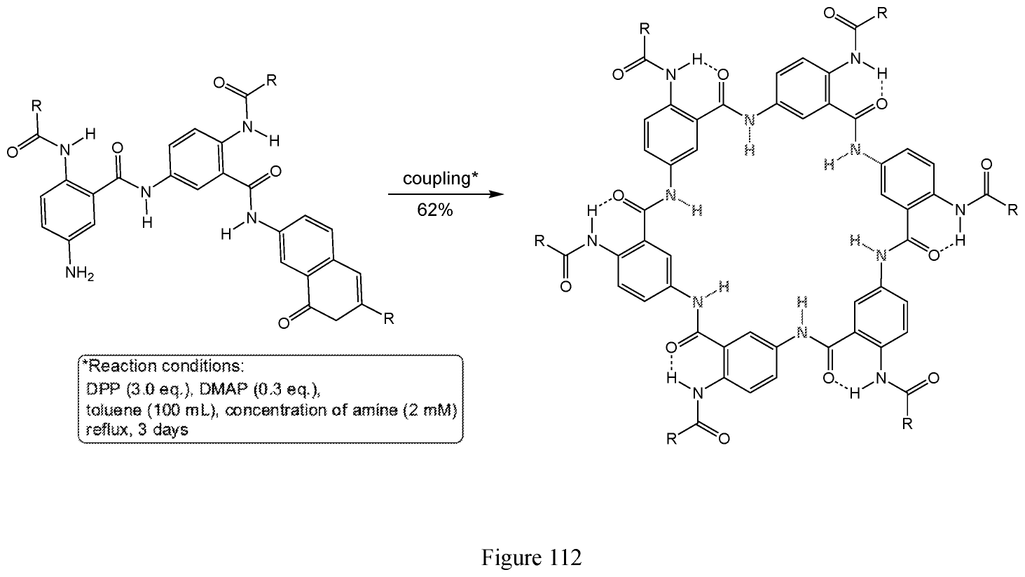

In various examples, compounds of the present disclosure are used for the enrichment of various ions, such as, for example, lithium. For example, organic binders, which are insoluble in water, are confined in the LM and serve as carriers that are not consumed. An anion binder such as the c5mer, and an organic binder specific for the lithium ion, can transfer LiCl from the first aqueous phase to the LM. A concentration gradient will drive the release of the bound LiCl in LM to the second aqueous phase as free LiCl. By selecting binders, such as crown ethers of various sizes or other reported organic cation binders, that are specific for other cations, different metal ions can also be separated or enriched. This is displayed in FIG. 110, where the “lithium-binding organic ligand” should follow the second organic binder, i.e., the “square” below the “circle” shown as the “anion-binding macrocycle.”

Compound(s) and/or composition(s) can be used to sequester various hydrogen-bond acceptors and/or ions (e.g., anions). materials. In various non-limiting examples, one or more compound(s) is/are used to sequester one or more anions(s) (such as, for example, are halide ion(s) (fluoride ion(s), chloride ion(s), bromide ion(s), iodide ion(s), or a combination thereof), nitrate ions, carbonate ions, phosphate ions, sulfate ions, oxo anions, and the like, and combinations thereof.

The hydrogen-bond acceptors and/or ions (e.g., anion(s)) may be present in an aqueous sample, in a solid sample (such as, for example, a soil sample), in a gas sample, or the like. An aqueous sample may be derived (e.g., via extraction or other methods to isolate the anion(s)) from a solid sample. An aqueous sample may be a wastewater sample (e.g., a municipal wastewater sample, industrial wastewater sample, and the like), an industrial water sample (e.g., water used to make a commercial product, such as, for example, a reagent, a solvent, or the like), a municipal water sample, or the like.

After a hydrogen-bond acceptors and/or ions binds to a compound of the present disclosure, the compound bound to the hydrogen-bond acceptors and/or ions may be referred to as a “complex.” The complexes may be removed from the aqueous sample, the solid sample, the gas sample, or the like. In various examples, the anion(s) are removed from the aqueous sample, the solid sample, the gas sample, or the like using a solid surface with one or more of the compound(s) disposed thereon.

Linear and cyclic oligoamides may be used in biological applications. In various examples, linear and cyclic oligoamides may serve as binders (receptors) that bind (sequester) hydrogen-bond acceptors and/or ions (e.g., anions) in different affinities. For example, upon wrapping around or encircling an anion, the resultant complex becomes compatible (i.e., soluble) in non-polar media and can permeate the hydrophobic interior of cell membranes, facilitating (transporting) the otherwise hydrophilic, membrane-impermeable anion to cross cell membrane. An important application of anion binders is the binding of the chloride ion. Effective binders of chloride ion can help re-balance chloride gradient across cell membranes and thus provide therapeutics for diseases such as, for example, cystic fibrosis and the like.

Linear oligoamide(s) and/or cyclic oligoamide(s) or one or more composition(s) may be used to treat an individual having an undesirable anion concentration (e.g., undesirable intra and/or extra cellular anion concentration, or the like). Compound(s) and/or compositions of the present disclosure can be administered to any human or non-human animal in need of therapy or prophylaxis for one or more condition(s) for which transport of one or more anion(s) is intended to provide a prophylactic or therapeutic benefit. Thus, the individual can be diagnosed with, suspected of having, or be at risk for developing any of a variety of conditions for which a reduction in severity would be desirable. Non-limiting examples of such conditions include cystic fibrosis, and other diseases known to be associated with defects in anion channels. Additional examples of such diseases may be found in C. A. Hubner, and T. J. Jentsch, Human Molecular Genetics, 2002, Vol. 11, 2435-2445), the relevant portions thereof are incorporated herein by reference.

In an example, one or more composition(s) of the present disclosure are delivered (e.g., administered) to an individual. Methods of administration are known in the art and non-limiting examples of which are described herein.

As described herein, compounds and/or compositions of the present disclosure can be provided in pharmaceutical compositions for administration by combining them with any suitable pharmaceutically acceptable carriers, excipients, stabilizers, or a combination thereof. Examples of pharmaceutically acceptable carriers, excipients, and stabilizers can be found in Remington: The Science and Practice of Pharmacy (2005) 21st Edition, Philadelphia, PA. Lippincott Williams & Wilkins. For example, suitable carriers include excipients and stabilizers which are nontoxic to recipients at the dosages and concentrations employed, and include buffers such as, for example, acetate, Tris, phosphate, citrate, and other organic acids; antioxidants including ascorbic acid and methionine; preservatives such as, for example, octadecyldimethylbenzyl ammonium chloride; hexamethonium chloride; benzalkonium chloride, benzethonium chloride; phenol, butyl or benzyl alcohol; alkyl parabens such as, for example, methyl or propyl paraben; catechol; resorcinol; cyclohexanol; 3-pentanol; and m-cresol; amino acids such as, for example, glycine, glutamine, asparagine, histidine, arginine, or lysine; monosaccharides, disaccharides, and other carbohydrates including glucose, mannose, or dextrins; chelating agents such as, for example, EDTA; tonicifiers such as, for example, trehalose and sodium chloride; sugars such as, for example, sucrose, mannitol, trehalose or sorbitol; surfactant such as, for example, polysorbate; salt-forming counter-ions such as, for example, sodium; and/or non-ionic surfactants such as, for example, Tween or polyethylene glycol (PEG). Additional examples of pharmaceutically acceptable carriers include, but are not limited to, sugars, such as lactose, glucose, and sucrose; starches, such as corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The pharmaceutical compositions may comprise other therapeutic agents. The present compositions can be provided as single doses or in multiple doses covering the entire or partial treatment regimen. The compositions can be provided in liquid, solid, semi-solid, gel, aerosolized, vaporized, or any other form from which it can be delivered to an individual.

The compositions may be administered parenterally. Administration of formulations comprising compounds and/or compositions as described herein can be carried out using any suitable route of administration known in the art. For example, the formulations comprising compounds and/or compositions of the present disclosure are administered via intravenous, intramuscular, intraperitoneal, intracerobrospinal, subcutaneous, intra-articular, or intrasynovial, routes. The compositions may be introduced as a single administration or as multiple administrations or may be introduced in a continuous manner over a period of time. For example, the administration(s) can be a pre-specified number of administrations or daily, weekly or monthly administrations, which may be continuous or intermittent, as may be clinically needed and/or therapeutically indicated.

Methods of the present disclosure may be used on various individuals. In various examples, an individual is a human or non-human mammal. Examples of non-human mammals include, but are not limited to, farm animals, such as, for example, cows, hogs, sheep, and the like, as well as pet or sport animals such as, for example, horses, dogs, cats, and the like. Additional non-limiting examples of individuals include, but are not limited to, rabbits, rats, mice, and the like.

The steps of the methods described in the various embodiments and examples disclosed herein are sufficient to produce a polymer of the present disclosure or carry out a method of the present disclosure. Thus, in various embodiments, a method consists essentially of a combination of the steps of the methods disclosed herein. In various other embodiments, a method consists of such steps.

The following Statements describe various examples and embodiments of the present disclosure.

Statement 1. A compound comprising one or more aromatic substituents, wherein in the case of a plurality of aromatic substituents, adjacent aromatic substituents are linked by at least one amide group, wherein the compound has the structure:
(which may be referred to a linear or helical compound or a linear or helical oligoamide)

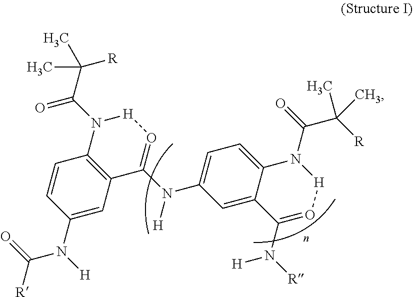

wherein n is 0 to 50 (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) or
(which may be referred to as a cyclic compound or a cyclic oligoamide)

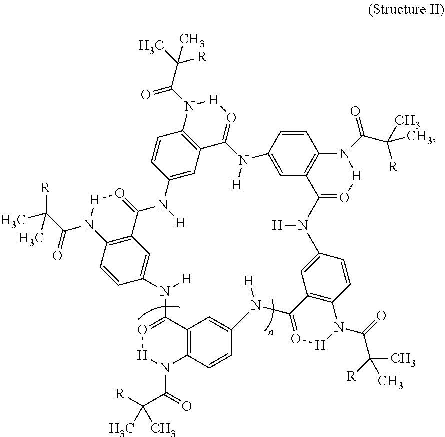

wherein n is 1 or 2, and
wherein R is independently at each occurrence chosen from: linear aliphatic groups, which may be C₁-C₂₀ linear aliphatic groups (e.g., linear alkyl groups, such as, for example, methyl, ethyl, propyl, butyl, pentyl, hexyl, heptyl, octyl groups, and the like, which may be C₁-C₂₀ linear alkyl groups); branched aliphatic groups, which may be C₁-C₂₀ branched aliphatic groups (e.g., branched alkyl groups, such as, for example, isopropyl, isobutyl, t-butyl, neopentyl, isopentyl groups, and the like, which may be C₁-C₂₀ branched alkyl groups); fluorinated linear aliphatic groups, which may be C₁-C₂₀ fluorinated linear aliphatic groups (e.g., fluorinated linear alkyl groups, such as, for example, fluoromethyl, fluoroethyl, fluoropropyl, fluorobutyl, fluoropentyl, fluorohexyl, fluoroheptyl, fluorooctyl groups, and the like, and the like), which comprise one or more fluorine group(s) (e.g., a fluorinated linear aliphatic group (such as, for example, a fluorinated linear alkyl group or the like) is a perfluorinated linear aliphatic (e.g., alkyl and the like) group); fluorinated branched aliphatic groups, which may be C₁-C₂₀ fluorinated branched alkyl groups (e.g., fluorinated branched alkyl groups, such as, for example, branched derivatives of fluoropropyl, fluorobutyl, fluoropentyl, fluorohexyl, fluoroheptyl, fluorooctyl groups, and the like) and the like, which comprise one or more fluorine group(s) (e.g., a fluorinated branched aliphatic group (such as, for example, a fluorinated branched alkyl group or the like) is a perfluorinated branched aliphatic (e.g., alkyl or the like) group); ether groups (which may comprise one or two group(s) chosen from linear aliphatic groups (e.g., linear alkyl groups and the like), branched aliphatic groups (e.g., branched alkyl groups and the like), fluorinated linear aliphatic groups (e.g., fluorinated linear alkyl groups and the like), fluorinated branched aliphatic groups (e.g., fluourinated branched alkyl groups and the like)
(e.g., —(CH₂)₂OCH₃, —(CH₂)₂OCH₂CH₃, —(CH₂)₂OCH₂CH(CH₃)₂, and —(CH₂)₂O(CH₂)₂CH(CH₃)₂, fluorinated analogs thereof, and the like); and oligoether groups (which may comprise one or more group(s) chosen linear aliphatic groups (e.g., linear alkyl groups and the like), branched aliphatic groups (e.g., branched alkyl groups and the like), fluorinated linear aliphatic groups (e.g., fluorinated linear alkyl groups and the like) (e.g., ethyl groups, propyl groups, and the like and combinations thereof and/or may comprise one or more fluorine groups (e.g., the ether group may be perfluorinated)) (e.g.,

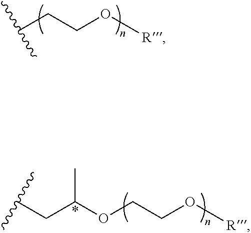

fluorinated analogs thereof, and the like, wherein the asterisk denotes a stereogenic carbon (i.e., a carbon having R or S stereochemistry), n is 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 8, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100), and R′″ is a linear or branched aliphatic group (e.g., alkyl group and the like) (e.g., methyl, ethyl, propyl, isopropyl, butyl, isobutyl, t-butyl, pentyl, neopentyl, isopentyl, and the like), which may comprise one or more fluorine groups (e.g., the oligoether group may be perfluorinated), and combinations thereof, and wherein R′ and R″ are independently chosen from linear or branched aliphatic groups, aryl group (e.g., phenyl groups, substituted aryl groups, such as, for example, o-, m- or p-tolyl groups, o-, m-, or p-halo groups, methoxyphenyl groups, and the like)). Upon binding an anion, the compound may have a curved or helical backbone also due at least in part to intramolecular hydrogen bonds (e.g., between the sidechain amide NH groups and the backbone amide carbonyl groups) that contribute to rigidify the amide linkage of each amide group to each aromatic substituent and at least in part to an interaction between the aromatic substituents, whereby the curved backbone is stabilized along with intermolecular hydrogen bonds between the backbone NH groups and the anion. In various examples, when the compound has Structure I, n is 0, 1, 2, 3, or 4; the R′ group is a linear or branched methyl group, linear or branched ethyl group, linear or branched propyl groups, linear or branched butyl groups, linear or branched pentyl groups, linear or branched hexyl groups, linear or branched heptyl groups, linear or branched octyl groups, or linear or branched nonyl groups; the R″ group is a methyl group, ethyl group, linear or branched propyl groups, linear or branched butyl groups, linear or branched pentyl groups, or linear or branched hexyl groups; and the R group is —C(CH₃)₂CH₂O(CH₂)₇CH₃ or —C(CH₃)₂CH₂OR′″, where R′″ is chosen from methyl groups, ethyl groups, linear and branched propyl groups, linear and branched butyl groups, linear and branched pentyl groups, linear and branched hexyl groups, linear and branched heptyl groups, linear and branched octyl groups, and linear and branched nonyl groups. In various examples, when the compound has structure II, n is 1, R is

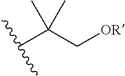

and R′ is —(CH₂)₇CH₃, —(CH₂CH₂O)₃CH₃, —CH₂CH═CH₂, methyl groups, ethyl groups, linear and branched propyl groups, linear and branched butyl groups, linear and branched pentyl groups, linear and branched hexyl groups, linear and branched heptyl groups, linear and branched octyl groups, linear and branched nonyl groups, unsaturated analogs thereof (e.g., linear and branched propenyl groups, linear and branched butenyl groups, linear and branched pentenyl groups, linear and branched hexenyl groups, linear and branched heptenyl groups, linear and branched octenyl groups, and linear and branched nonenyl groups). In various examples, when the compound has structure II, n is 2, R is

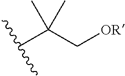

and R′ is —(CH₂)₇CH₃, —(CH₂CH₂O)₃CH₃, —CH₂CH═CH₂, methyl groups, ethyl groups, linear and branched propyl groups, linear and branched butyl groups, linear and branched pentyl groups, linear and branched hexyl groups, linear and branched heptyl groups, linear and branched octyl groups, linear and branched nonyl groups, unsaturated analogs thereof (e.g., linear and branched propenyl groups, linear and branched butenyl groups, linear and branched pentenyl groups, linear and branched hexenyl groups, linear and branched heptenyl groups, linear and branched octenyl groups, and linear and branched nonenyl groups).
Statement 2. A composition comprising one or more compound(s) of the present disclosure (e.g., one or more compound(s) of according to Statement 1) and one or more hydrogen-bond acceptor(s) (e.g., polar guest molecule(s), anion(s), or the like), wherein the backbone of the compound has a crescent conformation or helical conformation (e.g., form a left-handed or right-handed helix) (e.g., extending longitudinally in the direction of a longitudinal axis). In the absence of an anion or a polar molecule providing hydrogen bond acceptors, a compound (i.e., a non-cyclic or linear oligoamide) adopts random (i.e., multiple) conformations. The backbone of the compound (a linear oligoamide) folds in the presence of an anion or a polar molecule offering hydrogen-bond acceptor(s) that form(s) one or more hydrogen bond(s) with the backbone NH groups of the compound.
Statement 3. A composition according to Statement 2, wherein the hydrogen-bond acceptor(s) and one or more anion(s) (such as, for example, halide ion(s) (fluoride ion(s), chloride ion(s), bromide ion(s), iodide ion(s), or a combination thereof), nitrate ions, carbonate ions, phosphate ions, sulfate ions, oxo anions, and the like, and combinations thereof).
Statement 4. A composition according to Statements 2 or 3, wherein the compound has a linear structure that adopts a crescent or helical conformation has an interior and an exterior of the crescent or helix, and intramolecular hydrogen bonds are on the exterior of the helix and intermolecular hydrogen bonds are on the interior of the crescent or helix.
Statement 5. A composition according to any one of Statements 2-4, wherein the helix has about −5 residues per turn and/or a pitch of about 3.6 Å per turn.
Statement 6. A composition according to any one of Statements 2-5, wherein the interior of a helical conformation defines a hollow tubular cavity that is parallel to the longitudinal axis.
Statement 7. A composition according to any one of Statements 2-6, wherein the interior has an inner diameter of ˜6.5 Å, including all 0.1 Å value and range therebetween.
Statement 8. A composition according to any one of Statements 2-7, wherein the compound has a length (e.g., a length along the longitudinal axis) of 3.5 to 100 Å, including all 0.1 Å value and range therebetween.
Statement 9. A composition according to any one Statements 2-8, wherein the interior (also called the inner pore) is electrostatically positive and hydrophilic, and the exterior is hydrophobic.
Statement 10. A composition according to any one of the preceding Statements, wherein the composition comprises a plurality of compounds that all have the same structure (e.g., share the same type of oligoamide backbone), at least one of the compounds has a different structure, or all of the compounds are different.
Statement 11. A method of sequestering one or more anion(s) comprising: contacting one or more anion(s) with one or more compound(s) of the present disclosure (e.g., one or more compounds of Statement 1), wherein at least a portion or all of the anion(s) are sequestered by the compound(s). The anion(s) may independently by bound by a compound. The compound(s) may be disposed on a substrate (e.g., conjugated to a substrate).
Statement 12. A method of sequestering one or more anion(s) according to Statement 11, wherein during the contacting the anion(s) are present in a sample.
Statement 13. A method of sequestering one or more anion(s) according to Statements 11 or 12, wherein the sample is an organic or aqueous solution. In various examples, the anion(s) are halide ion(s) (fluoride ion(s), chloride ion(s), bromide ion(s), iodide ion(s), or a combination thereof), nitrate ions, carbonate ions, phosphate ions, sulfate ions, oxo anions, and the like, and combinations thereof.
Statement 14. A method of sequestering one or more anion(s) according to any one of Statements 11-13, wherein the sample is a solution such as a wastewater sample (e.g., a municipal wastewater sample, industrial wastewater sample, and the like), an industrial water sample (e.g., water used to make a commercial product, such as, for example, a reagent, a solvent, or the like), a municipal water sample, a solution in organic solvent, or the like.
Statement 15. A method of sequestering one or more anion(s) according to any one of Statement 11-14, wherein a complex is formed from (e.g., one or more interaction(s) between (e.g., one or more non-covalent bond(s) is/are formed between) the compound(s) and anion(s).
Statement 16. A method of sequestering one or more anion(s) according to any one of Statements 11-15, herein the sequestered anions(s) (e.g., complex) is/are isolated (e.g., removed from the sample or the like).
Statement 17. A method of treating an individual diagnosed with or suspected of having an extracellular and/or intracellular anion imbalance comprising: administering to the individual one or more compound(s) of the present disclosure (e.g., one or more compound(s) according to Statement 1) to the individual. In an example, the individual is in need of treatment for an indication where the intracellular and/or extracellular concentration of an anion (typically, chloride ion) is unbalanced (e.g., physiologically undesirable) (e.g., the individual has been diagnosed with cystic fibrosis or the like). Without intending to be bound by any particular theory, it is considered that compound(s) of the present disclosure binds anions and facilitate transport of the bound anions across cell membranes, which results in correction of imbalanced extracellular and/or intracellular anion concentration gradients (e.g., the extracellular and/or intracellular anion imbalance is adjusted).
Statement 18. The method of Statement 17, wherein the physiological gradient of anion concentration in the individual is at least partially or completely restored.
Statement 19. The method of Statements 17 or 18, wherein one or more symptom(s) related to the extracellular and/or intracellular anion imbalance in the individual is at least partially or completely alleviated.
Statement 20. A method of sequestering one or more anion(s) according to any one of Statements 17-19, wherein the individual is a human or a non-human animal (e.g., a human mammal, a non-human mammal, or the like).
Statement 21. A method of sequestering one or more anion(s) according to any one of Statements 17-20, herein the sequestered anions(s) (e.g., complex) is/are isolated (e.g., removed from the sample or the like).
Statement 22. A method of sequestering one or more hydrogen-bond acceptors and/or ions comprising: contacting the one or more hydrogen-bond acceptors and/or ions with one or more compound(s) according to Statement 1, wherein at least a portion or all of the one or more hydrogen-bond acceptors and/or ions are sequestered by the compound(s).
Statement 23. A method according to Statement 22, wherein the compound(s) are disposed on a substrate.
Statement 24. A method according to Statements 22 or 23, wherein a sample comprises the one or more hydrogen-bond acceptors and/or ions and the sample is an organic or aqueous solution.
Statement 25. A method according to Statement 24, wherein the sample is a wastewater sample, an industrial water sample, a municipal water sample, or a solution in organic solvent.
Statement 26. A method according to any one of Statements 22-25, wherein a complex is formed from the compound(s) and one or more hydrogen-bond acceptors and/or ions.
Statement 27. A method according to any one of Statements 22-26, wherein the sequestered one or more hydrogen-bond acceptors and/or ions is/are isolated.
Statement 28. A method of treating an individual diagnosed with or suspected of having an extracellular and/or intracellular anion imbalance comprising: administering to the individual one or more compound(s) according to Statement 1 to the individual.
Statement 29. A method according to Statement 28, wherein the individual has been diagnosed with cystic fibrosis.
Statement 30. A method according to Statements 28 or 29, wherein the physiological gradient of anion concentration in the individual is at least partially or completely restored.
Statement 31. A method according to any one of Statements 28-30, wherein one or more symptom(s) related to the extracellular and/or intracellular anion imbalance in the individual is at least partially or completely alleviated.
Statement 32. A method according to any one of Statements 28-31, wherein the individual is a human or a non-human animal.
Statement 33. A method according to any one of Statements 28-32, wherein the sequestered anions(s) is/are isolated.

The following examples are presented to illustrate the present disclosure. The examples are not intended to be limiting in any matter.

### Example 1

This example provides a description of compounds of the present disclosure.

Described herein is the design, synthesis, and study of a hitherto unknown series of aromatic oligoamides that fold upon binding anions. As shown in FIG. 1a, inverting the orientation of the backbone amide groups of A leads to oligoamide H in which a six-membered intramolecular H-bond is introduced between each backbone amide oxygen and the amide proton of the adjacent acylamino sidechain. Such an intramolecular H-bond keeps the backbone amide oxygen atom from engaging in additional H-bonding and, more importantly, frees each backbone NH group to engage in H-bonding with other guest species.

Unlike that of A, the backbone of oligoamide H is partially constrained. Around each backbone amide group of H, the rotation of the aryl-CO single bond is limited by an intramolecular H-bond, while the rotation of the C(O)NH-aryl bond remains unhindered. It is thus expected that H will adopt multiple conformations which is entropically favorable (FIG. 1b). Upon adding a guest, such as an anion that forms strong H-bonds with the backbone amide protons, the entropic barrier for adopting a single conformation is overcome by the enthalpic contribution from the multiple H-bonds between the guest and the oligoamide host. As a result, the equilibrium is shifted toward complex H·G, resulting in a folded conformation for H.

In contrast to A and other aromatic oligoamide foldamers, which can be synthesized based on established amide chemistry, oligoamides H, which comprise 5-amino-N-acylanthranilic acid residues, cannot be obtained by using known methods. It was reported that, when being treated with acylating or coupling reagents, anthranilic acid and its N-acylated derivatives self-cyclize into derivatives having a (4H)-3, 1-benzoxazin-4-one (or benzoxazinone) core, which prevents amide coupling from happening.

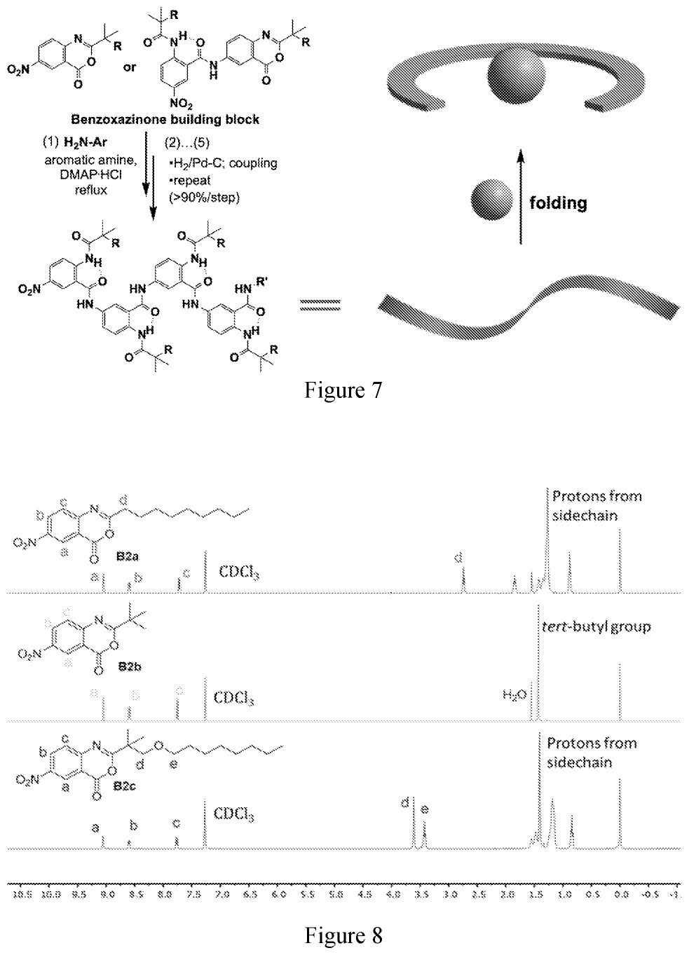

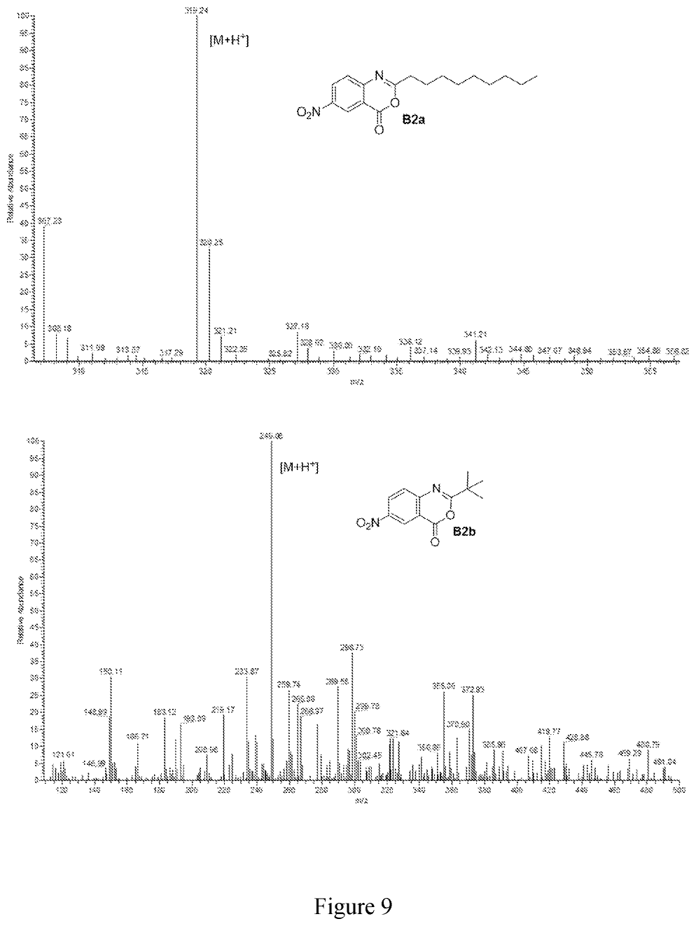

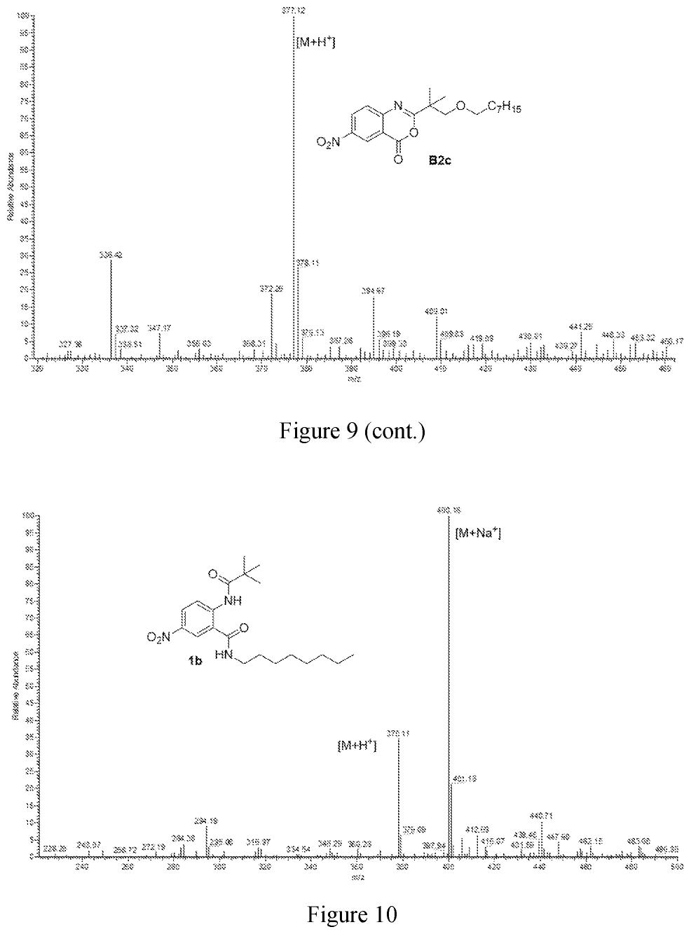

Indeed, treating 5-nitro-anthranilic acid B1 with two or more equivalents of decanoyl or trimethylacetyl chloride gave benzoxazinone derivatives B2a or B2b (Scheme 1). Similarly, compound B2c was obtained by converting B1 into the corresponding 5-nitro-N-acylanthranilic acid which was then treated with acetyl chloride. The ¹H NMR spectra of B2a-c reveal the same benzoxazinone core (FIG. 8). The identities of these compounds are also verified with ESI-MS spectra (FIG. 9).

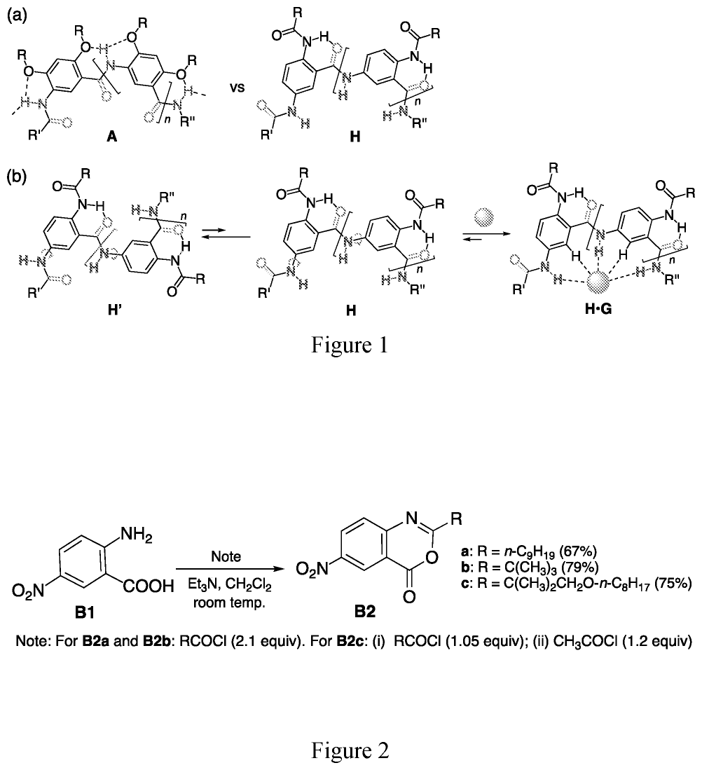

Single crystals of B2b were obtained from hexane/ethyl acetate (1/2, v/v) by slow evaporation of solvents at room temperature. The X-ray structure of B2b confirms that acid B1 was converted into the benzoxazinone derivative by treating with trimethylacetyl chloride (FIG. 2a).

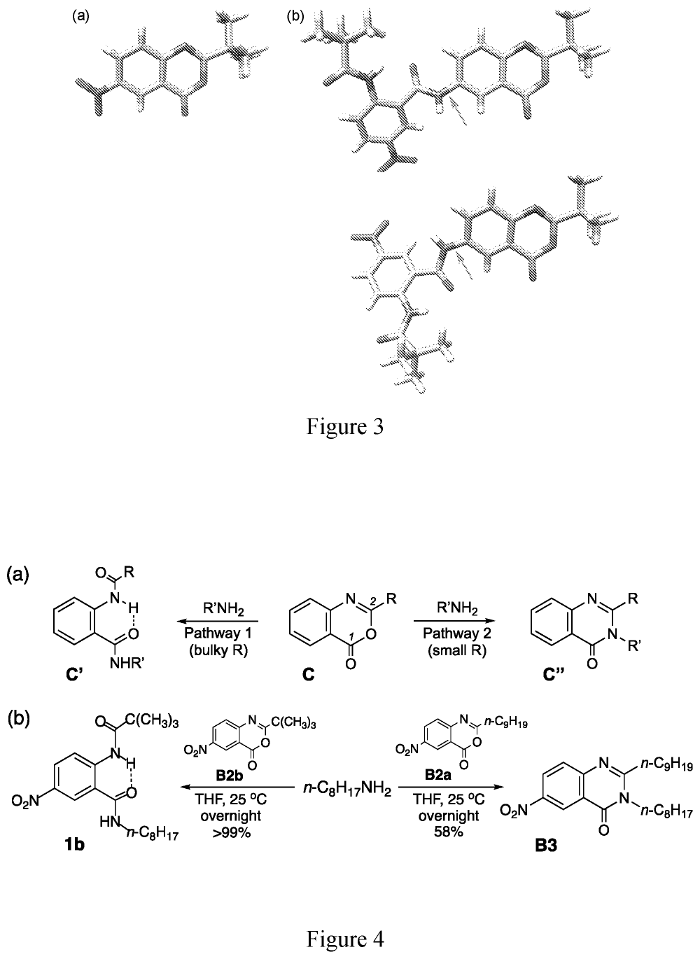

While anthranilic acid or its N-acyl derivatives cannot directly couple with other amines due to self-cyclization, benzoxazinone C and its derivatives do react with amines via two routes (FIG. 3a). One involves nucleophilic attack at carbon C-1, leading to ring-opening product C′; the other involves attacking C-2 to give quinazolinone C″. Steric factors determine the outcome of a reaction. With bulky R and/or R′, the desired ring-opening of C happens, forming product C′ having an acylamino (RCONH—) sidechain and a new amide bond; with small or slim R and/or R′ that do not impose significant steric hindrance, carbon C-2 is attacked to give C″ with no amide bond.

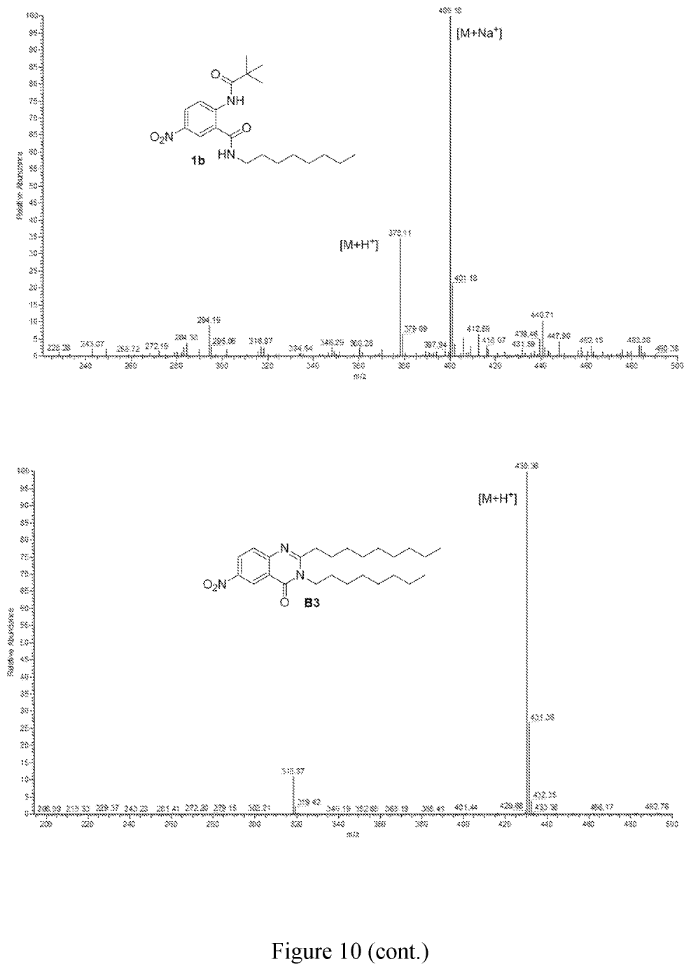

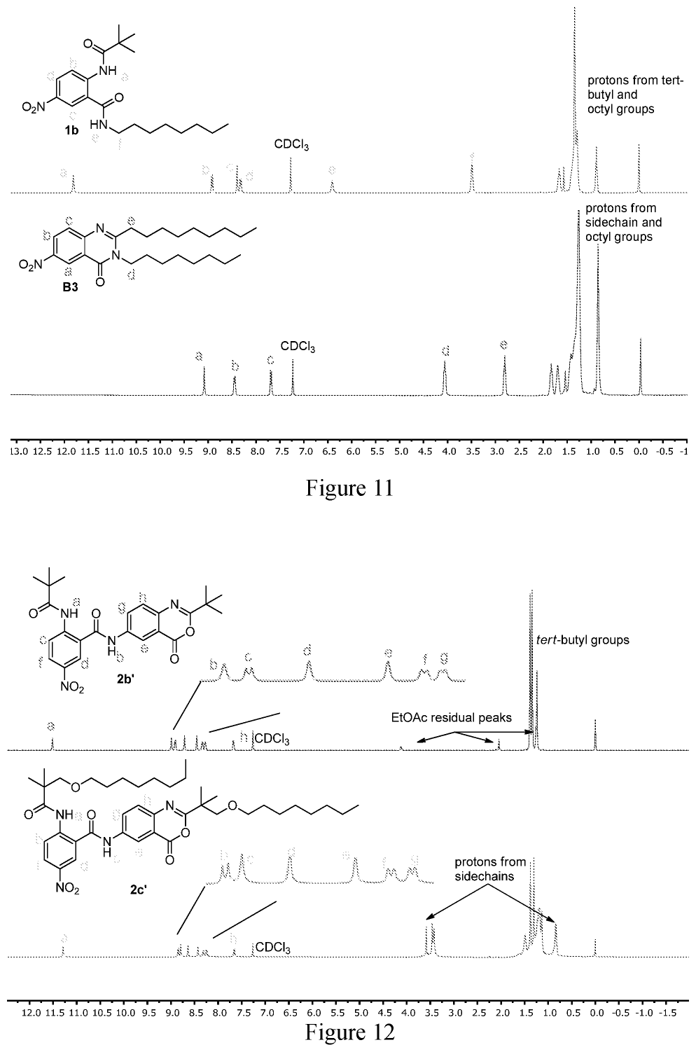

Compound B2a and B2b carrying an n-octyl and a t-butyl side chains, respectively, were treated with n-octylamine (FIG. 3b). The reaction of B2a and n-octylamine afforded quinazolinone B3 in 58% yield, while the reaction of B2b and n-octylamine led to the ring-opening product 1b nearly quantitatively. The molecular weights of B3 and 1b are confirmed by mass spectra (FIG. 10). ¹H NMR spectra (FIG. 11) reveal two amide signals at 6.33 and 11.79 ppm for 1b, with no amide signal observed with B3. Thus, to ensure the formation of the amide bond and the release of the acylamino side chain, as observed with amide 1b, a bulky R group, like that of B2b, with a tertiary a-carbon should be present.

Based on the observation made with B2b, the synthesis of oligoamides having the backbone of general structure H was probed (FIG. 5a). Treating B2b and the amine derived from 1b in the presence of 4-dimethylaminopyridine hydrochloride (DMAP·HCl) gave dimer 2b in 98% yield. Reducing 2b to its corresponding amine followed by coupling with B2b gave trimer 3b in 95% yield. However, coupling B2b to the amine from trimer 3b led to a poorly soluble product, presumably the corresponding tetramer, that defied characterization. Dimer 2c to pentamer 5c carrying sidechains having a quaternary u-carbon and an n-octyloxy tail were obtained >90% yields by stepwise coupling of B2c to the amine precursors under the same conditions. Oligoamides 2c to 5c showed good solubility in solvents including chloroform and DMSO.

Dimeric 2b′ and 2c′, each having a benzoxazinone moiety, were also prepared (below) and characterized (FIG. 12). The solid-sate structure of 2b′ confirms the presence of the benzoxazinone moiety (FIG. 2b), in which two conformations related by ˜180° rotation around the single bond between the benzoxazinone unit and the rest of the molecule, are revealed.

Under the same conditions for coupling B2b or B2c, treating 2c′ and tetramer amine 4c-NH₂ gave hexamer 6c in 92% yield (FIG. 5b). The synthesis of 2b, 3b, and 2c-6c indicates that the adopted strategy, which involves repetitive coupling of benzoxazinone monomers B2b and B2c to a growing oligomer chain, or the coupling of oligomers 2c′ and 4c-NH₂, represents a new, highly efficient method for forming amide bonds. This method, which involves refluxing without any coupling reagent, is straightforward and convenient for synthesizing these new aromatic oligoamides.

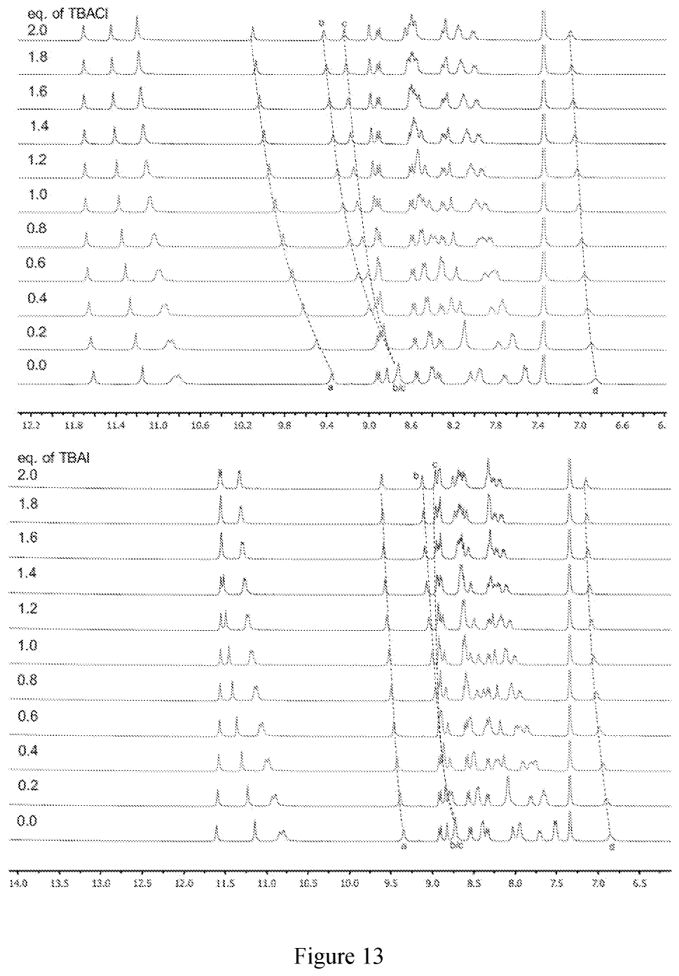

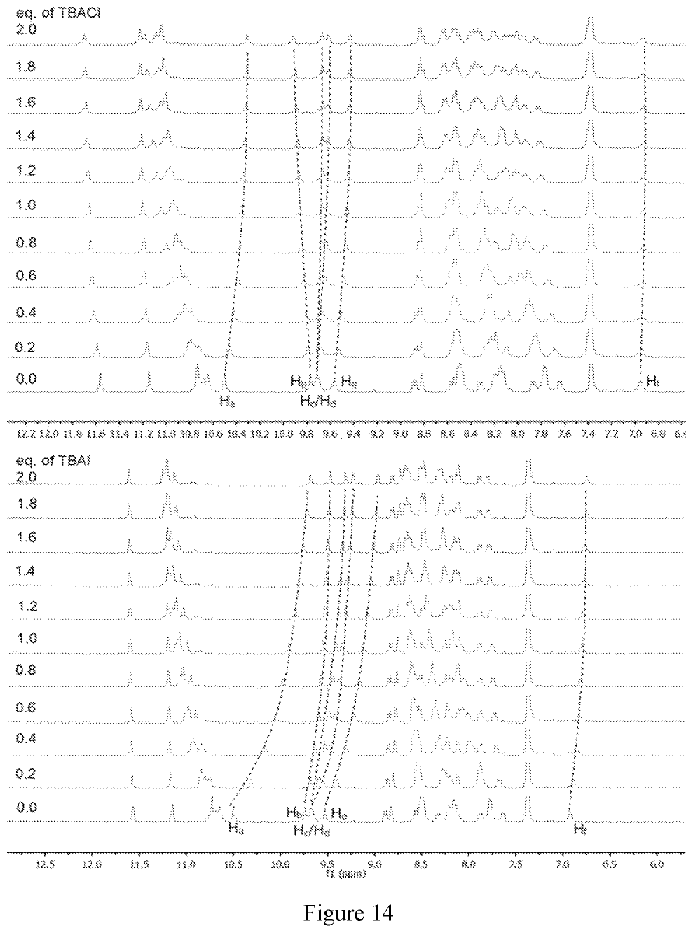

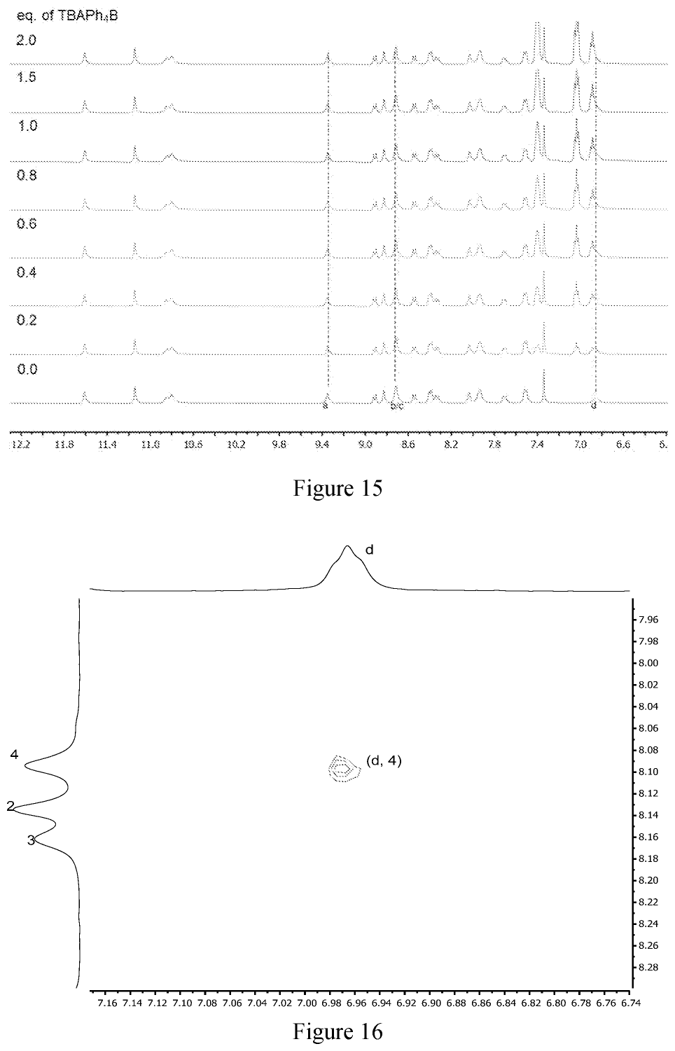

The interaction of the obtained oligoamides with anions was explored by titrating 4c (5 mM) with tetra-n-butylamnonium (n-Bu₄N⁺) chloride or iodide. In CD₃CN/CDCl₃ (1/9, v/v), the signals of the backbone amide protons of 4c exhibit downfield shifts with 0 to 2.0 equivalents of n-Bu₄N⁺Cl⁻ (FIG. 13) or n-Bu₄N⁺I⁻ (FIG. 14), indicating that the chloride or iodide ions engage in H-bonding interactions with these amide protons. In contrast, titrating 4c (5 mM) with 0 to 2 equivalents of tetra-n-butylammonium tetraphenyl borate (n-Bu₄N⁺Ph₄B⁻), which cannot form H-bond, did not lead to any shift in the amide proton resonances (FIG. 15). These results demonstrate that 4c indeed bind anions like chloride and iodide.

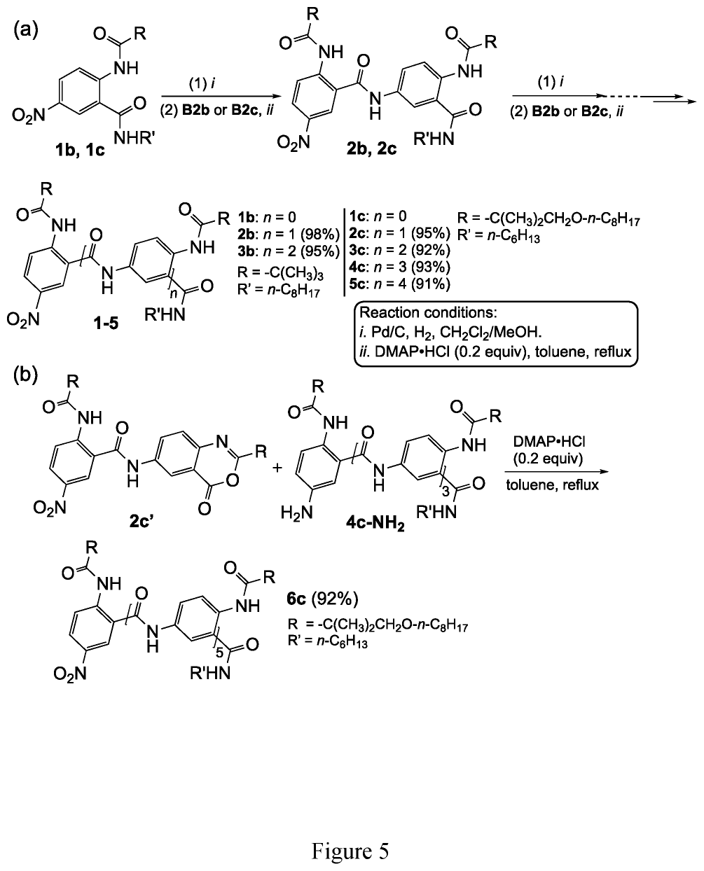

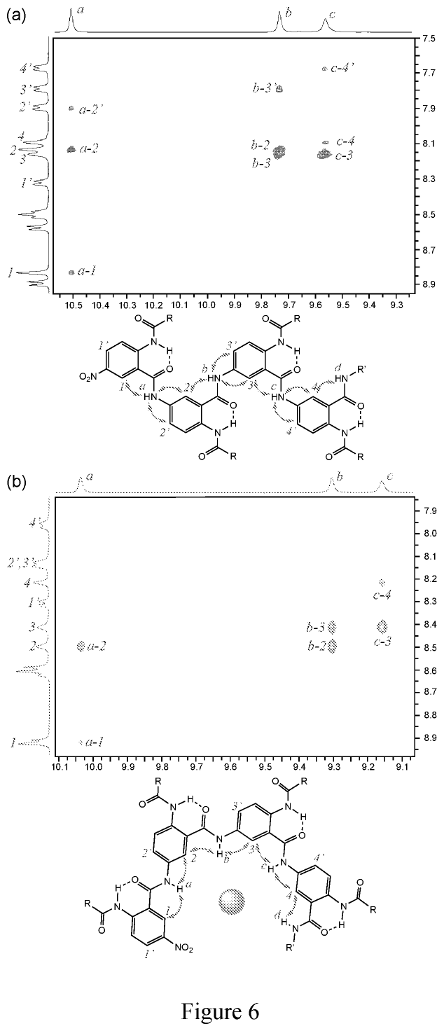

The conformational change of tetramer 4c upon binding anions was probed by comparing the 2D (NOESY) spectrum of 4c in the absence and presence of one equivalent of n-Bu₄N⁺I⁻. In CDCl₃ containing 5% DMSO-d₆, the NOESY spectrum of free 4c reveals three NOEs involving each of amide protons a, b, and c and its neighboring aromatic protons (FIG. 4a). Among the NOEs, those involving protons a and 1, b and 2, and c and 3, along with that between protons d and 4 (FIG. 16), are expected because of the presence of the intramolecular H-bond between each backbone amide carbonyl group and the adjacent NH group of the sidechain. In addition, two NOEs involving amide proton a, b, or c, and the two “outer” (protons 1′-3′) and “inner” (protons 1-3) aromatic protons are observed, consistent with the expected rotation of each (CO)NH-aryl single bond in 4c (FIG. 4a). These observed NOEs indicate that, due to the rotation of the (CO)NH-aryl bonds, oligoamide 4c most likely adopes random conformations roughly represented by the zig-zagged structure in FIG. 5a.

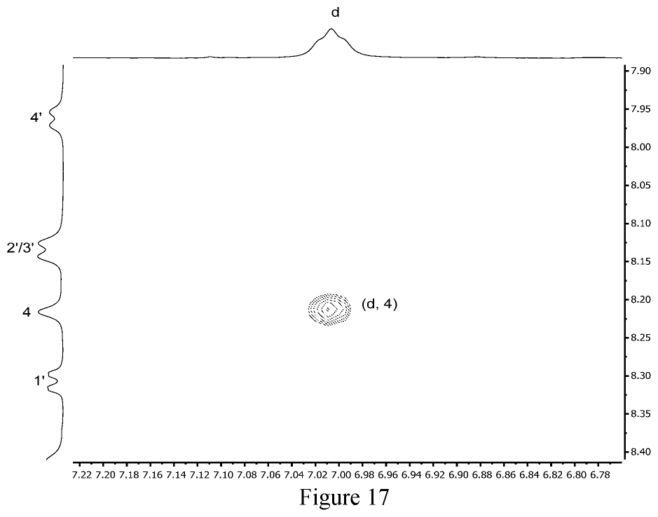

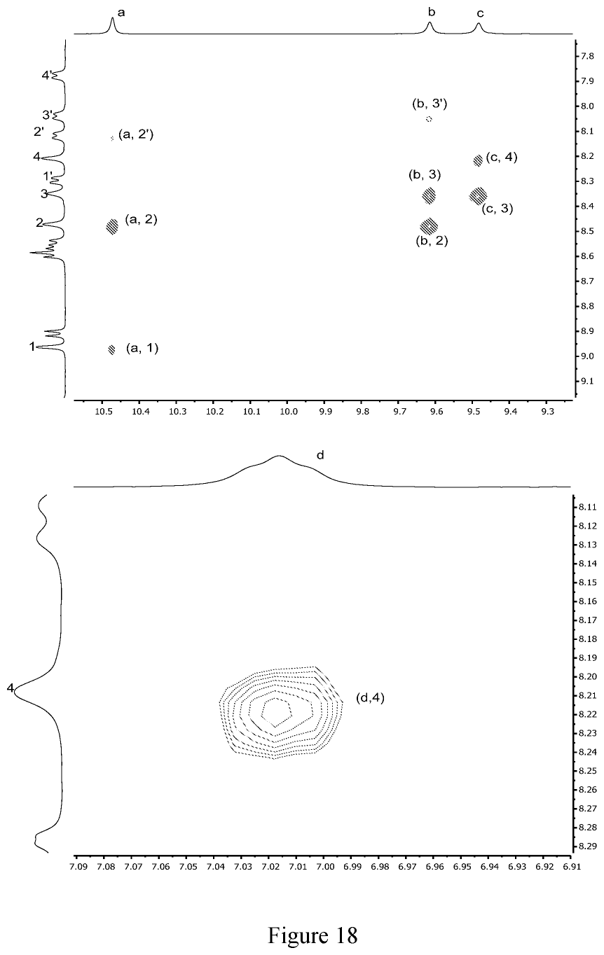

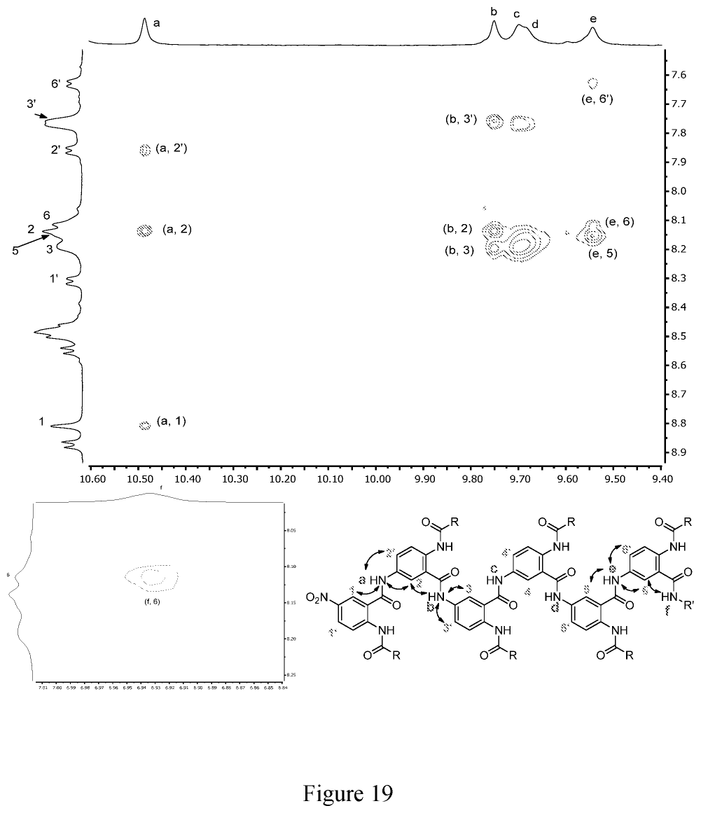

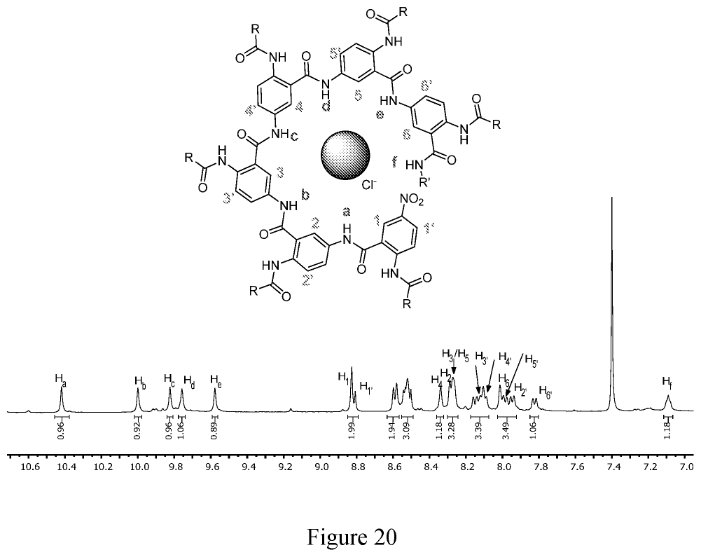

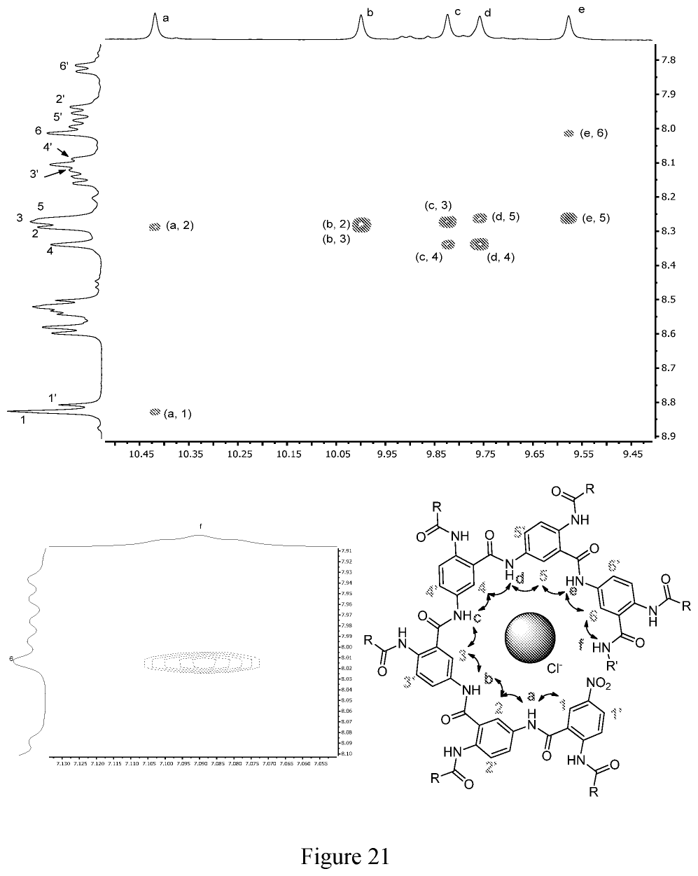

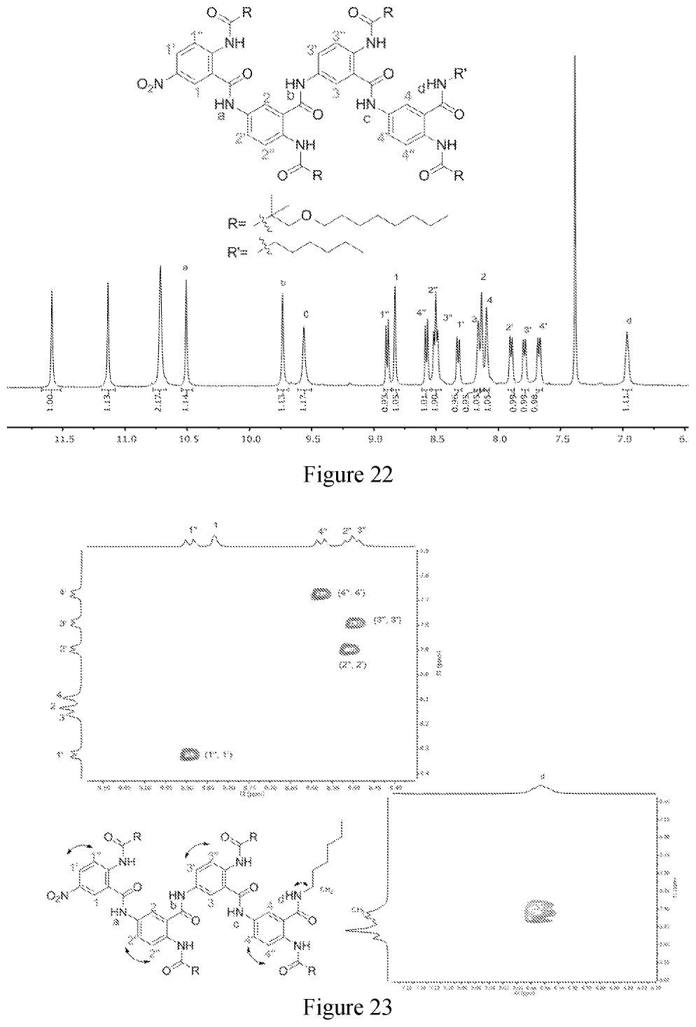

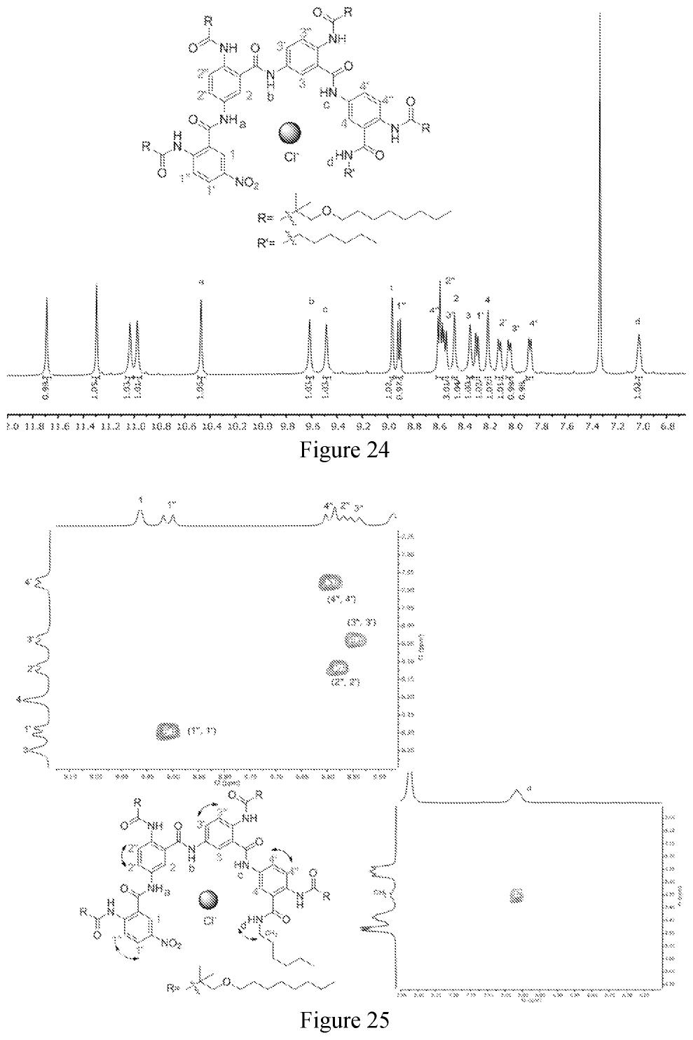

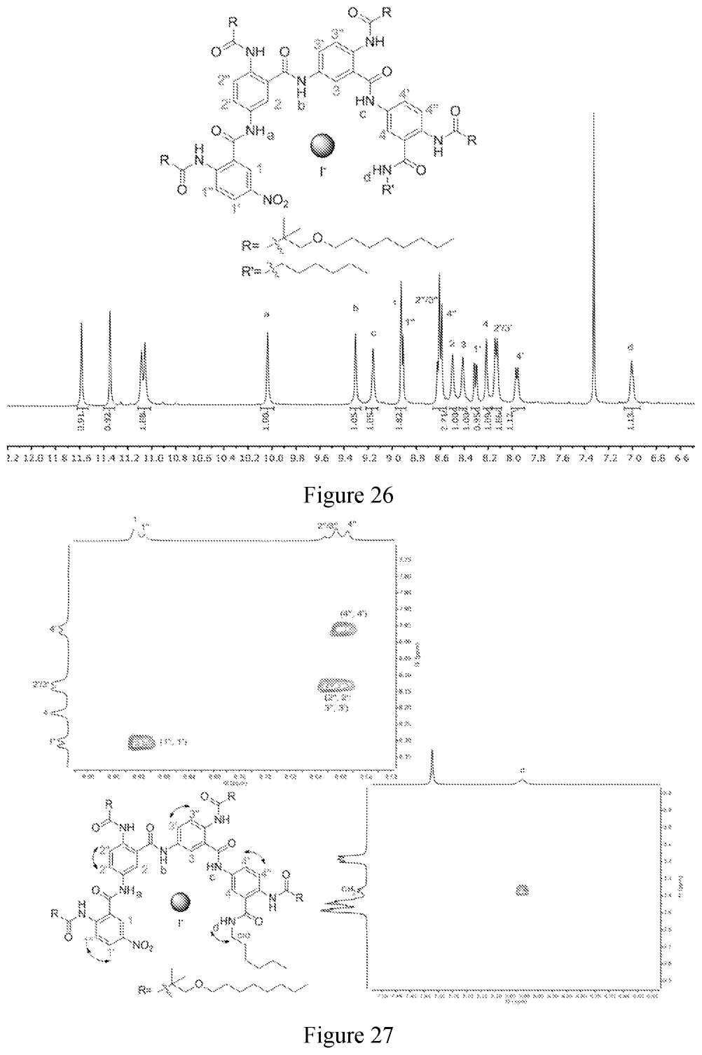

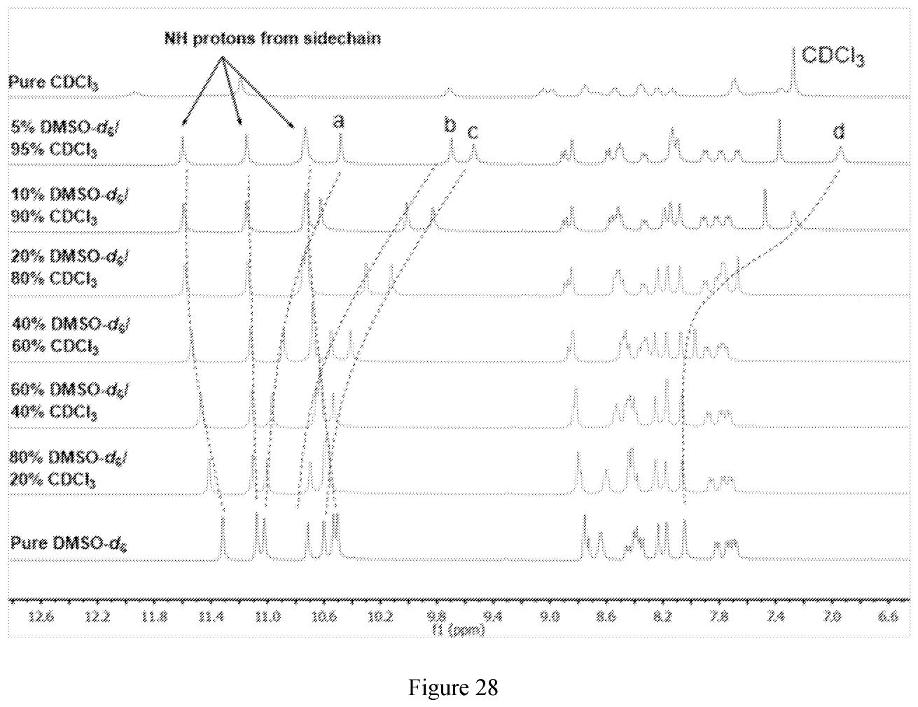

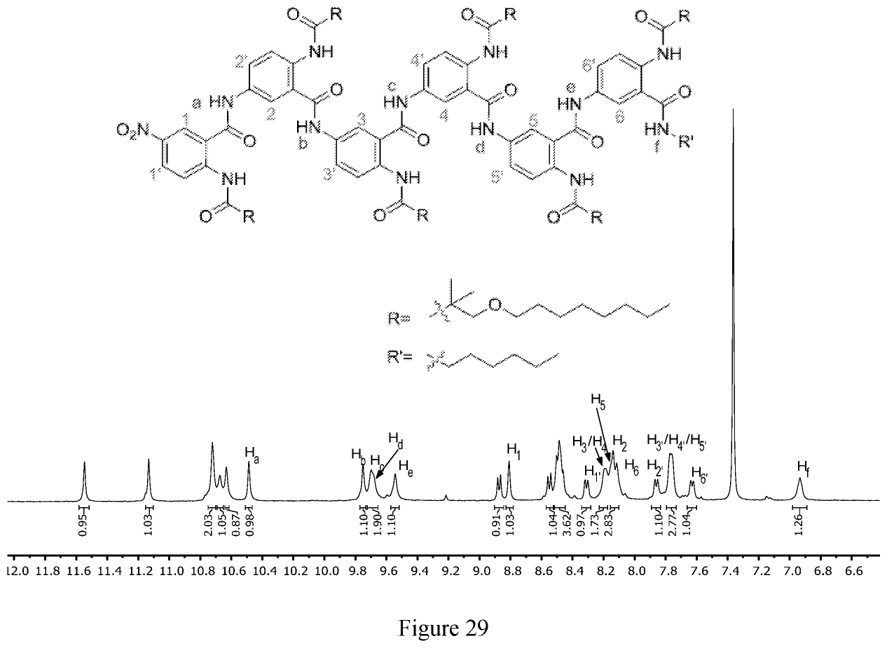

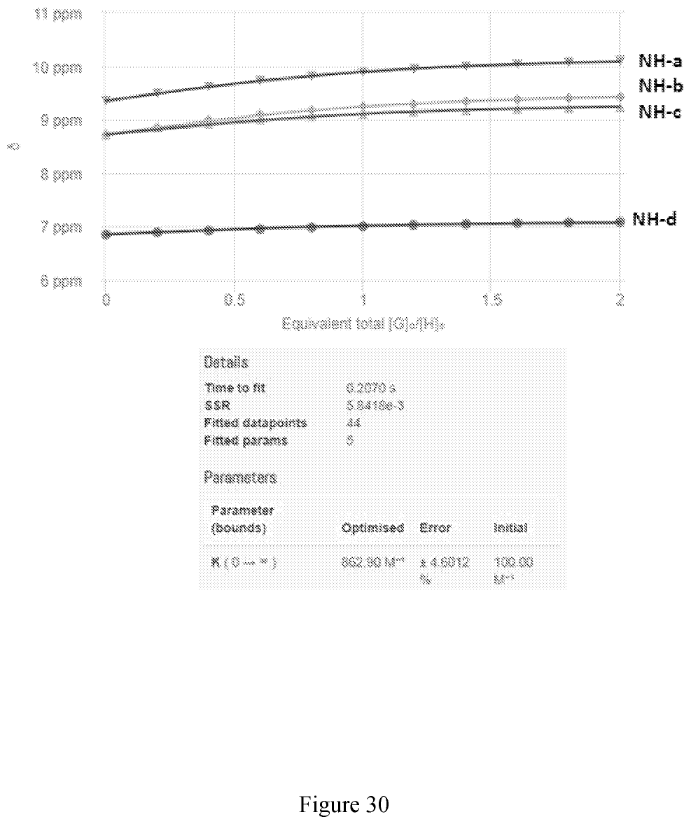

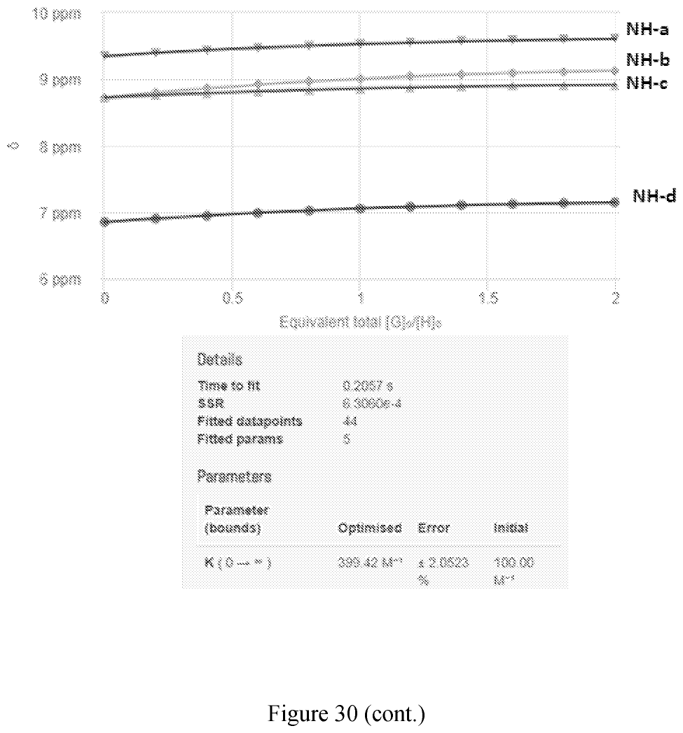

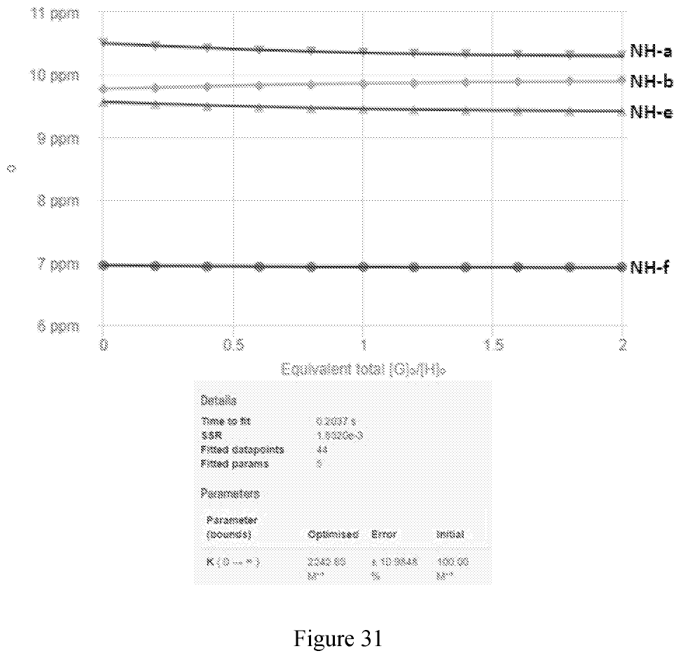

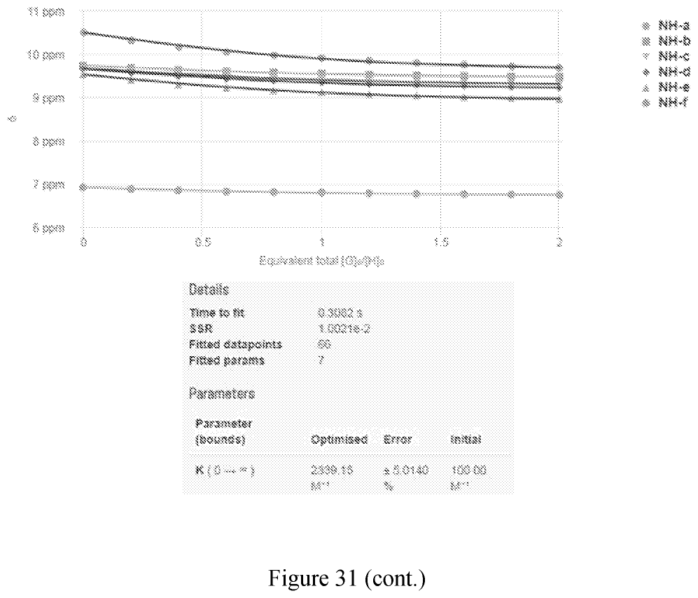

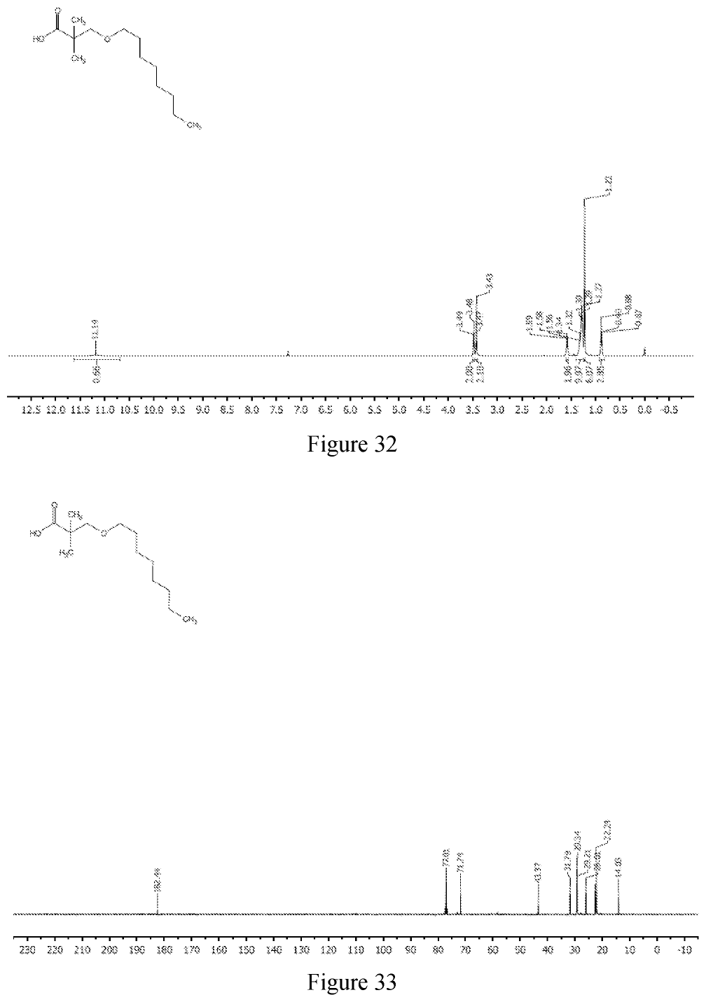

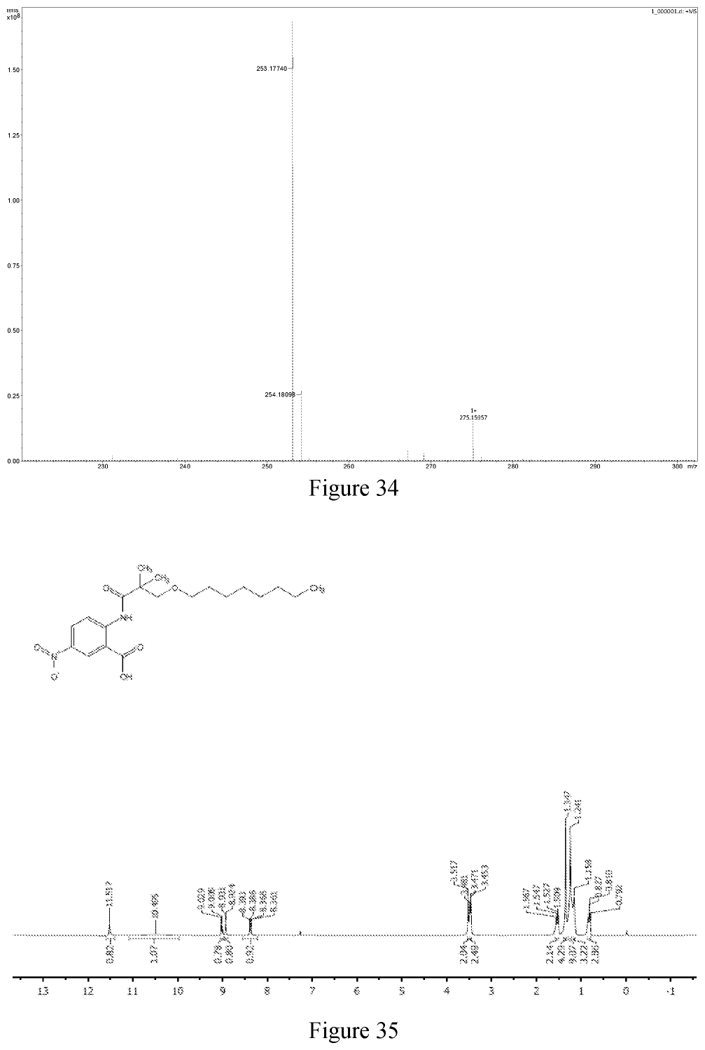

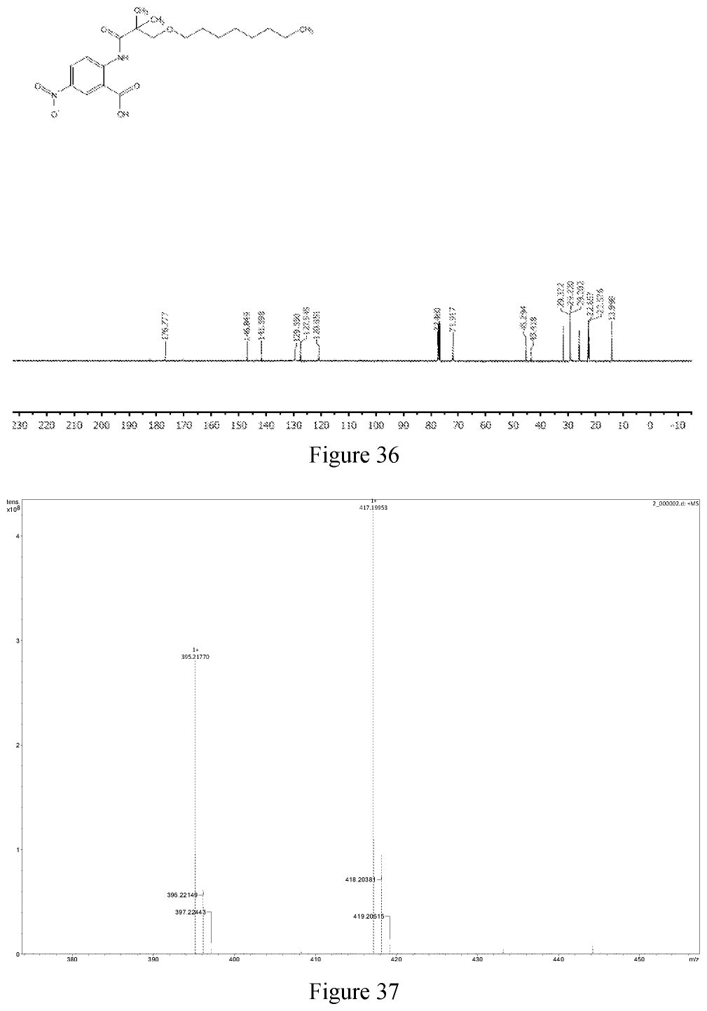

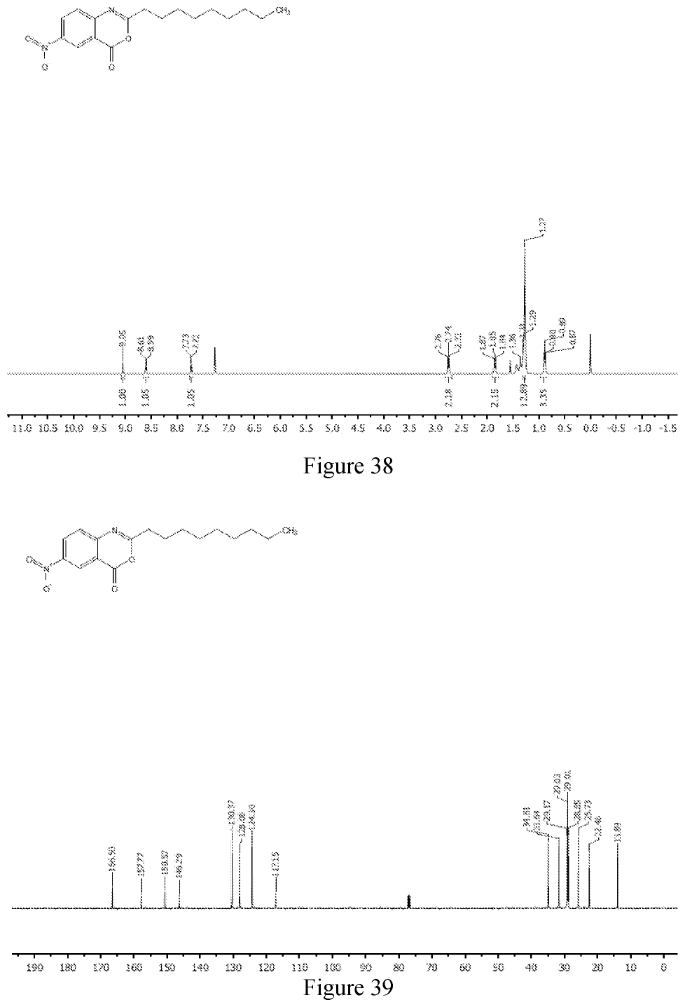

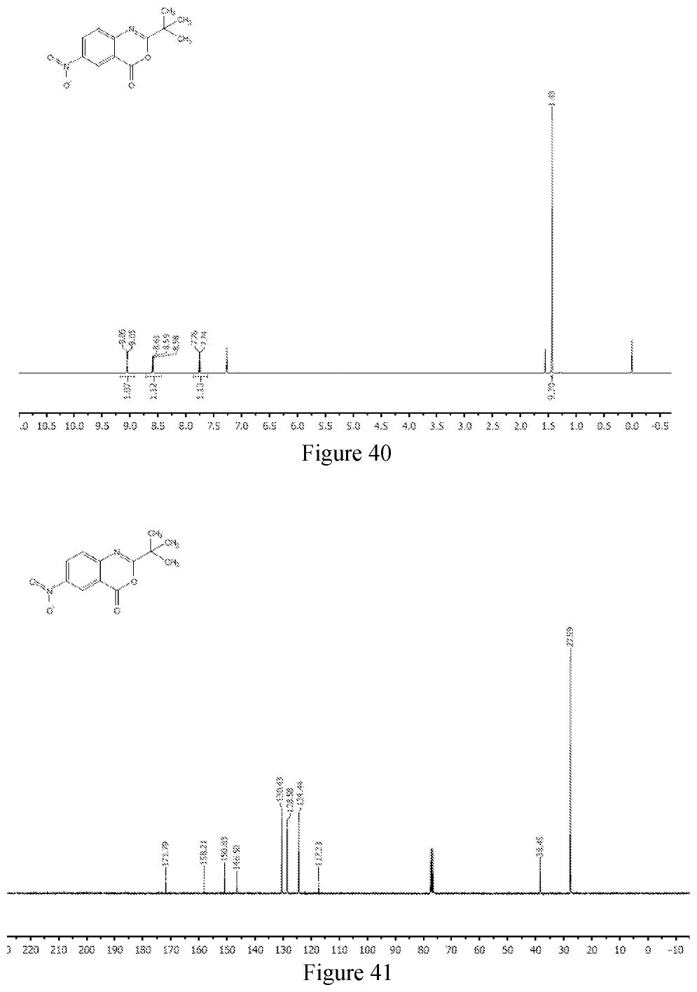

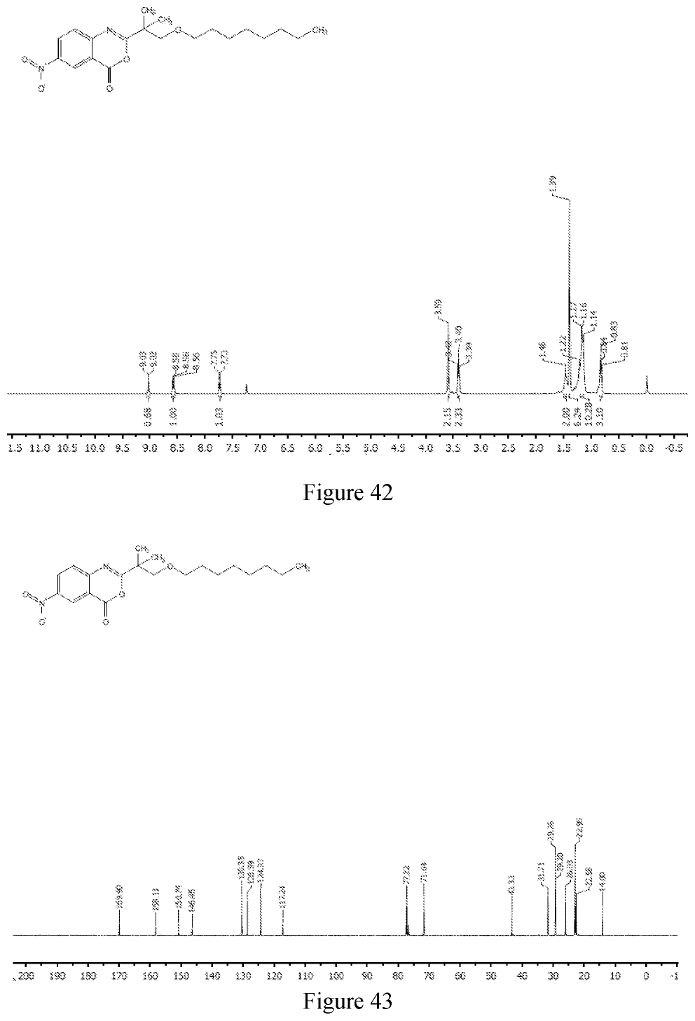

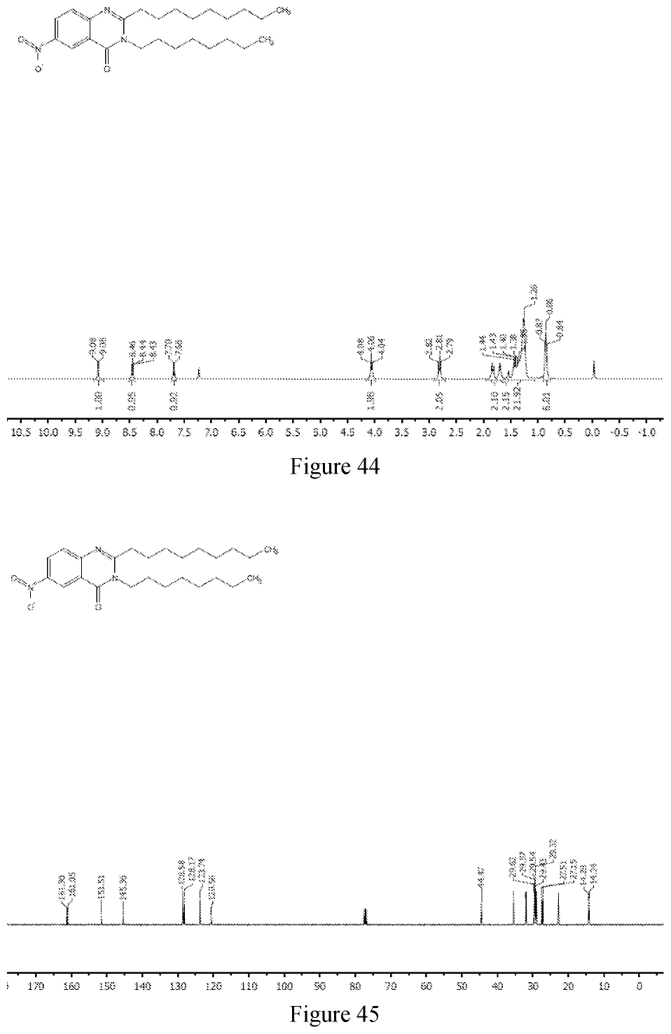

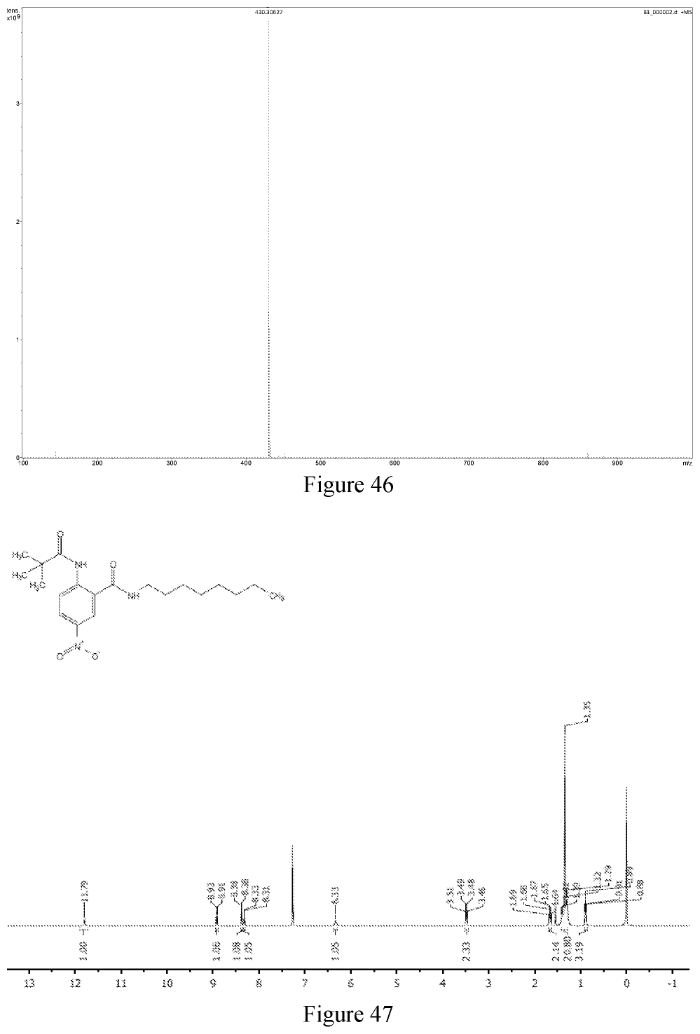

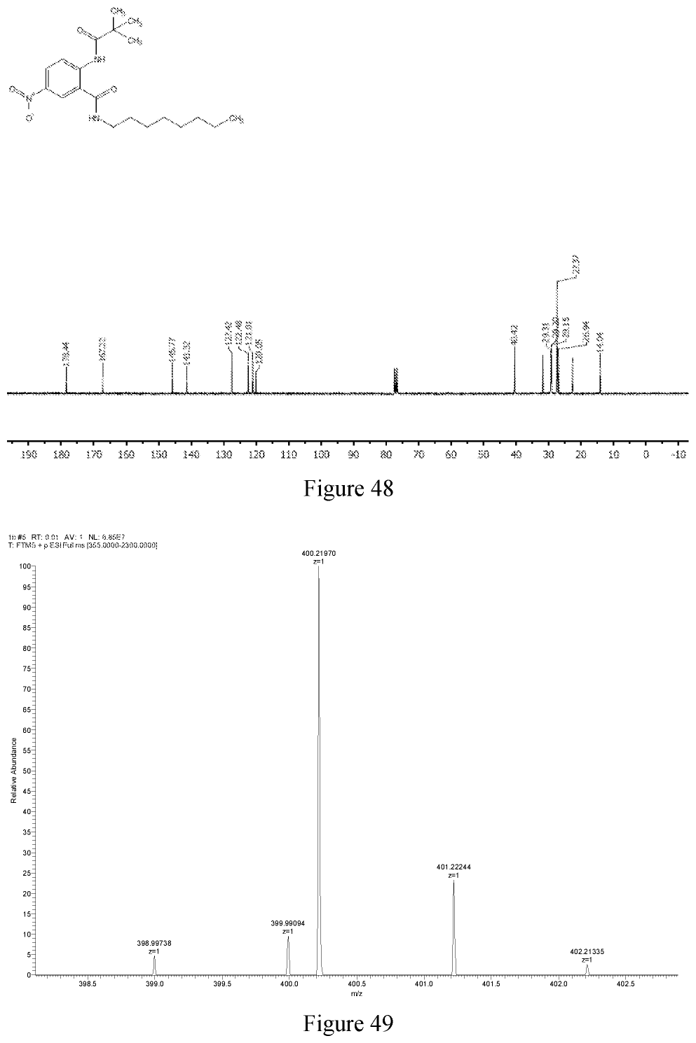

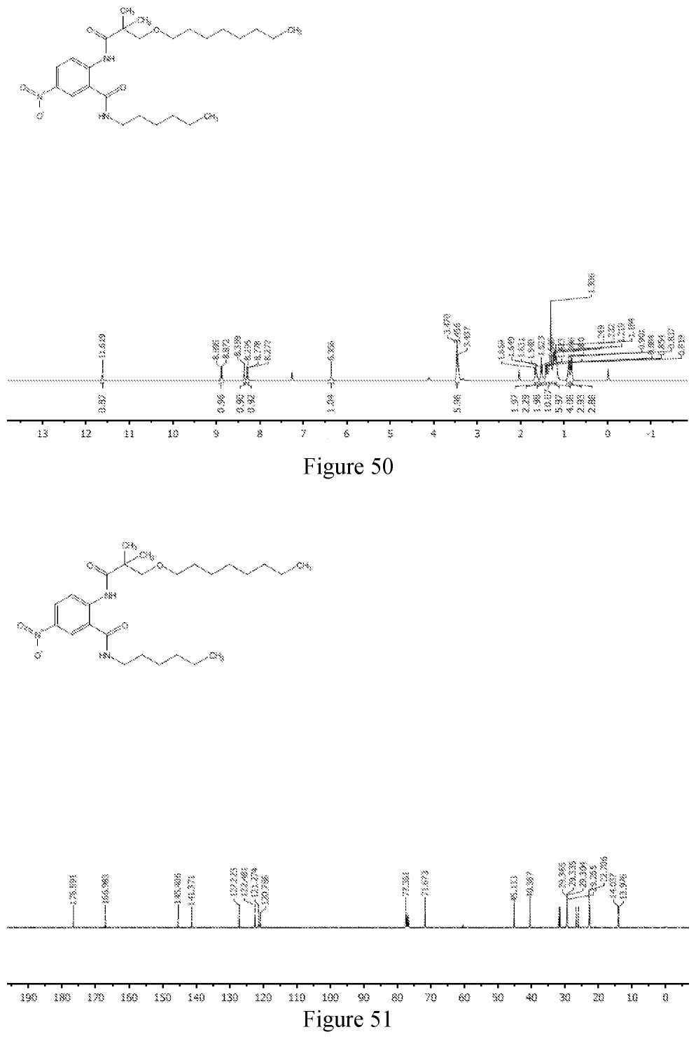

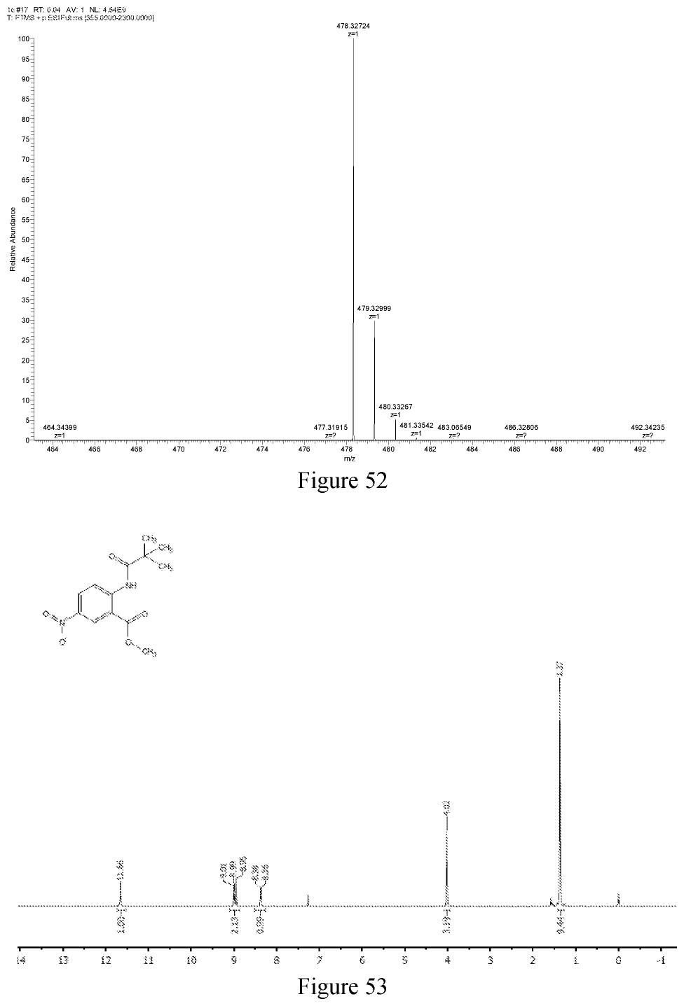

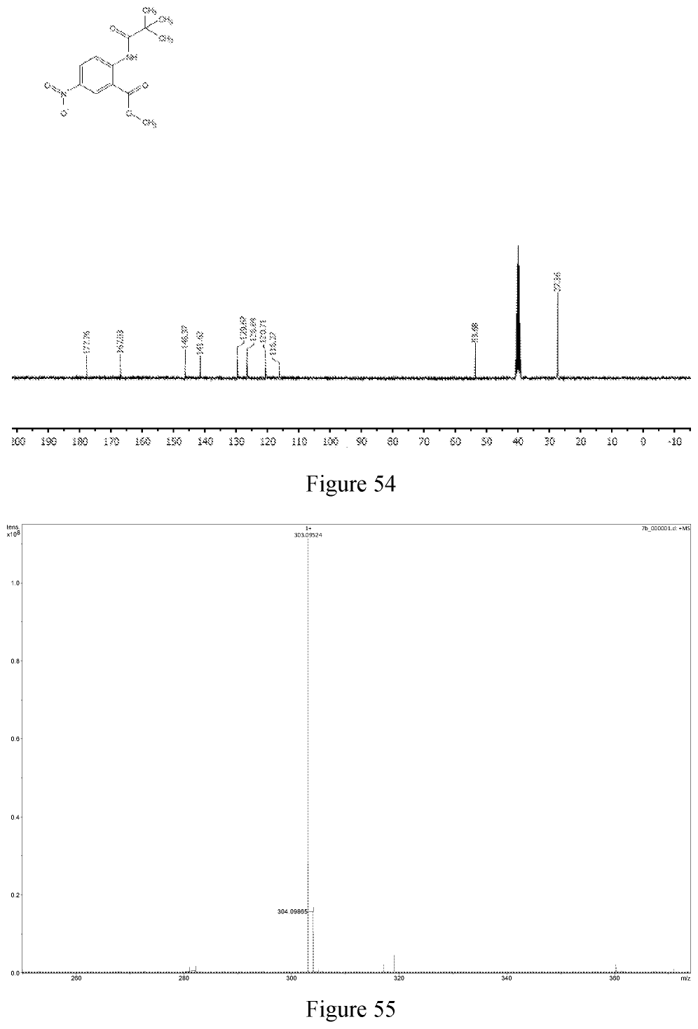

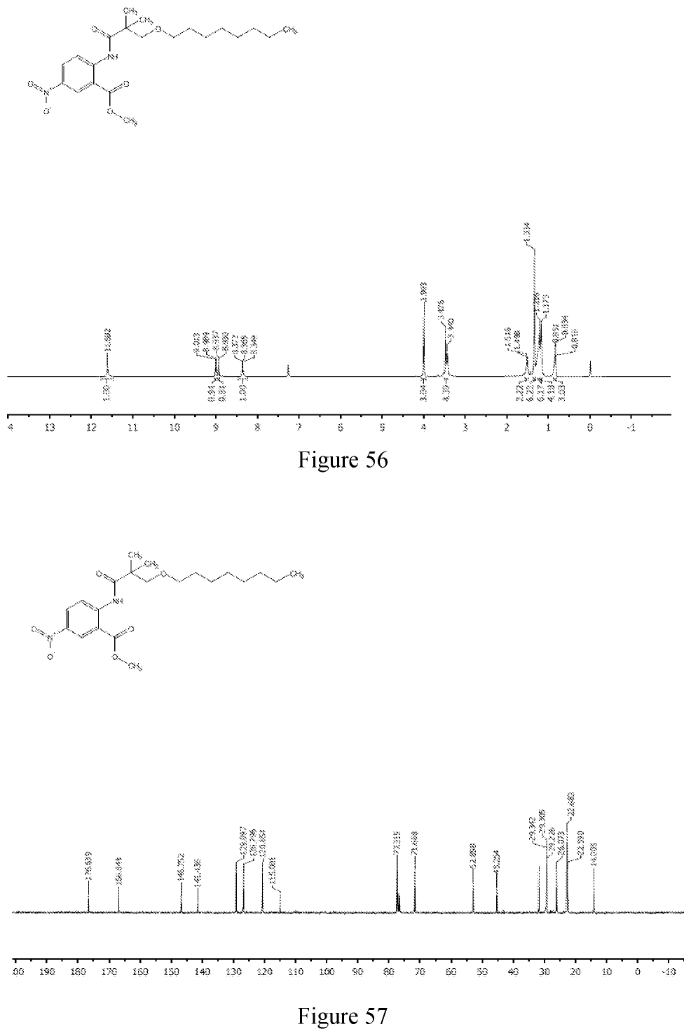

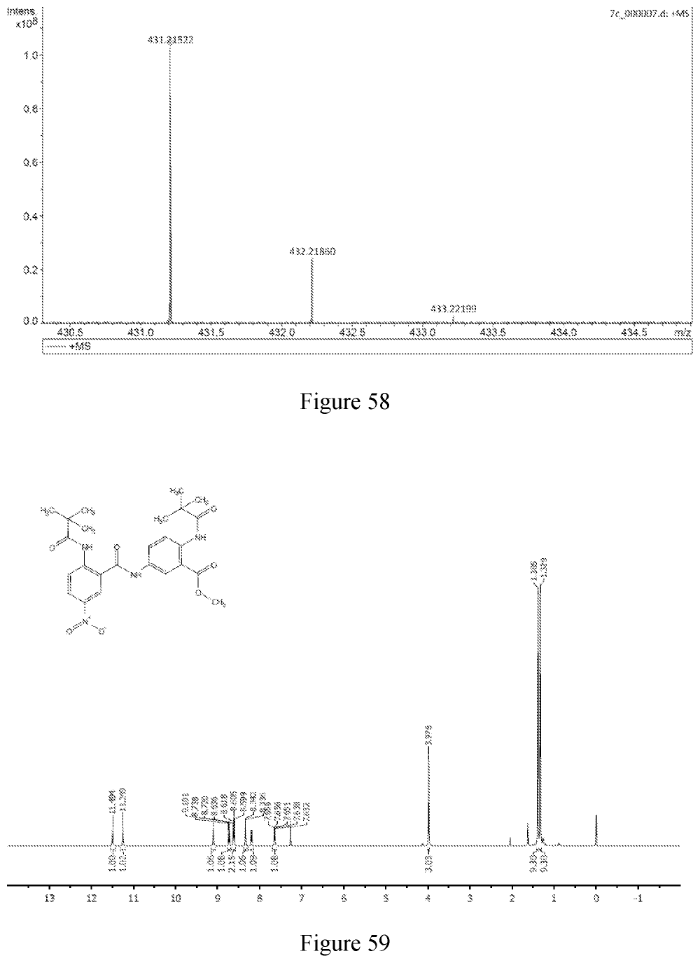

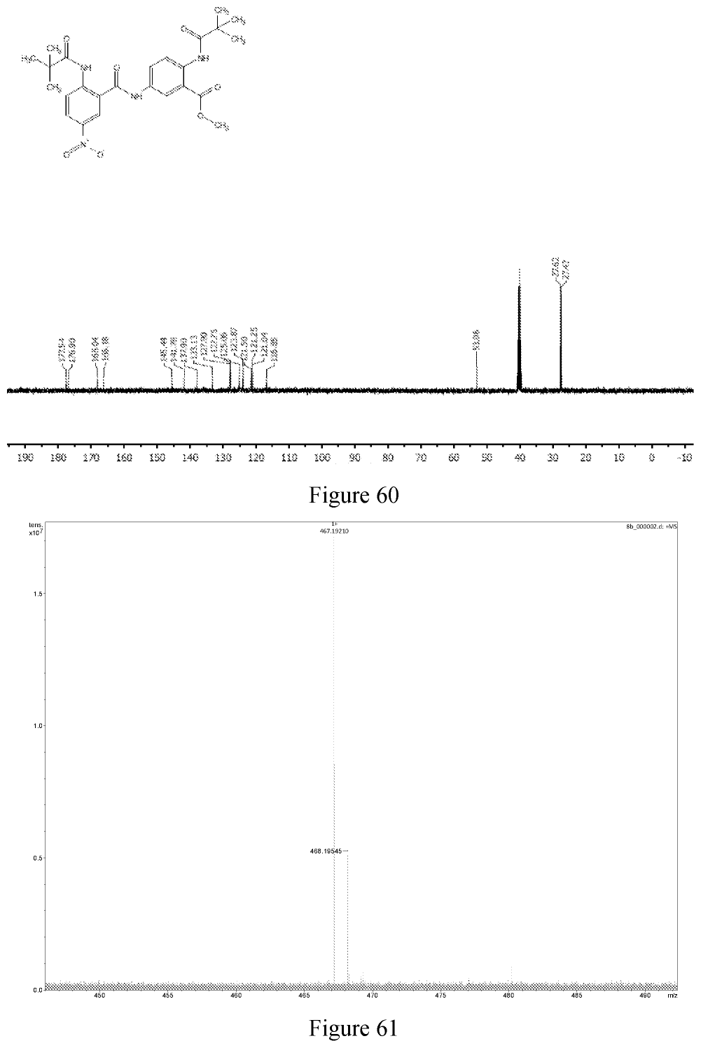

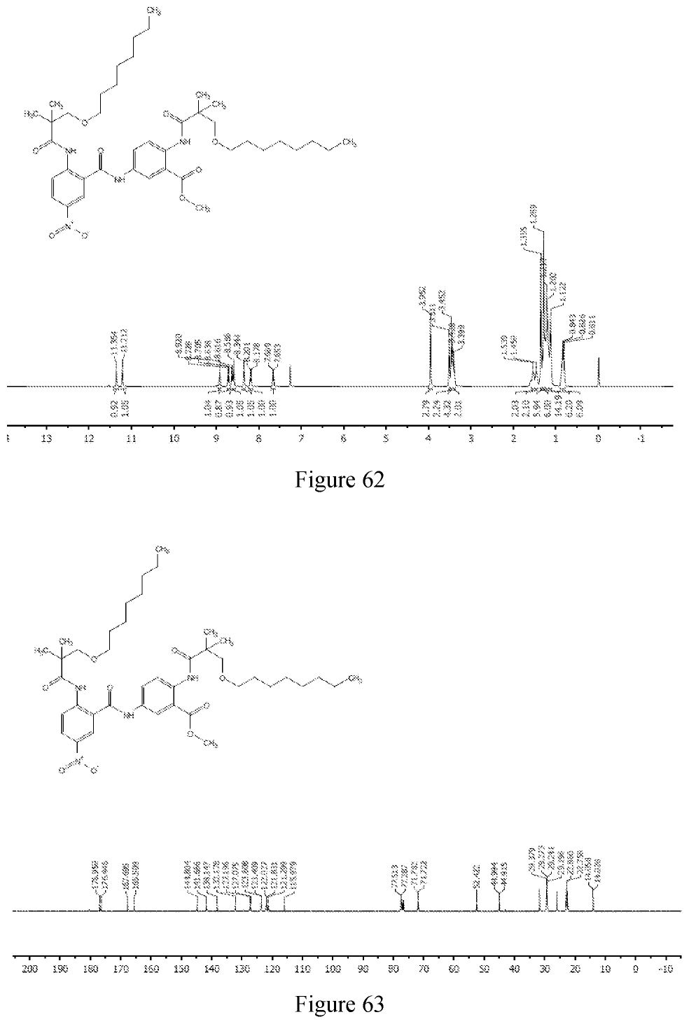

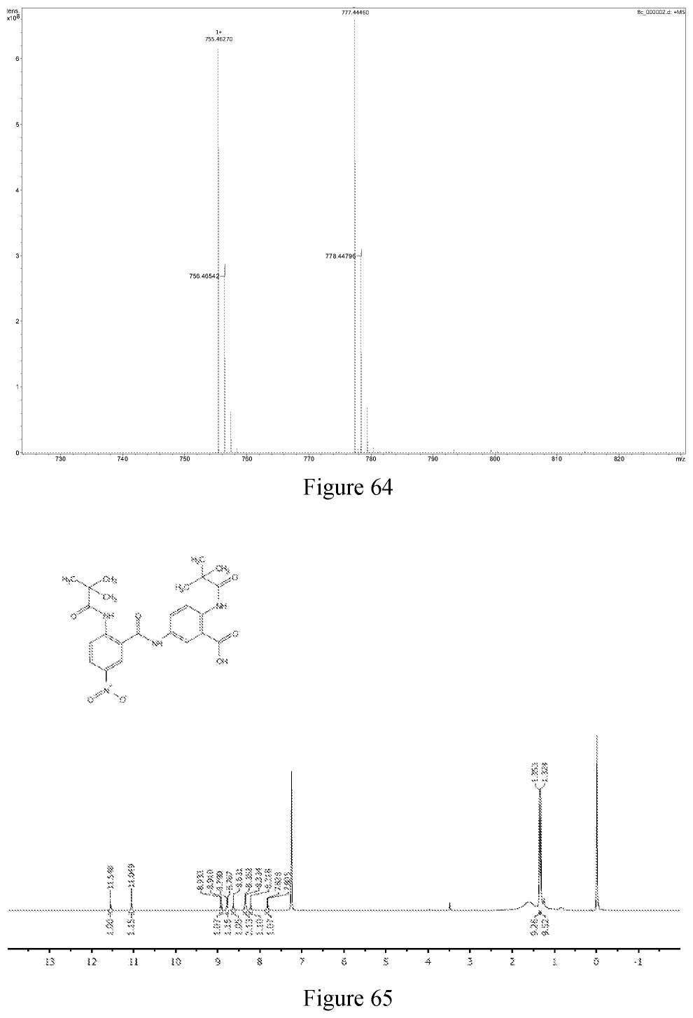

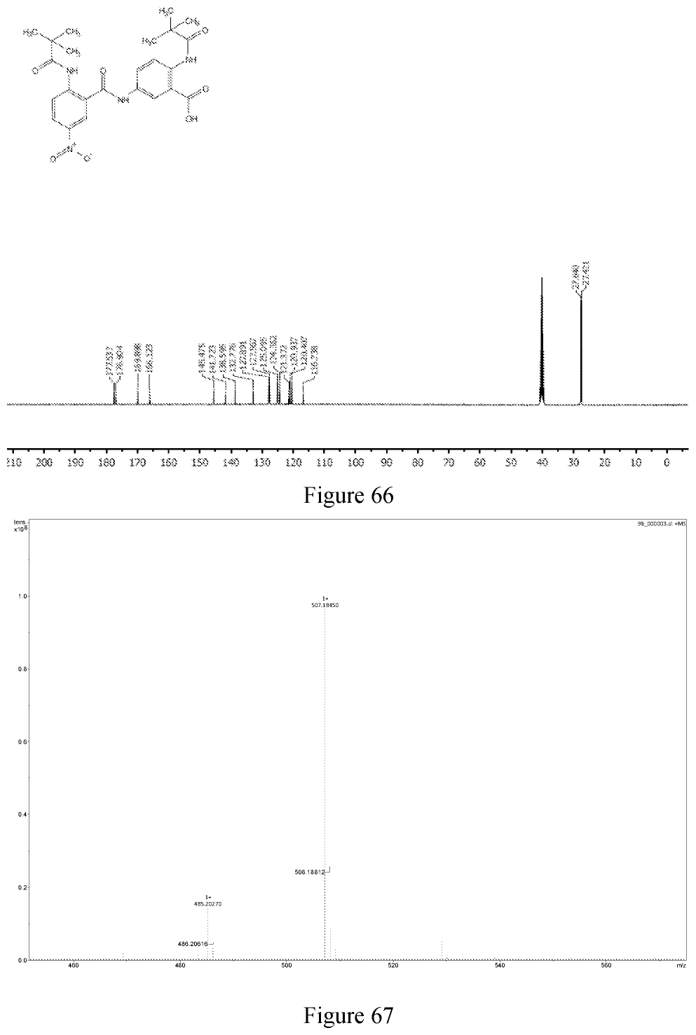

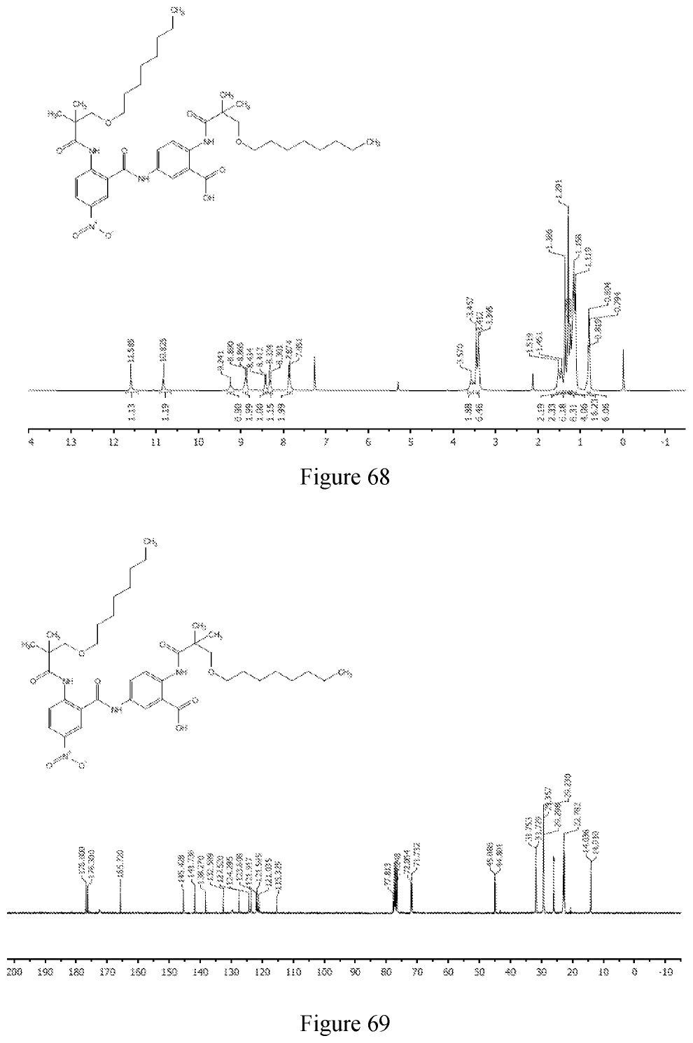

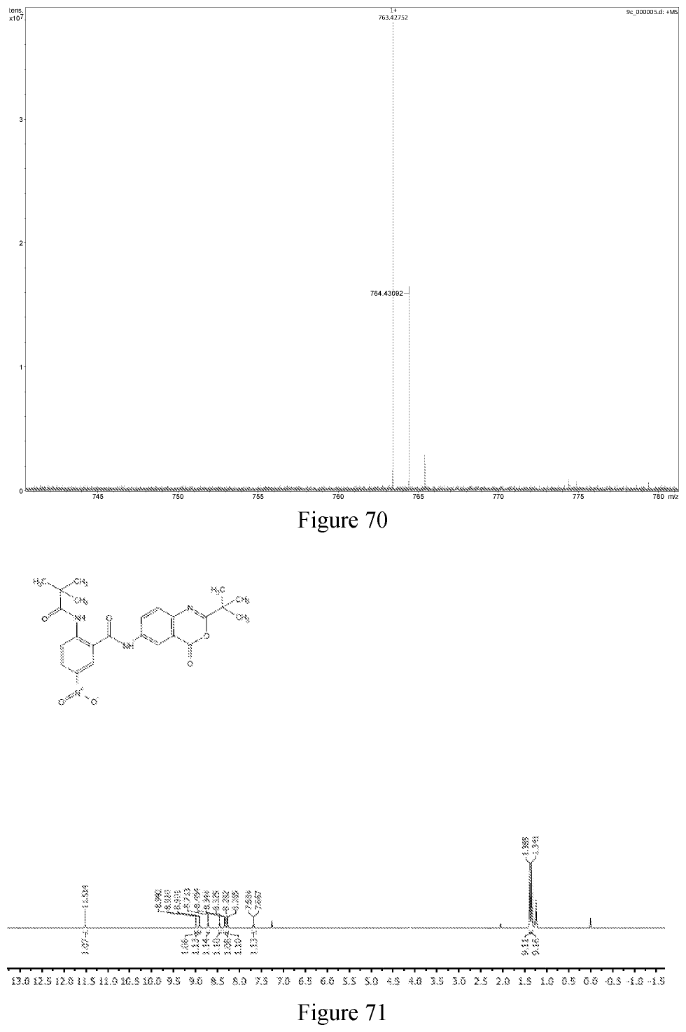

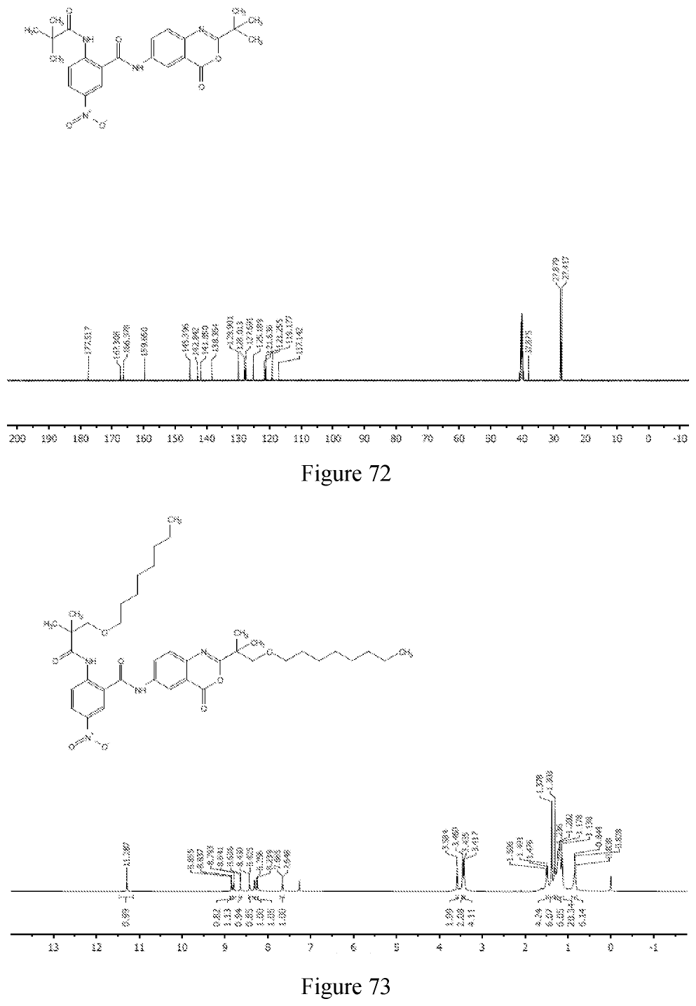

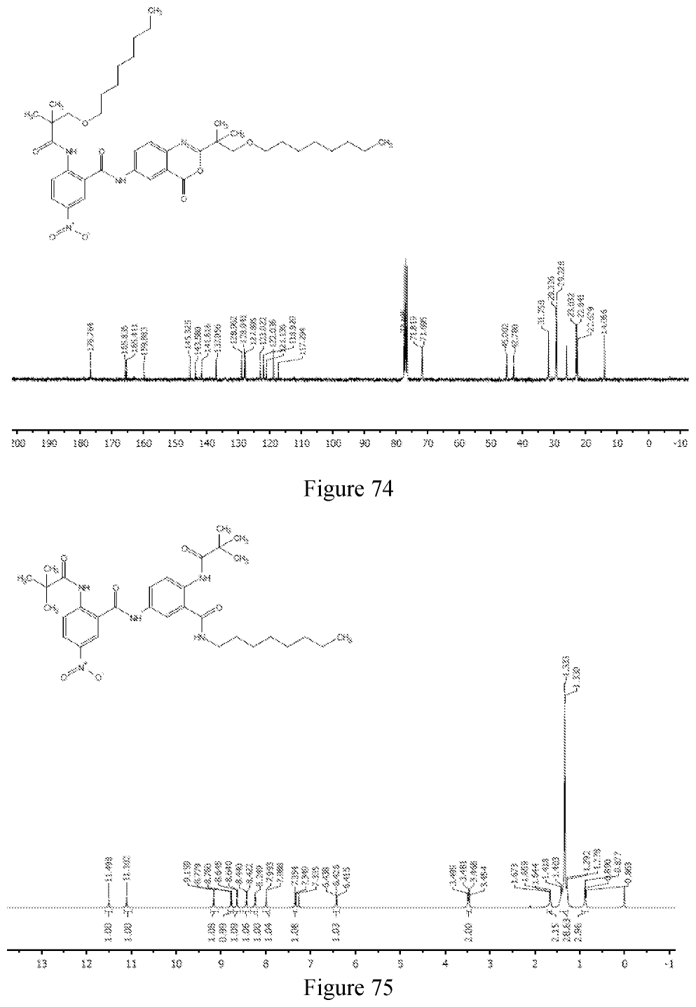

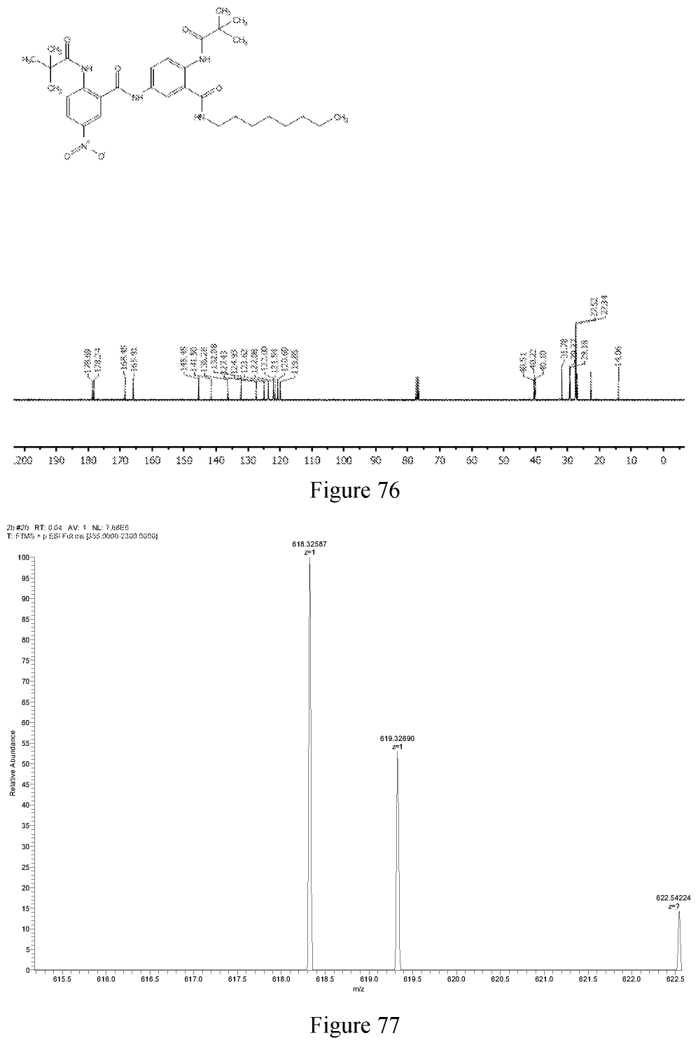

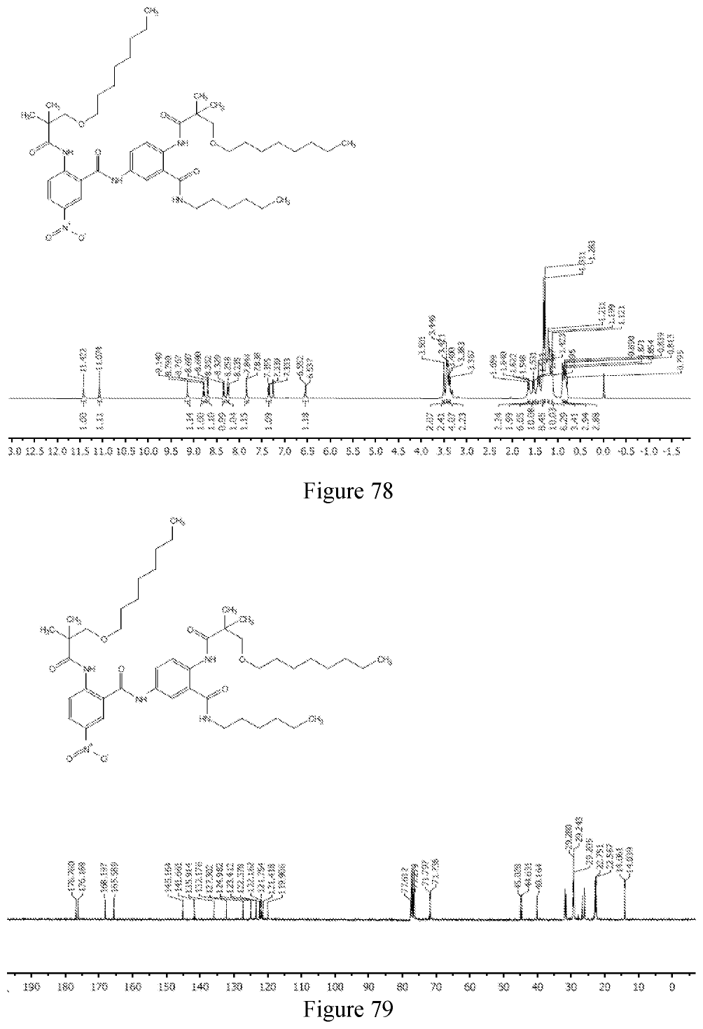

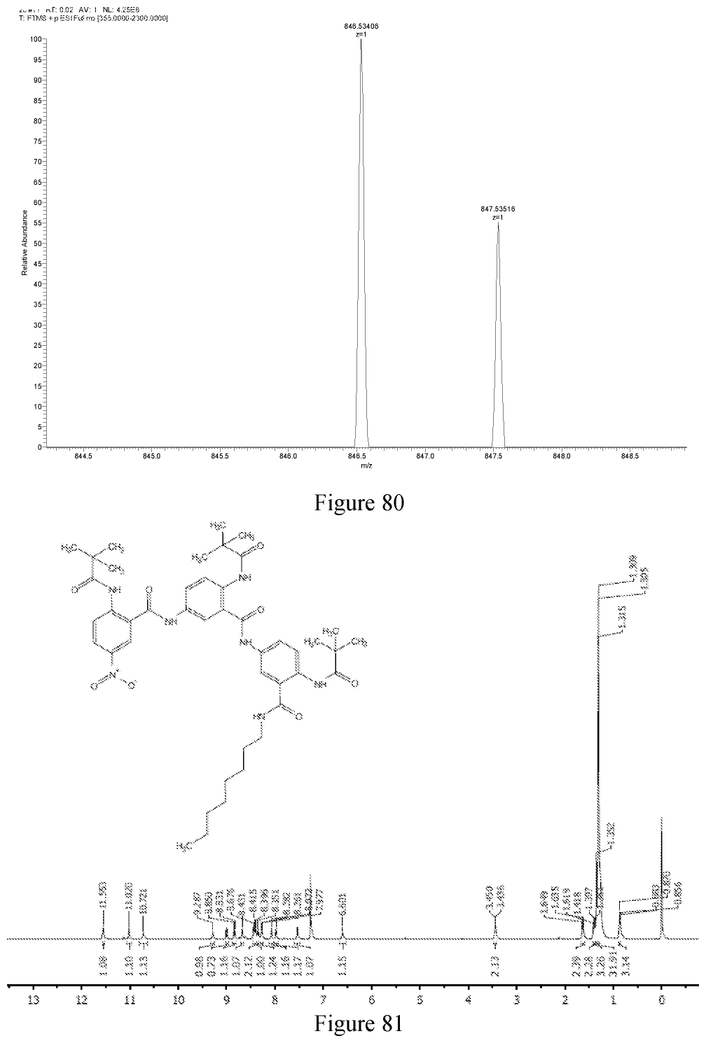

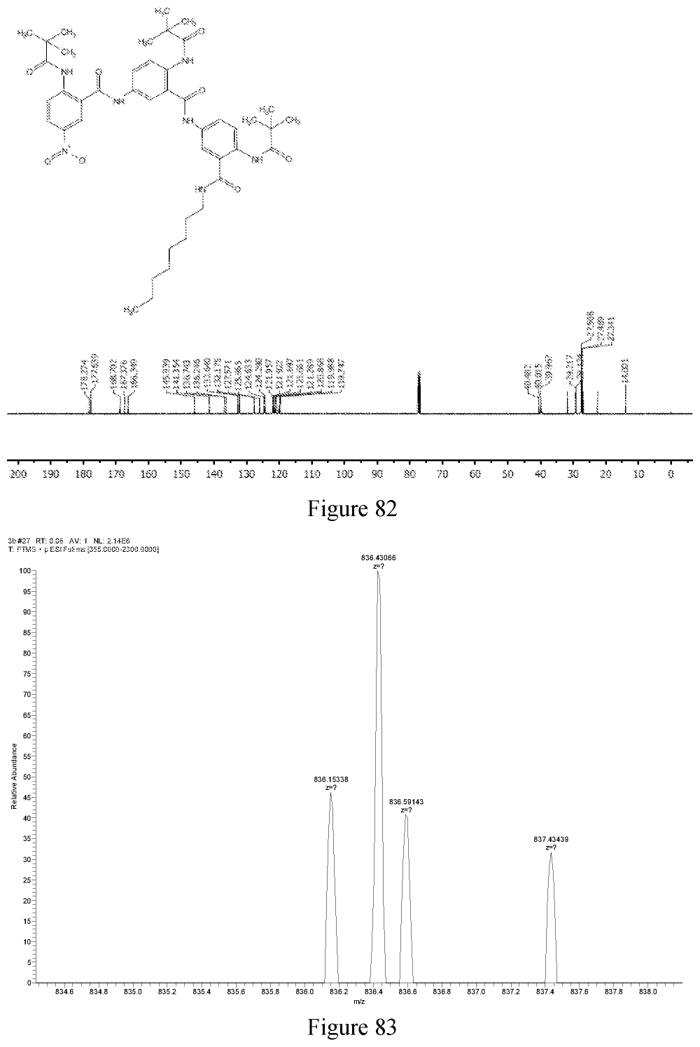

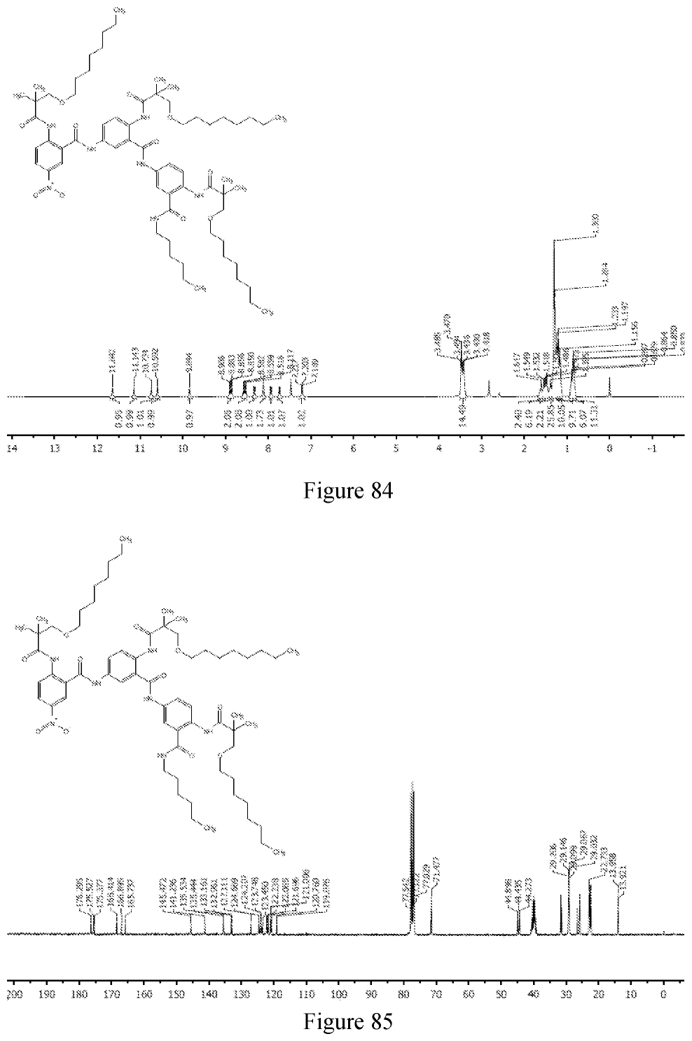

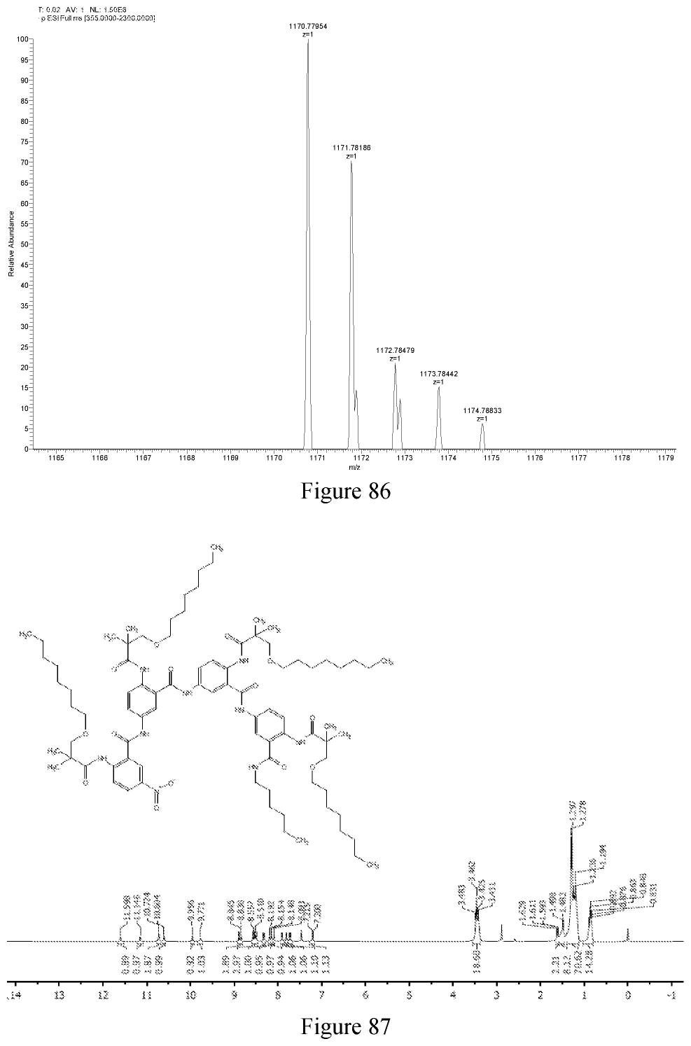

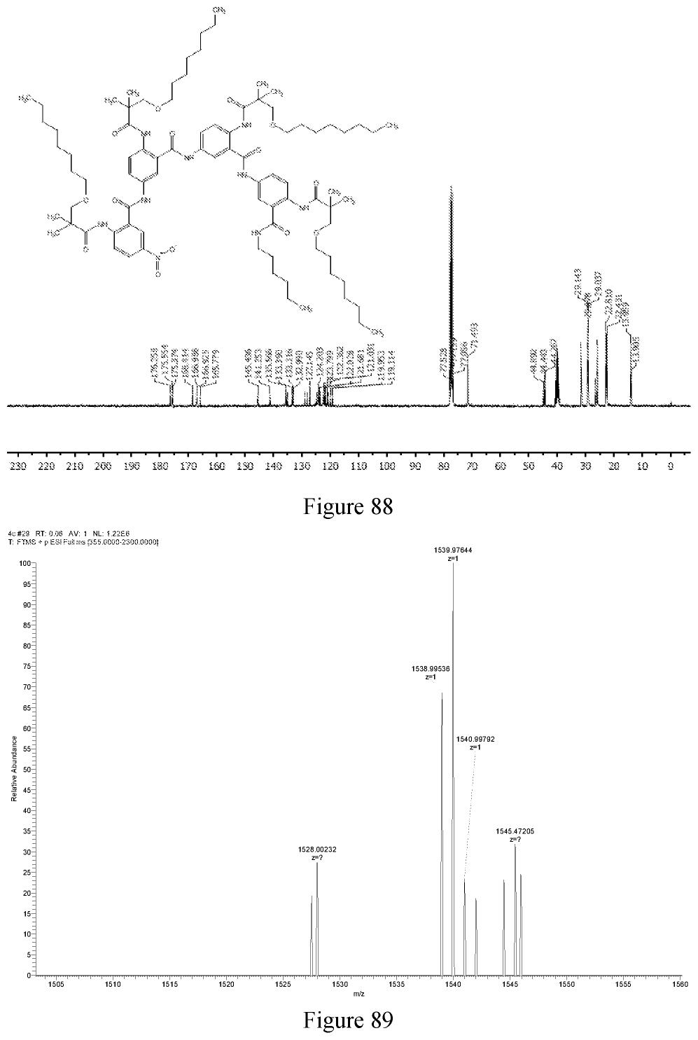

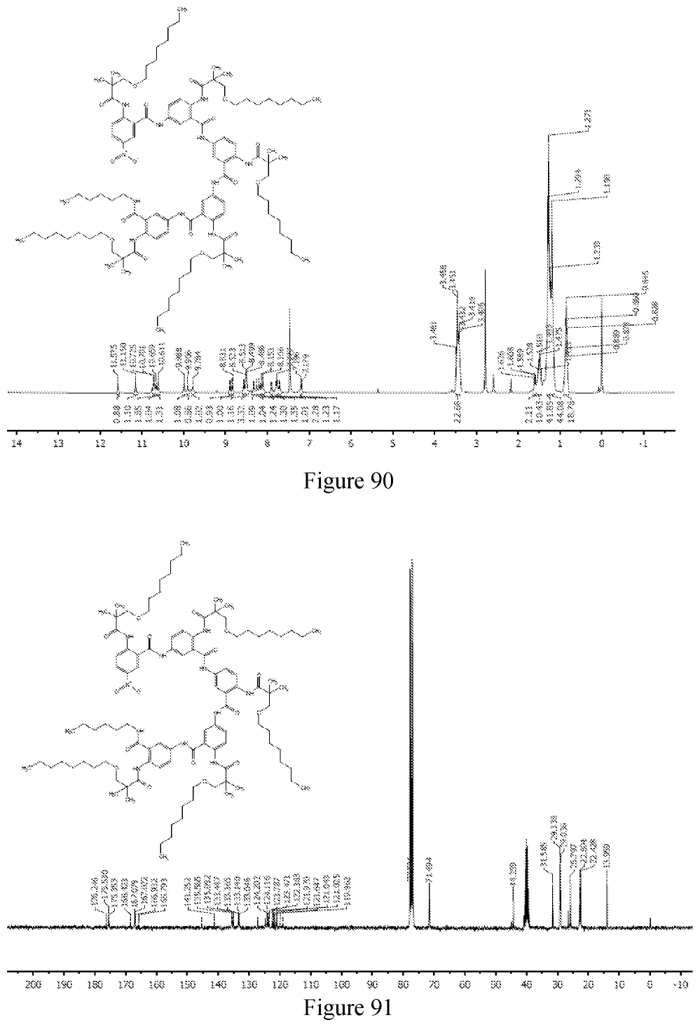

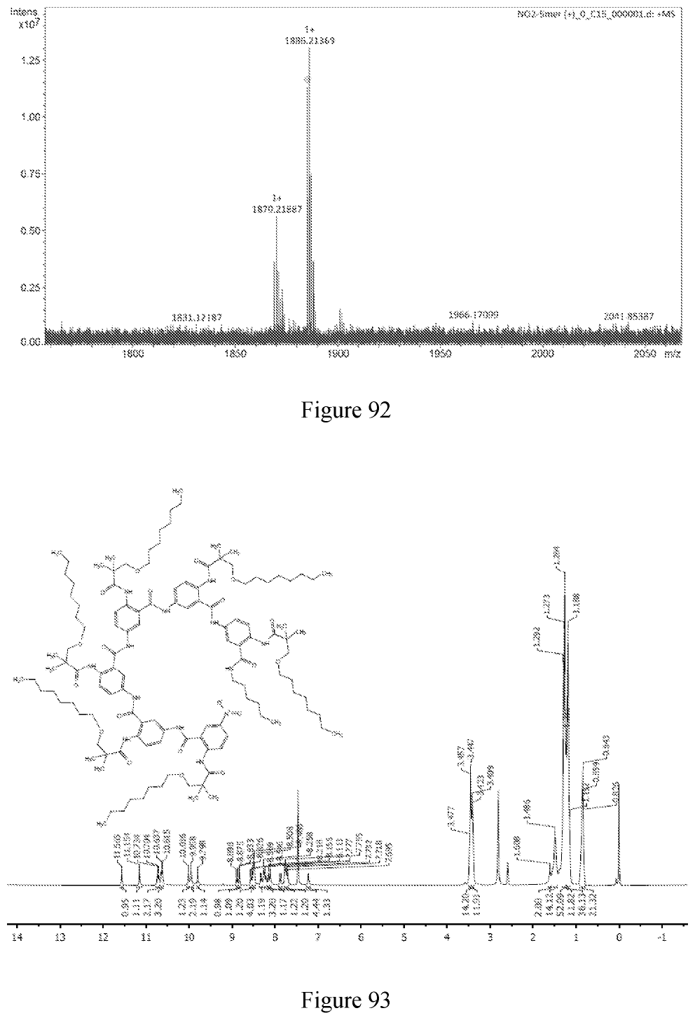

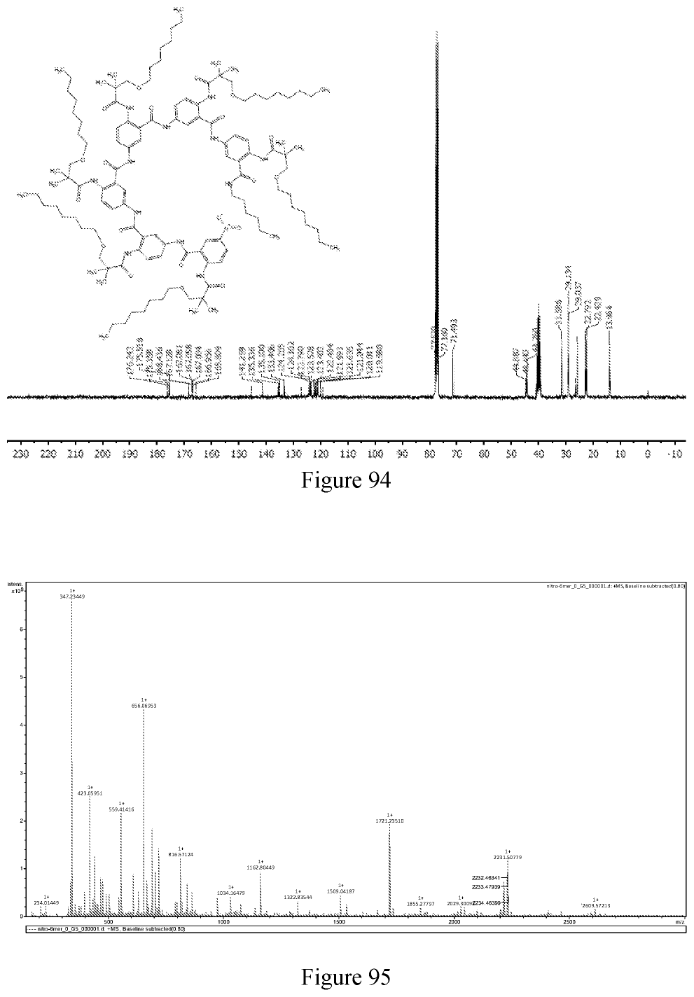

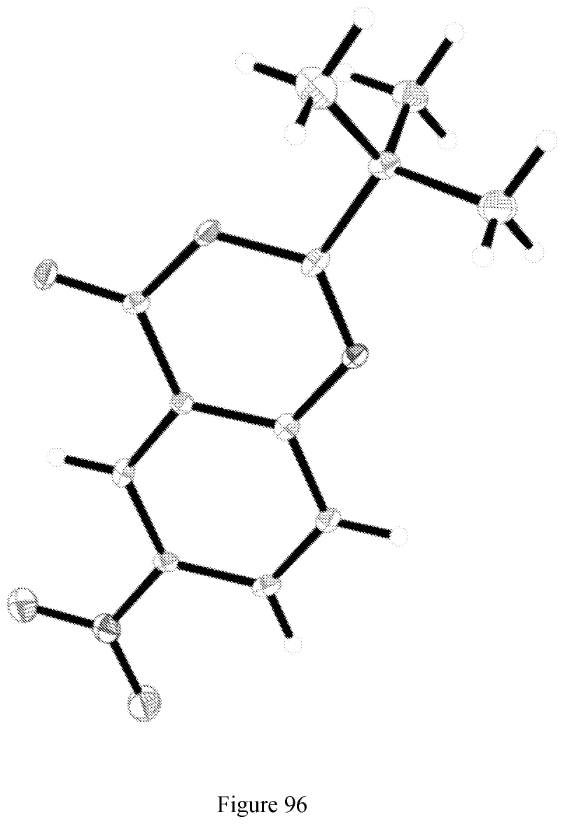

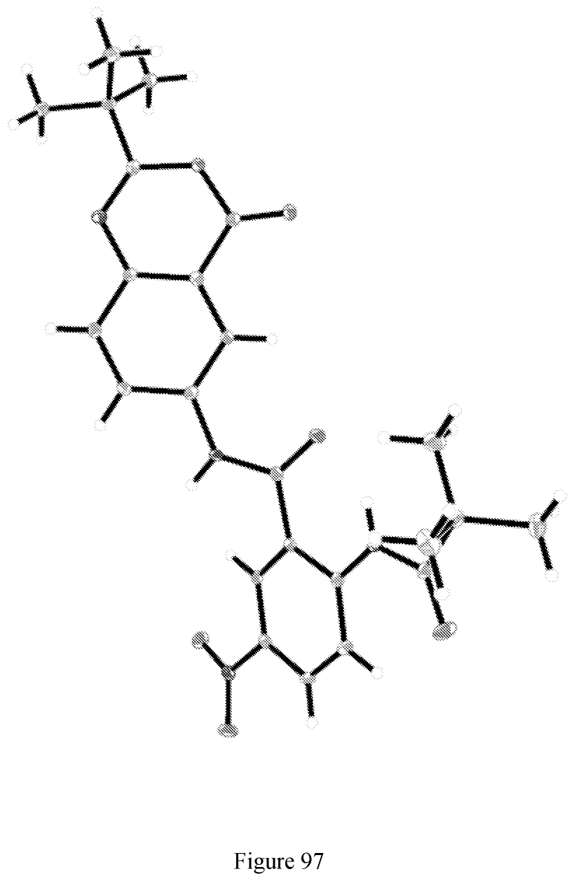

Like that of free 4c, the NOESY spectrum of 4c with 1 equiv. of n-Bu₄N⁺I⁻ (FIG. 4b) also contains NOEs between protons a and 1, b and 2, c and 3, along with d and 4 (FIG. 17), consistent with the presence of the intramolecular H-bonds. The NOEs involving amide protons a, b, and c, and the “outer” proton 2′, 3′, and 4′, which are observed with free 4c, are absent in the presence of n-Bu₄N⁺I⁻. In contrast, a strong NOEs involving each of these amide protons and the “inner” aromatic proton 2, 3, or 4 is detected. Similar to n-Bu₄N⁺I⁻, mixing n-Bu₄N⁺Cl⁻ with 4c also results in significant weakening or disappearance of NOEs involving the backbone amide and outer aromatic protons, while NOEs involving backbone amide protons and “inner” aromatic protons are strengthened (FIG. 18). The observed NOEs between the amide and inner aromatic protons in the presence of iodide or chloride ion indicate that anion-binding and anion-induced folding is a general behavior of 4c and, similarly, its homologous oligoamides.

The fact that only NOEs between amide protons a, b, and c and inner aromatic protons 1, 2, 3, and 4 are evident in the presence of iodide or chloride ion demonstrates that, upon binding an anion, oligoamide 4c adopts a conformation in which its backbone amide NH groups point convergently, i.e., being placed on the same side as the inner aromatic protons. This is consistent with the adoption of the crescent conformation shown in FIG. 4b. The entropic cost for adopting such a conformation is compensated by the enthalpic contribution from the multiple H-bonding interactions between the anion guest and the backbone amide and aromatic protons.

A new, highly efficient amide bond formation strategy has been established. Repetitive coupling of readily available building blocks derived from 5-aminoanthranilic acid led to oligo(5-amino-N-acylaminoanthranilic acids) with defined lengths in high yields. These are hitherto unknown aromatic oligoamides due to the unavailability of methods for their synthesis. The stepwise coupling of monomeric or oligomeric units allows oligoamides of defined lengths to be prepared under simple conditions that involve refluxing without any coupling reagent. Initial studies indicate that the resultant aromatic oligoamides, with multiple amide NH groups, undergo anion-dependent folding. Given their ready synthetic availability and tunable oligomer length, these novel aromatic oligoamides with multiple H-bond donors, provide a new, versatile platform for developing guest-dependent foldamers or binders of anions and other polar guests, with adjustable affinity and specificity.

TABLE 1
Summary of relative intensity of important NOE interactions
between protons of interest. The intensity of NOE interaction
(a, 1) was set to be 1.00 as an internal standard

| | NOE interactions | | | | | | | | | |
|---|---|---|---|---|---|---|---|---|---|---|
| | (a, 1) | (a, 2) | (a, 2′) | (b,2) | (b, 3) | (b, 3′) | (c, 3) | (c, 4) | (c, 4′) | (d, 4) |
| 4c | 1.00 | 2.47 | 0.58 | 5.32 | 4.15 | 1.57 | 5.91 | 0.59 | 0.36 | 0.24 |
| 4c-TBACI | 1.00 | 5.79 | 0.13 | 9.56 | 5.79 | 0.39 | 9.22 | 2.04 | NA | 3.46 |
| 4c-TBAI | 1.00 | 6.40 | NA | 11.73 | 9.19 | NA | 12.70 | 2.96 | NA | 7.48 |

Materials and instruments. Chemicals were purchased from commercial sources and used as received. Silica gel for analytical thin layer chromatography (TLC) and column chromatography (mesh 230-400) were purchased from Sorbent Technologies Inc. ¹H NMR spectra were recorded at 400 MHz on Bruker-400 and 500 MHz on Bruker-500, ¹³C NMR spectra were recorded at 75 MHz on Bruker-300 spectrometers, at ambient temperature using CDCl₃ or DMSO-d₆ as solvents (Cambridge Isotope Laboratories, Inc.). Chemical shifts are reported in parts per million (ppm) downfield from TMS (tetramethylsilane) or the deuterated solvents. Coupling constant in ¹H-NMR were expressed in Hertz (Hz). Regular mass spectra (MS-ESI) were recorded on a Thermo Finnegan LCQ Advantage MS spectrometer. High-resolution electrospray ionization mass spectra (HRMS-ESI) and Matrix-assisted laser desorption/ionization (HRMS-MALDI) were recorded on a Bruker SolariX 12 T Fourier Transform Mass Spectrometer.

Unless otherwise specified, all solvents, including high boiling point solvents, were removed under vacuum with a rotary evaporator. Anhydrous toluene was applied for the coupling reactions.

Preparation of 4-(N, N-dimethylamino) pyridine hydrochloride salt for the amide coupling reactions. 4-(N,N-dimethylamino) pyridine hydrochloride salt (DMAP HCl) was prepared via the published method without any modifications. For DMAP HCl mediated-ring opening reaction of benzoxazinone derivatives, 20 mol % of DMAP HCl and anhydrous toluene were applied to coupling reactions. The reaction suspended solution containing equimolar amounts of benzoxazinone derivatives and amine and 20 mol % DMAP HCl was heated under reflux with an oil bath. The reaction progress was always monitored with TLC plates. Upon the complete consumption of starting material, the workup was carried out which could be different, largely depending on properties of target molecules.

Hydrogenation reaction. The reduction of nitro compounds to their corresponding amine were carried out in a mixed solvent of 20% methanol and 80% CH₂Cl₂, in the presence of a catalytic amount of Pd/C and pressured hydrogen gas. The resulting reaction solution was stirred at room temperature for 2-8 hours. Upon the completion of reduction reactions, developed TLC plates were stained with a solution of ninhydrin in ethanol and the spot of aromatic amines always turned to be from red to purple. Pd/C was then filtered out and the filtrate was concentrated to afford the corresponding amines which were pure enough and applied directly to the coupling reaction without any further purifications. No analytical data is available for aromatic amines, as they were typically considered to be unstable under the ambient environment.

¹H NMR Titration methods. Titrations of 4c and 6c with anions as their tetra-n-butyl ammonium (TBA) salts were carried out by ¹H NMR (400 MHz on Bruker-400) at room temperature (298 K). 0.5 mL of receptor 4c (5.0 mM) or 6c (2.0 mM) were prepared in the rubber-cap NMR tubes. Aliquots of concentrated TBACl or TBAI solution (50 mM for the titration of 4c and 20 mM for the titration of 6c) containing the same amount of receptors were added into the receptor solution using 20 microliter pipette. An initial ¹H NMR spectrum was collected and additional spectra were obtained after each injection of TBACl or TBAI solution. All titration ¹H NMR spectra were stacked together and the chemical shifts of amide protons were then fitted to the proposed 1:1 binding model using BindFit v0.5 (http://app.supramolecular.org/bindfit/).

Synthesis and Characterization

Synthesis of Benzoic Acid.

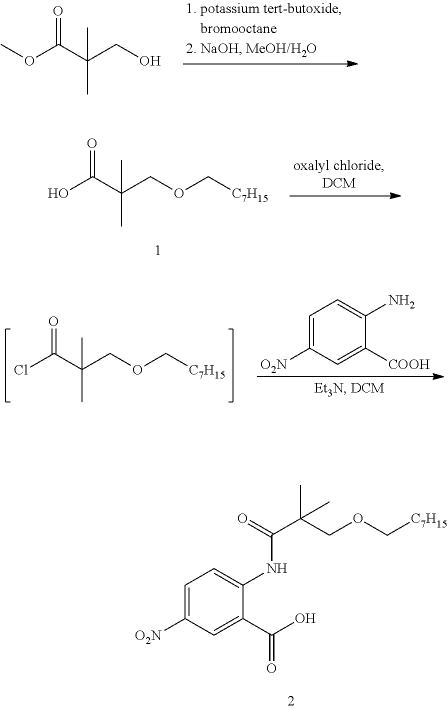

Compound 1: To a 250 mL of round flask were added bromooctane (26.4 g, 200 mmol) and methyl 3-hydroxy-2,2-dimethylpropanoate (38.4 g, 200 mmol). The mixture was stirred vigorously at 0° C. with an ice bath, while potassium t-butoxide (24.7 g, 220 mmol) was added portionwise into the mixture. The reaction mixture was heated to 40° C. overnight. The reaction mixture was poured into icy water and aqueous solution was extracted with ethyl acetate (300 mL). The organic layer was then washed with saturated brine (100 mL×2 times) and dried over anhydrous Na₂SO₄. Ethyl acetate was removed and the resulting residue was dissolved in methanol (250 mL) to give a clean solution to which an aqueous NaOH solution (32 g, 0.8 mol in 50 mL of H₂O) was added dropwise. The reaction solution was stirred at room temperature overnight. Methanol was removed and the resulting aqueous solution was extracted with ethyl acetate (300 mL). The organic layer was acidified with dilute HCl aqueous solution and washed with saturated brine (100 mL×2 times) solution. The organic layer was then dried over anhydrous Na₂SO₄ and removed under vacuum to afford pure acid 1 as a colorless oil (41.2 g, 88% for two steps). ¹H NMR (500 MHz, CDCl₃): δ 11.19 (broad, 1H), 3.48 (t, J=5.0 Hz, 2H), 3.47 (s, 2H), 1.56 (m, 2H), 1.29 (m, 10H), 1.22 (s, 6H), 0.88 (t, J=7.5 Hz, 3H). ¹³C NMR (75 MHz, CDCl₃): δ 182.4, 77.0, 71.7, 43.3, 31.7, 29.3, 29.2, 26.0, 22.6, 22.2, 14.0. HRMS (ESI) m/z: [M+Na]⁺ Calcd for C₁₃H₂₆NaO₃ 253.1780; Found 253.1774.

Compound 2: The preparation of corresponding acid chloride from acid 1 was accomplished by adding oxalyl chloride (9.3 g, 73.2 mmol) to a solution of acid 1 (16 g, 69.5 mmol) in dry CH₂Cl₂ (120 mL) followed with a drop of dry DMF as an initiator. The resulting reaction solution was stirred at room temperature for 6 h before removing CH₂Cl₂. The freshly prepared acid chloride was applied to the next step without any purification. To a solution of 2-amino-5-nitro benzoic acid (12.0 g, 66.0 mmol) and triethylamine (8.63 g, 79.2 mmol) in CH₂Cl₂ (150 mL) was added the freshly prepared acid chloride (16.9 g, 68.0 mmol) dropwise. The reaction solution was stirred at room temperature overnight. Then, the organic layer was washed with saturated NaHCO₃ aqueous solution (50 mL×2 times), brine (50 mL×2 times) and diluted HCl solution (50 mL), and dried over anhydrous Na₂SO₄. The concentration of the reaction solution afforded the pure acid 2 (20.6 g, 79%) as a yellowish oil. ¹H NMR (400 MHz, CDCl₃): δ 11.51 (s, 1H), 10.49 (broad, 1H), 9.02 (d, J=9.6 Hz, 1H), 8.93 (d, J=2.8 Hz, 1H), 8.39 (dd, J=9.6, 2.8 Hz, 1H), 3.51-3.45 (m, 4H), 1.57 (m, 2H), 1.34 (m, 4H), 1.24 (m, 9H), 1.15 (m, 3H), 0.81 (t, J=7.2 Hz, 3H). ¹³C NMR (75 MHz, CDCl₃): δ 167.7, 146.8, 141.6, 129.5, 127.5, 120.8, 77.4, 45.2, 43.4, 31.7, 29.2, 29.2, 25.9, 22.6, 22.5, 22.3, 14.0. HRMS (ESI) m/z: [M+H]⁺ Calcd for C₂₀H₃₁N₂O₆ 395.2182; Found 395.2177.

Synthesis of Benzoxazinone Derivatives B2a, B2b and B2c.

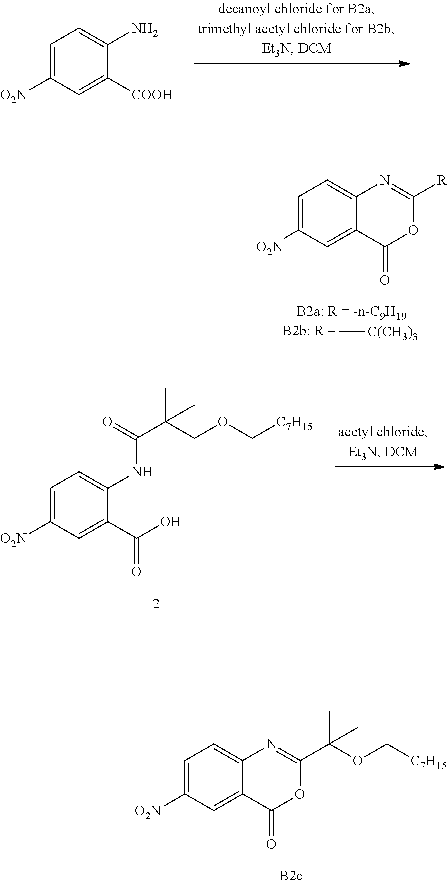

Compound B2a: To a solution of 2-amino-5-nitro benzoic acid (8.0 g, 30 mmol), triethylamine (7.8 g, 72 mmol) in CH₂Cl₂ was added decanoyl chloride (12.0 g, 63 mmol) dropwise. The reaction solution was then stirred at room temperature overnight. After filtering out any solid, the filtrate was concentrated and the residue was subject to silica gel column (hexane/CH₂Cl₂, from 2/1 to 1/1) to afford the pure benzoxazinone B2a as a white solid (6.39 g, 67%). ¹H NMR (400 MHz, CDCl₃): δ 9.05 (s, 1H), 8.61 (d, J=8.0 Hz, 1H), 7.73 (d, J=4.0 Hz, 1H), 2.74 (t, J=12.0 Hz, 2H), 1.85 (m, 2H), 1.36-1.29 (m, 12H), 0.89 (t, J=12.0 Hz, 3H). ¹³C NMR (75 MHz, CDCl₃): δ 116.6, 157.9, 150.7, 146.4, 130.5, 128.2, 124.4, 117.2, 34.9, 31.7, 29.3, 29.2, 29.1, 29.0, 25.9, 22.6, 14.0. MS (ESI) m/z: [M+H]⁺ Calcd. for C₁₇H₂₃N₂O₄ 319.2; Found 319.2.

Compound B2b: To a solution of 2-amino-5-nitro benzoic acid (8.0 g, 30 mmol) and triethylamine (7.8 g, 72 mmol) in dry CH₂Cl₂ (100 mL) was added trimethyl acetyl chloride (7.60 g, 63 mmol) dropwise. The resulting reaction solution was stirred at room temperature overnight. After filtering out any solid, the reaction solution was then concentrated and the residue was subject to silica gel column (hexane/CH₂Cl₂, from 2/1 to 1/1) to afford the pure benzoxazinone B2b as an off-white solid (5.88 g, 79%). ¹H NMR (400 MHz, CDCl₃): δ 9.05 (d, J=5.0 Hz, 1H), 8.61 (dd, J=10.0 and 5.0 Hz, 1H), 7.75 (d, J=10.0 Hz, 1H), 1.43 (s, 9H). ¹³C NMR (75 MHz, CDCl₃): δ 171.8, 158.2, 50.8, 146.5, 130.4, 128.5, 124.4, 117.2, 38.4, 27.6. MS (ESI) m/z: [M+H]⁺ Calcd. for C₁₂H₁₃N₂O₄ 249.1; Found 249.1.

Compound B2c: To a solution of acid 2 (19.7 g, 50 mmol) and triethyl amine (6.1 g, 60 mmol) in dry CH₂Cl₂ (250 mL) was added acetyl chloride (4.8 g, 60 mmol). The obtained reaction solution was stirred at room temperature overnight. After filtering out any solid, the reaction solution was concentrated and the residue was subject to silica gel column (hexane/CH₂Cl₂, from 2/1 to 1/1) to afford the pure benzoxazinone B2c as a yellowish oil (14.1 g, 75%). ¹H NMR (400 MHz, CDCl₃): δ 9.03 (d, J=5.0 Hz, 1H), 8.56 (dd, J=10.0 and 5.0 Hz, 1H), 7.75 (d, J=10.0 Hz, 1H), 3.59 (s, 2H), 3.40 (t, J=7.5 Hz, 2H), 1.46 (m, 2H), 1.39 (s, 6H), 1.16 (m, 10H), 0.83 (t, J=7.5 Hz, 3H). ¹³C NMR (75 MHz, CDCl₃): δ 169.9, 158.1, 150.7, 146.4, 130.3, 128.6, 124.4, 117.2, 77.2, 71.6, 43.3, 31.7, 29.3, 29.2, 26.0, 22.9, 14.0. MS (ESI) m/z: [M+H]⁺ Calcd. for C₂₀H₂₉N₂O₅ 377.2; Found 377.1.

Products of ring-opening of benzoxazinone derivatives with amines.

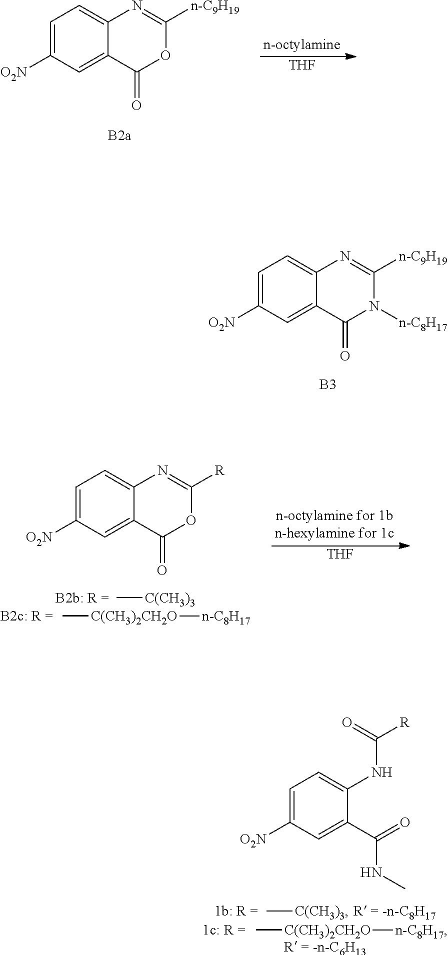

Compound B3: 1.59 g of benzoxazinone B2a (5.0 mmol) and n-octylamine (0.65 g, 5.0 mmol) was dissolved in dry THE (25 mL). The reaction solution was stirred at room temperature overnight. Upon the removal of THE under vacuum, the residue was subject to silica gel column (hexane/ethyl acetate, from 20/1 to 15/1) to afford the pure compound B3 as a yellowish solid (1.25 g, 58%). ¹H NMR (400 MHz, CDCl₃): δ 9.10 (d, J=4.0 Hz, 1H), 8.46 (dd, J=8.0, 4.0 Hz, 1H), 7.72 (d, J=8.0 Hz, 1H), 4.08 (t, J=12 Hz, 2H), 2.82 (t, J=12.0 Hz, 2H), 1.85 (m, 2H), 1.72 (m, 2H), 1.48-1.28 (m, 22H), 0.87 (m, 6H). ¹³C NMR (75 MHz, CDCl₃): δ 161.1, 160.8, 151.3, 145.1, 128.4, 127.9, 123.5, 120.3, 44.2, 35.2, 31.8, 31.7, 29.4, 29.3, 29.3, 29.2, 29.1, 28.8, 27.3, 26.9, 22.6, 22.6, 14.0, 14.0. HRMS (ESI) m/z: [M+H]⁺ Calcd. for C₂₅H₄₀N₃O₃ 430.3070; Found 430.3063.

Compound 1b: The compound 1b (3.76 g, 99%) as a white solid was prepared from benzoxazinone B2b (2.48 g, 10.0 mmol) and n-octylamine (1.29 g, 10.0 mmol) via the same method as the preparation of compound B3 without any modification. ¹H NMR (500 MHz, CDCl₃): δ 11.79 (s, 1H), 8.92 (d, J=10.0 Hz, 1H), 8.39 (s, 1H), 8.32 (dd, J=10.0 Hz, 1H), 6.33 (broad, 1H), 3.48 (dd, J=15.0 and 10.0 Hz, 2H), 1.67 (m, 2H), 1.39-1.29 (m, 20H), 0.89 (t, J=10.0 Hz, 3H). ¹³C NMR (75 MHz, CDCl₃): δ 178.4, 167.2, 145.8, 141.3, 127.4, 122.5, 121.0, 120.0, 40.4, 31.7, 29.3, 29.2, 29.1, 27.4, 26.9, 22.6, 14.0. HRMS (ESI) m/z: [M+Na]⁺ Calcd. for C₂₀H₃₁N₃NaO₄ 400.2212; Found 400.2197.

Compound 1c: The compound 1c (4.77 g, 99%) as a yellowish oil was prepared from benzoxazinone B2c (3.76 g, 10.0 mmol) and n-hexylamine (1.02 g, 10.0 mmol) via the same method as the preparation of compound B3 without any modification. ¹H NMR (400 MHz, CDCl₃): δ 11.62 (s, 1H), 8.95 (d, J=9.2 Hz, 1H), 8.36 (s, 1H), 8.28 (dd, J=9.2 Hz, 6.8 Hz, 1H), 6.36 (broad, 1H), 3.47-3.44 (m, 6H), 1.65 (m, 2H), 1.52 (m, 2H), 1.42-1.38 (m, 2H), 1.31 (m, 10H), 1.25-1.18 (m, 10H), 0.90 (t, J=6.8 Hz, 3H), 0.84 (t, J=7.2 Hz, 3H). ¹³C NMR (75 MHz, CDCl₃): δ 176.6, 167.0, 145.4, 141.3, 127.2, 122.5, 121.3, 120.8, 77.4, 71.7, 45.1, 40.4, 31.8, 31.4, 29.4, 29.3, 29.3, 29.3, 26.6, 26.0, 22.7, 22.6, 22.5, 14.0, 14.0. HRMS (ESI) m/z: [M+H]⁺ Calcd. for C₂₆H₄₄N₃O₅ 478.3281; Found 478.3272.

Synthesis of benzoxazinone dimer 2b′ and 2c′.

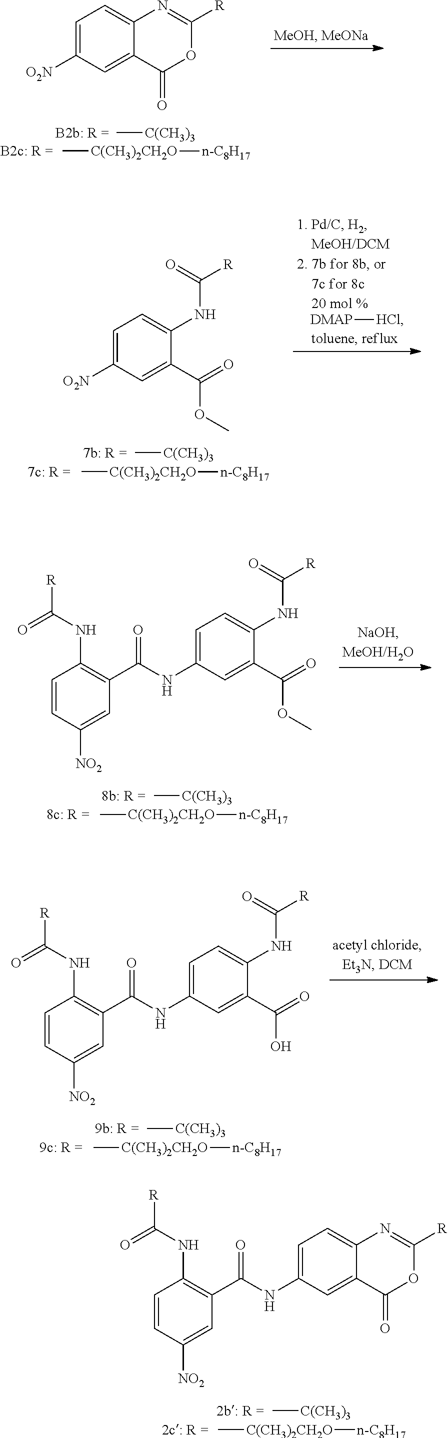

Compound 7b: To a solution of benzoxazinone B2b (4.96 g, 20.0 mmol) in methanol (100 mL) was added 10 mol % of sodium methoxide (0.11 g). The obtained reaction solution was stirred at room temperature overnight. Methanol was removed and the residue was then dissolved in ethyl acetate (100 mL). The organic layer was washed with brine (20 mL×2 times) and dried over anhydrous Na₂SO₄. The methyl ester 7b was obtained as an off-white solid after removing ethyl acetate (5.49 g, 98%). ¹H NMR (500 MHz, CDCl₃): δ 11.66 (s, 1H), 9.01 (d, J=10.0 Hz, 1H), 8.95 (s, 1H), 8.36 (d, J=10.0 Hz, 1H), 4.02 (s, 3H), 1.37 (s, 9H). ¹³C NMR (75 MHz, DMSO-d₆): δ 177.8, 167.0, 146.4, 141.6, 129.7, 126.6, 120.7, 116.3, 53.7, 27.4. HRMS (ESI) m/z: [M+Na]⁺ Calcd. for C₁₃H₁₆N₂NaO₅ 303.0957; Found 303.0952.

Compound 7c: The compound 7c (7.92 g, 97%) as a yellowish oil was prepared from the solution of benzoxazinone B2c (7.52 g, 20.0 mmol) and 10 mol % of sodium methoxide (0.11 g) in methanol (100 mL) via the same method as the preparation of compound 7b without any modification. ¹H NMR (400 MHz, CDCl₃): δ 11.60 (s, 1H), 9.01 (d, J=9.6 Hz, 1H), 8.93 (d, J=2.8 Hz, 1H), 8.36 (dd, J=9.6, 2.8 Hz, 1H), 3.99 (s, 3H), 3.47-3.44 (m, 4H), 1.51 (m, 2H), 1.33 (s, 6H), 1.21-1.17 (m, 10H), 0.83 (t, J=6.8 Hz, 3H). ¹³C NMR (75 MHz, CDCl₃): δ 176.6, 166.8, 146.7, 141.4, 129.1, 126.8, 120.6, 115.0, 77.3, 71.7, 52.9, 45.2, 31.7, 29.3, 29.3, 26.1, 22.7, 22.6, 22.4, 14.0. HRMS (ESI) m/z: [M+Na]⁺ Calcd. for C₂₁H₃₂N₂NaO₆ 431.2158; Found 431.2152.

Compound 8b: The reduction reaction of methyl ester 7b to prepare corresponding aromatic amine was accomplished via the general hydrogenation method. The freshly prepared aromatic amine (0.75 g, 3.0 mmol) and B2b (0.74 g, 3.0 mmol) were dissolved in dry toluene (50 mL) followed by addition of 20 mol % of DMAP HCl salt. The obtained suspended solution was heated under reflux overnight. The organic layer was then washed with brine (30 mL×2 times) and dried over anhydrous Na₂SO₄. After removal of toluene under vacuum, the obtained residue was dissolved in the hot methanol. The white precipitate then formed and was collected by filtration to give the pure compound 8b as an off-white solid (1.39 g, 93%). ¹H NMR (500 MHz, CDCl₃): δ 11.49 (s, 1H), 11.24 (s, 1H), 9.10 (s, 1H), 8.73 (d, J=9.5 Hz, 1H), 8.63 (d, J=9.5 Hz, 1H), 8.60 (d, J=2.5 Hz, 1H), 8.34 (d, J=2.5 Hz, 1H), 8.20 (dd, J=9.5, 2.5 Hz, 1H), 7.65 (dd, J=9.5, 2.5 Hz, 1H), 3.98 (s, 3H). 1.38 (s, 9H), 1.32 (s, 9H). ¹³C NMR (75 MHz, CDCl₃): δ177.5, 176.9, 168.0, 166.2, 145.4, 141.8, 137.9, 133.1, 127.9, 127.7, 125.1, 123.9, 121.5, 121.2, 121.0, 116.8, 53.1, 27.6, 27.4. HRMS (ESI) m/z: [M-MeOH+H]⁺ Calcd. for C₂₄H₂₇N₄O₆ 467.1931; Found 467.1921.

Compound 8c: The reduction reaction of methyl ester 7c to prepare corresponding aromatic amine was accomplished via the general hydrogenation method. To a solution of freshly prepared aromatic amine (1.13 g, 3.0 mmol) and B2c (1.13 g, 3.0 mmol) in dry toluene (50 mL) was added 20 mol % of DMAP HCl salt. The obtained suspended solution was heated under reflux overnight. The organic layer was then washed with brine (30 mL×2 times) and dried over anhydrous Na₂SO₄. After removal of toluene under vacuum, the residue was subject to flash silica gel column (hexane/ethyl acetate, from 5/1 to 2/1) to afford the pure compound 8c as a yellowish oil (2.01 g, 89%). ¹H NMR (400 MHz, CDCl₃): δ 11.35 (s, 1H), 11.21 (s, 1H), 8.92 (s, 1H), 8.73 (d, J=9.2 Hz, 1H), 8.64 (d, J=8.8 Hz, 1H), 8.58 (s, 1H), 8.34 (s, 1H), 8.20 (d, J=9.2 Hz, 1H), 7.66 (d, J=8.8 Hz, 1H), 3.95 (s, 3H), 3.51 (s, 2H), 3.45-3.39 (m, 6H), 1.54 (m, 2H), 1.45 (m, 2H), 1.35 (s, 6H), 1.28 (s, 6H), 1.22-1.12 (m, 20H), 0.84-0.81 (m, 6H). ¹³C NMR (75 MHz, CDCl₃): δ 176.9, 176.4, 167.7, 165.5, 144.8, 141.7, 138.1, 132.2, 127.2, 127.1, 123.6, 123.4, 122.0, 121.8, 121.3, 116.0, 77.5, 77.3, 71.8, 71.7, 52.4, 45.0, 44.9, 31.8, 31.7, 29.3, 29.2, 26.1, 26.0, 25.9, 22.8, 22.7, 22.6, 22.6, 22.4, 14.1, 14.0. HRMS (ESI) m/z: [M+H]⁺ Calcd. for C₄₁H₆₃N₄O₉ 755.4595; Found 755.4627.

Compound 9b: To a solution of methyl ester 8b (1.00 g, 2.0 mmol) in methanol (30 mL) was added an aqueous NaOH solution (0.32 g of NaOH in 1 mL of H₂O). The reaction solution was stirred at room temperature overnight. Then methanol was removed and the residue was acidified with diluted HCl aqueous solution. The resulting aqueous layer was then extracted with ethyl acetate (50 mL×2 times). The organic layer was then washed with brine and dried over anhydrous Na₂SO₄. The removal of ethyl acetate under vacuum afforded the acid 9b as an off-white solid (0.94 g, 97%). ¹H NMR (500 MHz, CDCl₃): δ 11.54 (s, 1H), 11.05 (s, 1H), 8.93 (d, J=11.5 Hz, 1H), 8.79 (d, J=11.5 Hz, 1H), 8.63 (s, 1H), 8.35-8.33 (m, 2H), 8.21 (s, 1H), 7.82 (d, J=11.5 Hz, 1H), 1.35 (s, 9H), 1.32 (s, 9H). ¹³C NMR (75 MHz, DMSO-d₆): δ 177.5, 176.9, 169.9, 166.1, 145.5, 141.7, 138.6, 132.9, 127.9, 127.6, 125.1, 124.4, 121.4, 120.9, 120.4, 116.7, 27.6, 27.4. HRMS (ESI) m/z: [M+Na]⁺ Calcd. for C₂₄H₂₈N₄NaO₇ 507.1856; Found 507.1845.

Compound 9c: The hydrolysis of methyl ester 8c (1.51 g, 2.0 mmol) to prepare acid 9c (1.39 g, 94%) was carried out using the same method as the preparation of compound 9b without any modification. ¹H NMR (400 MHz, CDCl₃): δ 11.58 (s, 1H), 10.82 (s, 1H), 9.24 (s, 1H), 8.89-8.86 (m, 2H), 8.43 (d, J=8.8 Hz, 1H), 8.32 (d, J=9.2 Hz, 1H), 7.87-7.85 (m, 2H), 3.57 (m, 2H), 3.45-3.39 (m, 6H), 1.52 (m, 2H), 1.45 (m, 2H), 1.36 (m, 6H), 1.29 (m, 6H), 1.23 (m, 4H), 1.15-1.11 (m, 16H), 0.81-0.79 (m, 6H). ¹³C NMR (75 MHz, CDCl₃): δ 176.8, 176.3, 165.7, 145.4, 141.7, 138.3, 132.6, 127.5, 124.3, 123.6, 121.9, 121.6, 121.0, 115.3, 77.8, 77.2, 72.0, 71.7, 45.1, 44.8, 31.7, 31.7, 29.3, 29.2, 26.0, 26.0, 23.0, 22.8, 22.6, 22.5, 14.0, 14.0. HRMS (ESI) m/z: [M+Na]⁺ Calcd. for C₄₀H₆₀N₄NaO₉ 763.4258; Found 763.4275.

Compound 2b′: To a solution of acid 9b (0.48 g, 1.0 mmol) and triethyl amine (0.25 g, 2.5 mmol) in dry DCM (10 mL) was added acetyl chloride (0.16 g, 2.0 mmol) dropwise. The reaction solution was stirred at room temperature overnight. Any solid was filtered out and the obtained solution was concentrated under high vacuum. The residue was then subject to flash silica gel column (CH₂Cl₂/ethyl acetate, from 10/1 to 5/1) to afford pure compound 2b′ as an off-white solid (0.33 g, 71%). ¹H NMR (500 MHz, CDCl₃): δ 11.51 (s, 1H), 8.99 (s, 1H), 8.92 (d, J=9.5 Hz, 1H), 8.71 (s, 1H), 8.45 (s, 1H), 8.34 (d, J=9.5 Hz, 1H), 8.28 (d, J=8.5 Hz, 1H), 7.68 (d, J=8.5 Hz, 1H), 1.38 (s, 9H), 1.34 (s, 9H). ¹³C NMR (75 MHz, DMSO-d₆): δ 177.5, 167.3, 166.4, 159.6, 145.4, 142.8, 141.8, 138.3, 129.9, 128.0, 127.7, 125.2, 121.6, 121.2, 119.2, 117.1, 37.9, 27.9, 27.4. MS (ESI) m/z: [M+H]⁺ Calcd. for C₂₄H₂₇N₄O₆ 467.2; Found 467.1.

Compound 2c′: To a solution of acid 9c (0.74 g, 1.0 mmol) and triethyl amine (0.25 g, 2.5 mmol) in dry DCM (10 mL) was added acetyl chloride (0.16 g, 2.0 mmol) dropwise. The reaction solution was stirred at room temperature overnight. Any solid was filtered out and the obtained solution was concentrated under high vacuum. The residue was then subject to flash silica gel column (hexane/ethyl acetate, from 5/1 to 2/1) to afford pure compound 2c′ as an off-white solid (0.48 g, 67%). ¹H NMR (400 MHz, CDCl₃): δ 11.28 (s, 1H), 8.85 (d, J=7.2 Hz, 1H), 8.79 (s, 1H), 8.64 (d, J=2.0 Hz, 1H), 8.43 (d, J=2.0 Hz, 1H), 8.32 (dd, J=7.2, 2.0 Hz, 1H), 8.30 (dd, J=6.8, 2.0 Hz, 1H), 7.66 (d, J=6.8 Hz, 1H), 3.58 (s, 2H), 3.46-3.41 (m, 6H), 1.49 (m, 4H), 1.37 (s, 6H), 1.30 (s, 6H), 1.23-1.13 (m, 20H), 0.84-0.82 (m, 6H). ¹³C NMR (75 MHz, CDCl₃): δ 176.7, 165.8, 165.4, 159.9, 145.3, 143.6, 141.6, 137.0, 129.0, 128.0, 127.8, 123.0, 122.0, 121.1, 118.9, 117.3, 77.2, 71.8, 71.7, 45.0, 42.8, 31.7, 29.3, 29.2, 26.0, 26.0, 23.0, 22.8, 22.6, 14.1. MS (ESI) m/z: [M+H]⁺ Calcd. for C₄₀H₅₉N₄O₈ 723.4; Found 723.1.

Stepwise chain elongation reaction via the DMAP HCl mediated ring opening of benzoxazinone.

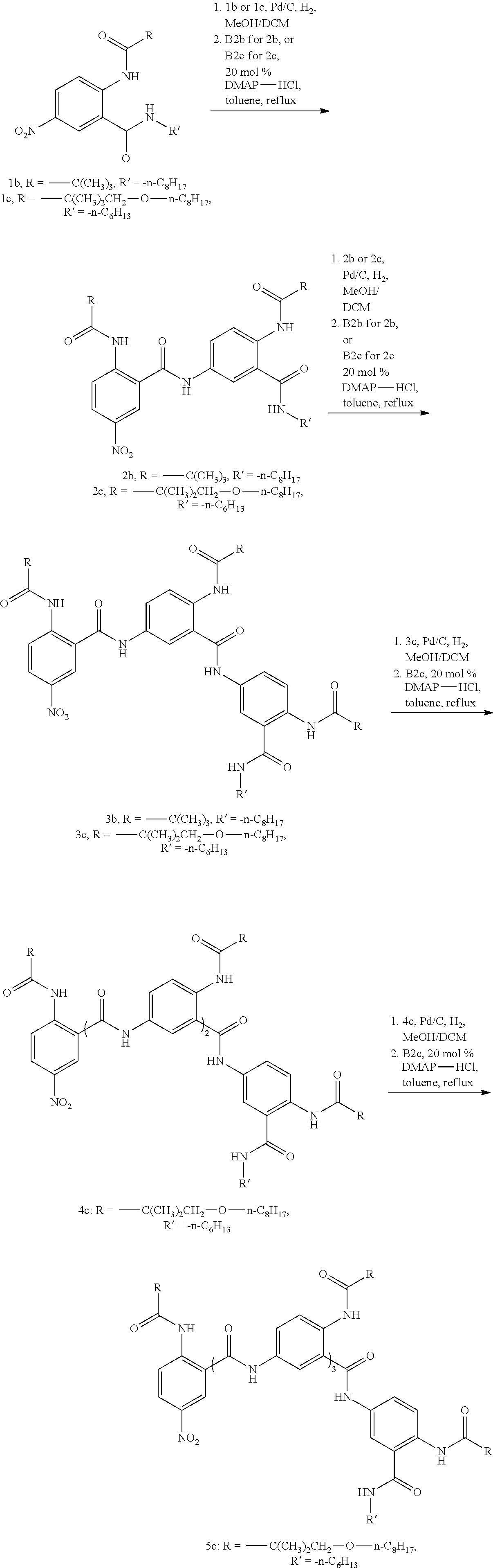

Compound 2b: The reduction of nitro 1b to its corresponding aromatic amine was accomplished via the general hydrogenation method. To a solution of freshly prepared aromatic amine (1.04 g, 3.0 mmol) and B2b (0.74 g, 3.0 mmol) in dry toluene (50 mL) was added 20 mol % of DMAP HCl salt. The obtained suspended solution was heated under reflux overnight with vigorously stirring. The organic layer was washed with brine (20 mL×2 times) and dried over anhydrous Na₂SO₄. After removal of toluene under vacuum, the obtained residue was dissolved in hot methanol. The white precipitate then formed and was collected by filtration to give pure compound 2b (1.75 g, 98%). ¹H NMR (500 MHz, CDCl₃): δ 11.50 (s, 1H), 11.10 (s, 1H), 9.16 (s, 1H), 8.78 (d, J=9.5 Hz, 1H), 8.64 (d, J=2.5 Hz, 1H), 8.44 (d, J=9.0 Hz, 1H), 8.23 (dd, J=9.5, 3.0 Hz, 1H), 7.99 (d, J=2.5 Hz, 1H), 7.35 (dd, J=9.5, 2.5 Hz, 1H), 6.43 (t, J=6.0 Hz, 1H), 3.48 (m, 2H), 1.65 (m, 2H), 1.42-1.28 (m, 28H), 0.88 (t, J=13.5 Hz, 3H). ¹³C NMR (75 MHz, CDCl₃): δ 178.7, 178.2, 168.4, 165.9, 145.4, 141.5, 136.3, 132.1, 127.4, 124.9, 123.6, 122.1, 122.0, 121.5, 120.7, 119.8, 40.5, 40.2, 40.1, 31.8, 29.4, 29.3, 29.2, 27.5, 27.3, 26.9, 22.6, 14.1. HRMS (ESI) m/z: [M+Na]⁺ Calcd. for C₃₂H₄₅N₅NaO₆ 618.3268; Found 618.3258.

Compound 2c: The reduction of nitro 1c to its corresponding aromatic amine was accomplished via the general hydrogenation method. The coupling reaction between freshly prepared aromatic amine (1.34 g, 3.0 mmol) and B2c (1.13 g, 3.0 mmol) was carried out in the same method as the preparation of compound 2b. After removal of toluene, the obtained residue was subject to flash silica gel column (hexanes/ethyl acetate, from 5/1 to 2/1) to afford the pure compound 2c (2.35 g, 95%) as a yellowish oil. ¹H NMR (400 MHz, CDCl₃): δ 11.42 (s, 1H), 11.07 (s, 1H), 9.10 (s, 1H), 8.80 (d, J=9.2 Hz, 1H), 8.69 (d, J=2.8 Hz, 1H), 8.33 (d, J=9.2 Hz, 1H), 8.26 (dd, J=9.2, 2.8 Hz, 1H), 7.84 (d, J=2.4 Hz, 1H), 7.35 (dd, J=9.2, 2.4 Hz, 1H), 6.55 (t, J=6.0 Hz, 1H), 3.50 (s, 2H), 3.44 (s, 2H), 3.42 (m, 4H), 3.38 (m, 2H), 1.65 (m, 2H), 1.53 (m, 2H), 1.42 (m, 6H), 1.31 (m, 10H), 1.28 (m, 8H), 1.20 (m, 10H), 1.12 (m, 6H), 0.89-0.80 (m, 9H). ¹³C NMR (75 MHz, CDCl₃): δ 176.8, 176.2, 168.2, 165.6, 145.1, 141.6, 135.9, 132.2, 127.3, 125.0, 123.4, 122.4, 122.1, 121.7, 121.4, 119.9, 77.6, 77.3, 71.8, 71.7, 45.0, 44.6, 40.1, 31.8, 31.7, 31.5, 29.4, 29.4, 29.3, 29.2, 29.2, 26.7, 26.0, 26.0, 22.9, 22.7, 22.6, 22.6, 22.6, 14.1, 14.0. HRMS (ESI) m/z: [M+Na]⁺ Calcd. for C₄₆H₇₃N₅NaO₈ 846.5357; Found 846.5340.

Compound 3b: Reduction of nitro 2b to its corresponding amine was accomplished via the general hydrogenation method. To a solution of freshly prepared amine (1.70 g, 3.0 mmol) and benzoxazinone B2b (0.74 g, 3.0 mmol) in dry toluene (100 mL) was added 20 mol % of DMAP HCl salt. The obtained suspended solution was heated under reflux overnight with vigorous stirring. The organic layer was then washed with brine (20 mL×2 times) and dried over anhydrous Na₂SO₄. After removal of toluene, the obtained crude product was washed with hot ethyl acetate to afford the compound 3b (2.31 g, 95%) as a white solid. ¹H NMR (500 MHz, CDCl₃): δ 11.50 (s, 1H), 11.10 (s, 1H), 9.16 (s, 1H), 8.77 (d, J=10.0 Hz, 1H), 8.64 (s, 1H), 8.43 (d, J=10.0 Hz, 1H), 8.25 (d, J=10.0 Hz, 1H), 7.99 (s, 1H), 7.35 (d, J=5.0 Hz, 1H), 6.43 (t, J=7.5 Hz, 1H), 3.48 (m, 2H), 1.66 (m, 2H) 1.42-1.35 (m, 28H), 0.88 (t, J=7.5 Hz, 3H). ¹³C NMR (75 MHz, CDCl₃): δ 178.7, 178.2, 168.4, 165.9, 145.4, 141.5, 136.3, 132.1, 127.4, 124.9. 123.6, 122.1, 122.0, 121.5, 120.7, 119.8, 40.5, 40.2, 40.1, 31.8, 29.4, 29.3, 29.2, 27.5, 27.3, 26.9, 22.6, 14.1. HRMS (ESI) m/z: [M+Na]⁺ Calcd. for C₄₄H₅₉N₇NaO₈ 836.4323; Found 836.4306.

Compound 3c: The reduction reaction of nitro 2c to its corresponding amine was accomplished via the general hydrogenation method. The coupling reaction between freshly prepared amine (2.38 g, 3.0 mmol) and benzoxazinone B2c (1.13 g, 3.0 mmol) was carried out in the same method as the preparation of compound 3b. After removal of toluene, the obtained residue was then dissolved in the hot acetonitrile. The white solid then formed and was collected by filtration to afford the pure compound 3c as a white solid (3.23 g, 92%). ¹H NMR (400 MHz, 10% DMSO-d₆/90% CDCl₃): δ 11.64 (s, 1H), 11.14 (s, 1H), 10.73 (s, 1H), 10.59 (s, 1H), 9.84 (s, 1H), 8.90 (d, J=9.2 Hz, 1H), 8.85 (d, J=2.4 Hz, 1H), 8.58-8.52 (m, 2H), 8.33 (dd, J=9.6, 2.8 Hz, 1H), 8.11 (m, 2H), 7.93 (dd, J=9.2, 2.4 Hz, 1H), 7.74 (dd, J=9.2, 2.4 Hz, 1H), 7.20 (t, J=5.6 Hz, 1H), 3.48-3.42 (m, 14H), 1.62 (m, 2H), 1.55-1.47 (m, 6H), 1.39 (m, 2H), 1.30-1.28 (m, 26H), 1.23 (m, 10H), 1.19 (m, 10H), 1.15 (m, 6H), 0.89-0.83 (m, 12H). ¹³C NMR (75 MHz, 10% DMSO-d₆/90% CDCl₃): δ 176.3, 175.5, 175.4, 168.4, 166.9, 165.7, 145.5, 141.2, 135.5, 135.4, 133.2, 133.0, 127.1, 124.7, 124.2, 123.7, 123.4, 122.2, 122.1, 121.7, 121.0, 120.8, 119.1, 77.5, 77.1, 77.0, 71.5, 44.9, 44.4, 44.3, 31.6, 31.6, 31.5, 31.3, 29.3, 29.2, 29.1, 29.1, 29.1, 29.0, 26.5, 25.9, 25.8, 22.8, 22.7, 22.7, 22.4, 22.4, 14.0, 13.9. HRMS (ESI) m/z: [M+H]⁺ Calcd. for C₆₆H₁₀₄N₇O₁₁ 1170.7794; Found 1170.7795.

Compound 4c: Reduction of nitro 3c to its corresponding amine was accomplished via the general hydrogenation method. To a solution of freshly prepared amine (3.42 g, 3.00 mmol) and benzoxazinone B2c (1.13 g, 3.00 mmol) in dry toluene (100 mL) was added 20 mol % of DMAP HCl salt. The obtained suspended solution was heated under reflux for 2 days with vigorous stirring. Upon the complete consumption of starting materials, the organic layer was then washed with brine (20 mL×2 times) and dried over anhydrous Na₂SO₄. After removal of toluene under vacuum, the obtained residue was then dissolved in hot acetonitrile. The white solid formed and was then collected by filtration to afford the pure compound 4c as a white solid (4.23 g, 93%). ¹H NMR (400 MHz, 10% DMSO-d₆/90% CDCl₃): δ 11.60 (s, 1H), 11.15 (s, 1H), 10.72 (m, 2H), 10.60 (s, 1H), 9.96 (s, 1H), 9.77 (s, 1H), 8.91 (d, J=9.6 Hz, 1H), 8.84 (d, J=2.8 Hz, 1H), 8.57-8.49 (m, 3H), 8.33 (dd, J=9.6, 2.8 Hz, 1H), 8.19 (s, 1H), 8.14 (d, J=2.4 Hz, 1H), 8.09 (s, 1H), 7.91 (dd, J=8.8, 2.4 Hz, 1H), 7.82 (dd, J=8.8, 2.4 Hz, 1H), 7.73 (dd, J=8.8, 2.4 Hz, 1H), 7.22 (t, J=6.0 Hz, 1H), 3.48-3.41 (m, 18H), 1.61 (m, 2H), 1.50 (m, 8H), 1.29-1.19 (m, 70H), 0.89-0.83 (m, 15H). ¹³C NMR (75 MHz, 10% DMSO-d₆/90% CDCl₃): δ 176.2, 175.5, 175.4, 168.4, 167.0, 166.9, 165.8, 145.4, 141.2, 135.6, 135.4, 135.1, 133.4, 133.1, 133.0, 128.8, 128.0, 127.1, 124.8, 124.2, 124.1, 123.8, 123.5, 123.4, 122.4, 122.0, 121.7, 121.0, 120.8, 120.0, 119.1, 77.5, 77.1, 77.0, 71.5, 44.9, 44.4, 44.3, 31.6, 31.3, 29.3, 29.2, 29.1, 29.1, 29.0, 26.5, 25.9, 25.8, 22.8, 22.7, 22.6, 22.5, 22.4, 14.0, 13.9. HRMS (ESI) m/z: [M+Na]⁺ Calcd. for C₈₆H₁₃₃N₉NaO₁₄ 1538.9870; Found 1538.9953.

Compound 5c: Reduction of nitro 4c to its corresponding amine was accomplished via the general hydrogenation method. To a solution of freshly prepared amine (4.46 g, 3.00 mmol) and benzoxazinone B2c (1.13 g, 3.00 mmol) in dry toluene (100 mL) was added 20 mol % of DMAP HCl salt. The obtained suspended solution was heated under reflux for 3 days with vigorous stirring. The organic layer was then washed with brine (20 mL×2 times) and dried over anhydrous Na₂SO₄. After removal of toluene under vacuum, the obtained residue was dissolved in hot acetonitrile. The white solid then formed and was collected by filtration to afford pure compound 5c as a white solid (5.08 g, 91%). ¹H NMR (400 MHz, 10% DMSO-d₆/90% CDCl₃): δ 11.57 (s, 1H), 11.15 (s, 1H), 10.72 (s, 1H), 10.70 (s, 1H), 10.66 (s, 1H), 10.61 (s, 1H), 9.99 (s, 1H), 9.91 (s, 1H), 9.78 (s, 1H), 8.90 (d, J=9.6 Hz, 1H), 8.84 (d, J=2.4 Hz, 1H), 8.57 (d, J=8.8 Hz, 1H), 8.52-8.49 (m, 3H), 8.33 (d, J=8.4 Hz, 1H), 8.25 (s, 1H), 8.19 (s, 1H), 8.15 (s, 1H), 8.10 (s, 1H), 7.90 (d, J=9.2 Hz, 1H), 7.80-7.75 (m, 2H), 7.72 (d, J=10.0 Hz, 1H), 7.19 (t, J=6.8 Hz, 1H), 3.48-3.40 (m, 22H), 1.60 (m, 2H), 1.52-1.45 (m, 10H), 1.29-1.27 (m, 42H), 1.23-1.19 (m, 44H), 0.88-0.83 (m, 18H). ¹³C NMR (75 MHz, 10% DMSO-d₆/90% CDCl₃): δ 176.2, 175.5, 175.3, 168.4, 167.1, 167.0, 166.9, 165.8, 145.4, 141.2, 135.6, 135.4, 135.1, 133.4, 133.4, 133.1, 133.0, 127.2, 124.7, 124.2, 124.1, 123.8, 123.5, 123.5, 122.4, 122.0, 121.6, 121.6, 121.0, 121.0, 120.8, 120.0, 119.1, 77.5, 71.5, 44.3, 31.6, 31.3, 29.3, 29.1, 29.0, 26.5, 25.9, 25.8, 22.8, 22.7, 22.6, 22.4, 22.4, 14.0. HRMS (MALDI) m/z: [M+Na]*Calcd. for C₁₀₆H₁₆₃N₁₁NaO₁₇ 1886.2160; Found 1886.2136.

[4+2] chain elongation reaction via the DMAP HCl mediated ring opening of benzoxazinone.

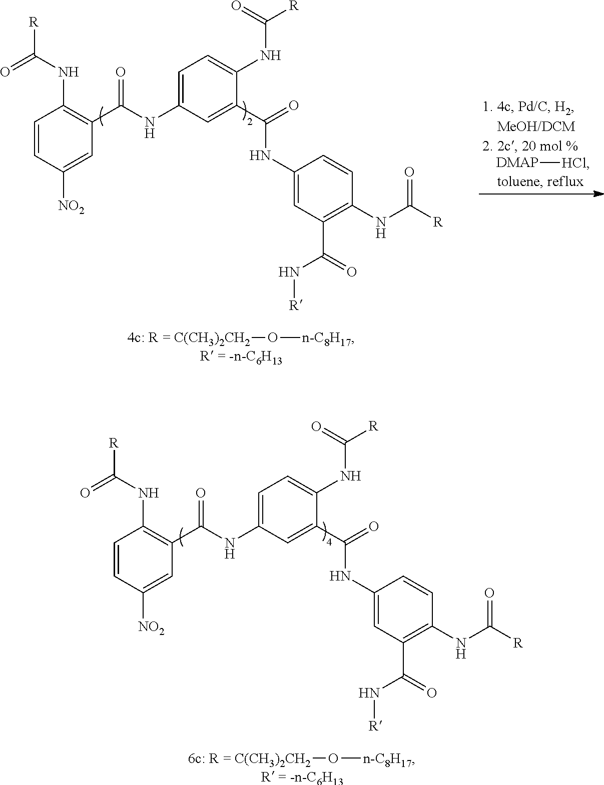

Compound 6c: The reduction reaction of nitro 4c to prepare its corresponding aromatic amine was accomplished via the general hydrogenation method. To a solution of freshly prepared aromatic amine (2.23 g, 1.50 mmol) and benzoxazinone dimer 2c′ (1.09 g, 1.50 mmol) in dry toluene (100 mL) was added 20 mol % of DMAP HCl salt. The obtained suspended solution was heated under reflux for 3 days. The organic layer was then washed with brine and dried over anhydrous Na₂SO₄. After removal of toluene under vacuum, the residue was dissolved in hot acetonitrile. The white precipitate then formed and was collected by filtration to give pure compound 6c as an off-white solid (3.05 g, 92%). ¹H NMR (400 MHz, 10% DMSO-d₆/90% CDCl₃): δ 11.56 (s, 1H), 11.15 (s, 1H), 10.73 (s, 1H), 10.70 (s, 1H), 10.64 (m, 2H), 10.62 (s, 1H), 10.02 (s, 1H), 9.96 (m, 2H), 9.80 (s, 1H), 8.89 (d, J=9.2 Hz, 1H), 8.83 (d, J=2.8 Hz, 1H), 8.57 (d, J=9.2 Hz, 1H), 8.51 (m, 4H), 8.33 (d, J=8.0 Hz, 1H), 8.26 (m, 2H), 8.22 (s, 1H), 8.15 (s, 1H), 8.11 (s, 1H), 7.89 (d, J=8.8 Hz, 1H), 7.77-7.69 (m, 4H), 7.23 (t, J=7.2 Hz, 1H), 3.47-3.45 (m, 14H), 3.42-3.40 (m, 12H), 1.61 (m, 2H), 1.49 (m, 14H), 1.29-1.26 (m, 52H), 1.23 (m, 12H), 1.18-1.15 (m, 36H), 0.85-0.82 (m, 21H). ¹³C NMR (75 MHz, 10% DMSO-d₆/90% CDCl₃): δ 176.2, 175.5, 175.4, 168.4, 167.1, 167.1, 167.0, 165.8, 145.4, 141.3, 135.5, 135.3, 135.1, 133.5, 133.4, 133.2, 133.0, 127.2, 124.2, 124.1, 123.8, 123.6, 123.4, 122.4, 122.3, 122.0, 121.6, 121.0, 120.0, 120.0, 119.1, 77.5, 77.2, 71.5, 44.9, 44.4, 44.3, 31.6, 31.3, 29.2, 29.1, 29.0, 26.5, 25.8, 22.8, 22.6, 22.4, 14.0. HRMS (MALDI) m/z: [M+Na]⁺ Calcd. for C₁₂₆H₁₉₃N₁₃NaO₂₀ 2232.4416; Found 2232.4634.

X-ray Crystallography Data

Single crystals of all the compounds were obtained from hexane/ethyl acetate (1/2, v/v) by slow evaporation of solvents at room temperature. Single crystal data was collected by the X scan technique at 293 K for B2b and 100 K for 2b′ on an Agilent Super Nova Dual diffractometer with an Atlas S2 detector, using Moka (k=0.71073 Å). Data of the single crystal structure were solved by direct methods which revealed the positions of all non-hydrogen atoms, and were refined on F² by a full-matrix least squares procedure using anisotropic displacement parameters. All hydrogen atoms were placed in ideal geometry and were refined using a riding model.

TABLE 2
Crystal data and structure refinement for B2b and 2b′. Computer programs:
CrysAlisPro 1.171.38.43f (Rigaku OD, 2015), SHELXTL (Sheldrick, 2015)

| Identification code | B2b | 2b′ |
|---|---|---|
| Empirical formula | C₁₂ H₁₂ N₂ O₄ | C₂₄ H₂₆ N₄ O₆ |
| Formula weight | 248.24 | 466.49 |
| Temperature(K) | 293(2) K | 293(2) |
| Wavelength (Å) | 0.71073 | 0.71073 |
| Crystal system, space | Monoclinic, P2(1)/c | Triclinic, P-1 |
| group | | |
| a, b, c (Å) | 5.9968(14), 23.292(4), | 11.4728(5), 12.3051(6), |
| | 8.5071(14) | 17.1650(8) |
| α, β, γ (°) | 90, 99.92(2), 90 | 80.909(4), 71.174(4), |
| | | 86.760(4) |
| Volume (Å³) | 1170.5(4) | 2264.76(18) |
| Z, Dₓ (Mg · m⁻³) | 4, 1.409 | 4, 1.368 |
| μ (mm⁻¹) | 0.108 | 0.100 |
| F(000) | 520 | 984 |
| Crystal size (mm) | 0.150 × 0.130 × 0.110 | 0.15 × 0.13 × 0.12 |
| Theta range for data | 2.58 to 23.98 | 1.90 to 25.00 |
| collection(deg.) | | |
| Limiting indices | −6 <= h <= 6, −26 <= k <= 20, | −13 ≤ h ≤ 13, −14 ≤ k ≤ 14, |
| | −9 <= 1 <= 9 | −18 ≤ 1 ≤ 20 |
| Reflections collected/ | 5480/1807 | 15168/7988 |
| unique | [R(int) = 0.0404] | [R(int) = 0.0247] |
| Completeness to | 99.0% | 100.0% |
| theta = 68.17 | | |
| Refinement | Full-matrix least-squares | Full-matrix least-squares |
| method | on F² | on F\hat{ }2 |
| Data/restraints/ | 1807/6/152 | 7988/0/613 |
| parameters | | |
| Goodness-of-fit on F² | 1.122 | 0.928 |
| Final R indices | R₁ = 0.0825, wR₂ = 0.2139 | R₁ = 0.0429, wR₂ = 0.0989 |
| [I > 2σ(I)] | | |
| R indices (all data) | R₁ = 0.1064, wR₂ = 0.2299 | R₁ = 0.0554, wR₂ = 0.1068 |
| Δρₘₐₓ, Δρₘᵢₙ (e Å⁻³) | 0.540 and −0.376 | 0.223 and −0.234 |

### Example 2

This example provides a description of anion receptors based on linear and cyclic aromatic oligoamides.

Design of Non-Cyclic, Anion-Binding Aromatic Oligoamides

Foldamers having inner cavities that are electrostatically positive, i.e., those with positive charges or multiple 1H-bond donors, are very rare. The availability of foldamers with such cavities will address current challenges in the binding, recognition, or transport of species especially various anions.

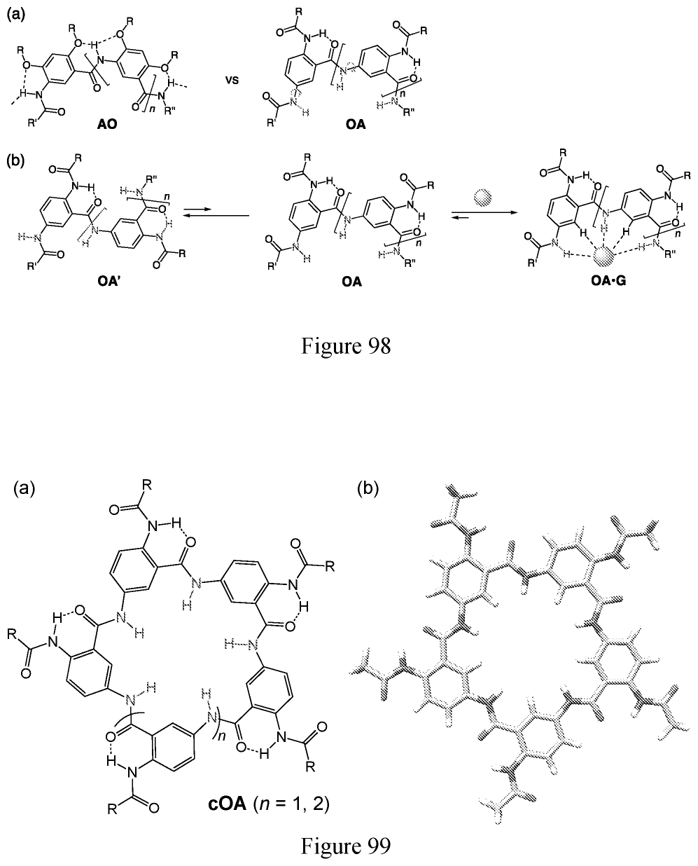

As shown in FIG. 98a, inverting the orientation of the backbone amide groups of oligoamide AO leads to oligomer OA. To ensure the inversion of each backbone amide group, a highly favorable, six-membered (S(6)-type) intramolecular H-bond is introduced between the oxygen atom of each backbone amide group and the amide proton of an adjacent acylamino side chain. Such an intramolecular H-bond keeps the backbone oxygen atom of the backbone amide group from engaging in additional, effective H-bonding and, at the same time, allows each backbone NH group to participate in intermolecular H-bonding interaction with anionic or polar molecular guests.

Unlike that of 1, the backbone of oligoamide OA is only partially constrained. Around each backbone amide group of OA, the rotation of the aryl-CO single bond is limited while the rotation of the C(O)NH-aryl bond remains relatively unhindered. It is thus expected that, in solution, oligoamide OA alone will adopt multiple conformations, each of which, such as OA′, exits in a small proportion (FIG. 98b). Among all the possibilities, conformation OA has all the amide protons being placed convergently. Upon adding a guest that undergoes H-bonding interactions with the backbone amide and aromatic protons, conformation OA is stabilized and the equilibrium is shifted toward complex OA·G, resulting in anion-induced folding of oligomer OA.

Design of cyclic, anion-binding aromatic oligoamides. Oligomer OA may also be cyclized into the corresponding macrocycle cOA in which the backbone amide NH groups are convergently placed, i.e., being forced to point toward the center of the macrocyclic structure (FIG. 99). Simple computer modeling indicates, due to the bond length and especially bond angles associated with the aromatic rings and the amide groups, macrocycles cOA comprising five or six residues i.e., being cyclic pentamer (5mer) or hexamer (6mer), are mostly likely to be obtained. Results from initial experimental studies (see below) indicate that the pentameric cOA macrocycles can be prepared in high yields. The macrocycles based on cOA are expected to exhibit significantly enhanced binding affinities toward anions since the cyclic structure serves to drastically reduce or even completely remove the entropy cost of organizing multiple NH groups.

Synthesis.

Synthesis of linear oligoamides: A new synthetic pathway based on a novel, highly efficient amide coupling method.

The synthesis of OA, oligomer of 5-amino-N-acylanthranlic acid, could not be performed based on established amide-coupling chemistry because anthranilic acid or its N-acylated derivative are known to undergo the self-cyclization when being subjected to acylating reagents such as acid chlorides or other amide-coupling agents. Specifically, the intramolecular cyclization involves the carboxyl group and the adjacent amino or acylamino group, giving derivatives having a 4H-3, 1-benzoxazin-4-one (or benzoxazinone) core. Such self-cyclization prevents the intended amide coupling reaction with other amines from happening. Indeed, an attempted coupling of acid A1 (Scheme 1) and an amino building block failed to yield any amide product.

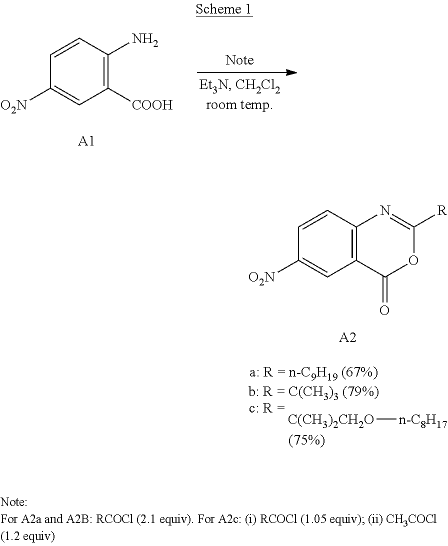

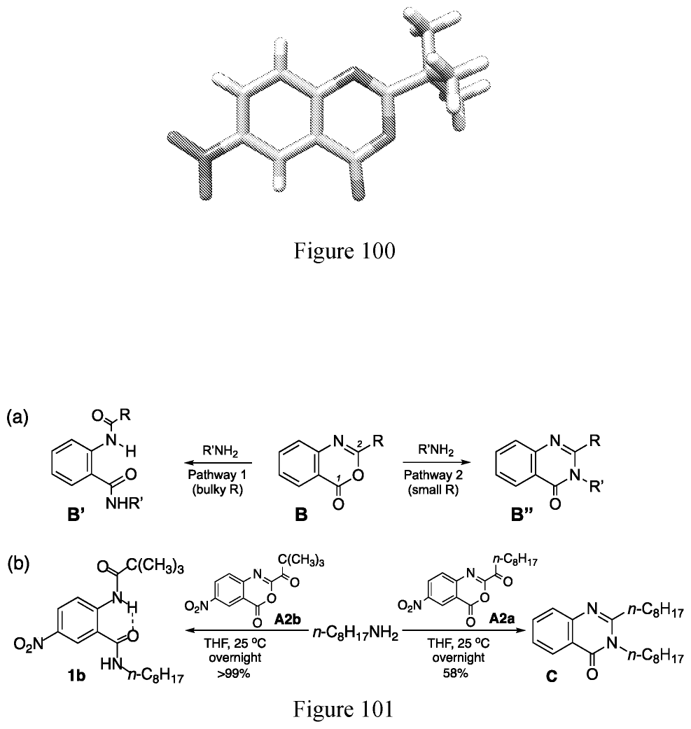

Treating 5-nitro-anthranlic acid A1 with two or more equivalents of decanoyl chloride or trimethylacetyl chloride gives benzoxazinone derivatives A2a or A2b in good yields (Scheme 1). Similarly, compound A2c, which differs with A2b in its side chain, was prepared by converting A1 into the corresponding 5-nitro-N-acylanthranlic acid that was then further treated with acetyl chloride to furnish the heterocycle. The ¹H NMR spectra of A2a, A2b, and A2c indicate that the three compounds share the same benzoxazinone core. The identities of A2a, and A2b, and A2c were further verified by ESI-MS spectra which confirm the corresponding molecular weights of the expected products. In addition, single crystals of A2b were obtained, allowing the determination of its X-ray structure (FIG. 100), which confirms that acid A1 indeed undergoes cyclization to give the benzoxazinone derivative as expected.

The reactions of (4H)-3, 1-benzoxazin-4-one B with amines were reported to follow one of two possible pathways (FIG. 101a). One involves the nucleophilic addition to carbonyl carbon C-1, leading to ring-opening product B′ with the release of an acylamino side chain and the formation of a new amide bond; the other gives quinazolone B″ upon nucleophilic addition to carbon C-2.

Steric factors play an important role in determining which one of the two pathways a reaction follows. When R and/or R′ are bulky and impose steric hindrance, ring-opening of B leads to the acylation of amine R′NH₂, which releases the acyamino (RCONH—) side chain and forms a new amide bond, thus resulting in the desired product B′; when R and/or R′ are small or linear groups that do not impose significant steric hindrance, heterocycle B″ is generated.

Compound A2a or A2b was treated with one equivalent of octylamine to assess the outcome of nucleophilic addition reactions involving these two benzoxazinone derivatives (FIG. 101b). It was observed that the reaction involving A2a afforded quinazolinone C in a yield of 58%, while the reaction of A2b and octylamine proceeded nearly quantitatively to give the ring-opening product, amide 1b. The molecular weights of A3 and 1b were confirmed by mass spectra. ¹H NMR spectra revealed two amide proton signals at 6.30 and 11.70 ppm for 1b while no amide signal can be detected for C. These results demonstrate that, to ensure the formation of the amide bond and the release of the acylamino side chain, an R group with a bulky a-carbon such as the t-butyl group of A2b need to be present.

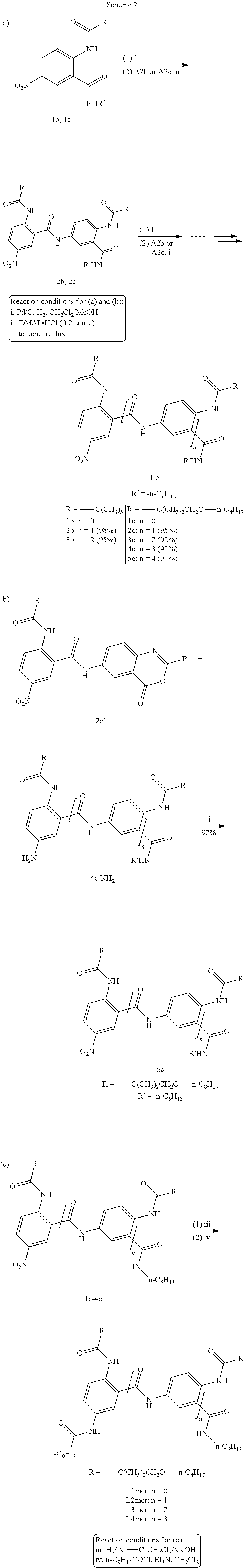

Based on the observation made with the reaction of A2b and n-octylamine, the synthesis of oligoamides represented by general structure OA (Scheme 2) was first probed by refluxing A2b and the amine derived from 1b (one equiv.) in toluene in the presence of 4-dimethylaminopyridine hydrochloride (DMAP, 0.2 equiv), which gave dimer 2b in 98% yield. Reducing 2b to the corresponding amine followed by coupling with A2b under the same condition produced trimer 3b again in very high (95%) yield. Attempts to prepare the tetramer bearing the same side chains of 1b-3b were hampered by the formation of an insoluble product that prevents characterization. Oligoamides 2c-5c, comprising two to five residues that bear side chains with a-quaternary carbons and n-octyloxy tails, are expected to show enhanced solubility. Indeed, starting from 1c by repetitive coupling of monomer A2c based on the step-wise steps and conditions for preparing oligomers 2b and 3b, oligoamides 2c-5c were obtained in very high yields of 95%, 92%, 93%, and 91%.

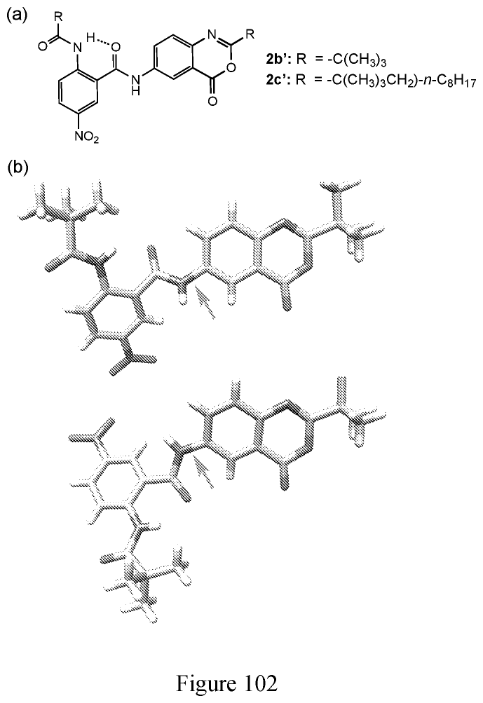

Dimeric 2b′ and 2c′ were prepared. ¹H NMR spectra demonstrate that 2b′ and 2c′ share the same backbone. The presence of the benzoxazinone unit is confirmed by the solid-sate structure of 2b′ (FIG. 102), in which two conformations related by ˜180° rotation around the single bond between the benzoxazinone unit and the rest of the molecule are revealed.

Under the same conditions for preparing oligoamides 2-5 based on the repetitive coupling of A2b or A2c, refluxing 2c′ and tetramer amine 4c-NH₂ in toluene gave hexamer 6c in 92% yield (Scheme 2b).

The highly efficient formation of oligoamides 2b, 3b, 2c-5c, and 6c indicates that the amide coupling steps adopted in this work, which is based on the ring-opening of benzoxazinone moiety, involves repetitive coupling of benzoxazinone monomers B2b and B2c to a growing oligomer chain, or the coupling of two oligomeric reactants like 2c′ and 4c-NH₂. This new and highly efficient synthetic method, which involves simple refluxing without the need of adding any coupling reagents, allows the preparation of aromatic oligoamides bearing acyamino side chains. Due to the self-cyclization of anthranlic acid and its N-acyl derivatives, aromatic oligoamides presented here cannot be prepared based on standard amide coupling chemistry and remained unknown until this work.

To ensure the formation of amide bonds, side chains having a bulky (quaternary) a-carbon as shown by A2b and A2c, and the corresponding oligoamides, are required. In addition to the bulky a-carbon, a wide variety of “tails” including alkyl, oligoether, and aliphatic chains bearing various solubilizing terminal groups such as amino, hydroxyl, and carboxyl groups, that are not limited to those of A2b and A2c, can be introduced to tune the solubility of the corresponding oligoamides.

Oligoamides L1mer, L2mer, L3mer, and L4mer were obtained by first reducing the nitro groups of 1c-4c followed by acylation with decanoyl chloride (Scheme 2c). With two, three, four, and five backbone NH groups, oligoamides L1mer-L4mer exhibit very good solubility in organic solvents such as chloroform and DMSO, which facilitates the study of their anion-binding behavior.

Synthesis of Cyclic Oligoamides Based on One-Pot Macrocyclization

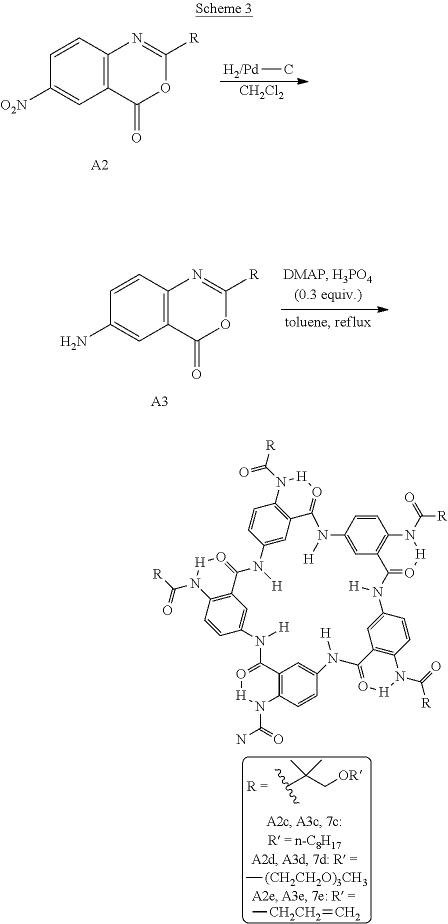

A macrocyclization procedure leading to the one-pot formation of pentameric macrocycles 7 has been discovered. This one-pot macrocyclization involves the self-condensation of monomer A3, prepared by reducing A2 with catalytic hydrogenation, by refluxing in toluene in the presence of DMAP and 0.3 equiv. of phosphoric acid. Macrocycles 7c-e have been obtained in good (60-70%) yields. These macrocycles exhibit good solubility in a variety of nonpolar and polar organic solvents. Besides, macrocycle 7d has a solubility in water in the millimolar concentration range.

Similar to the above discussion on side chains of the linear oligoamides, to ensure the formation of amide bonds and thus the success of the one-pot cyclization, side chains having a bulky (quaternary) a-carbon as shown by A2b and A2c, should be incorporated into the corresponding oligoamide macrocycles. In addition to the bulky a-carbon, a wide variety of “tails” including alkyl, oligoether, and aliphatic chains bearing various solubilizing terminal groups including, amino, hydroxyl, and carboxyl groups, that are not limited to those of A2b and A2c, can be introduced to tune the solubility of the corresponding oligoamide macrocycles.

Anion Binding.

Anion binding with linear oligoamides: Systematically tunable binding strength.

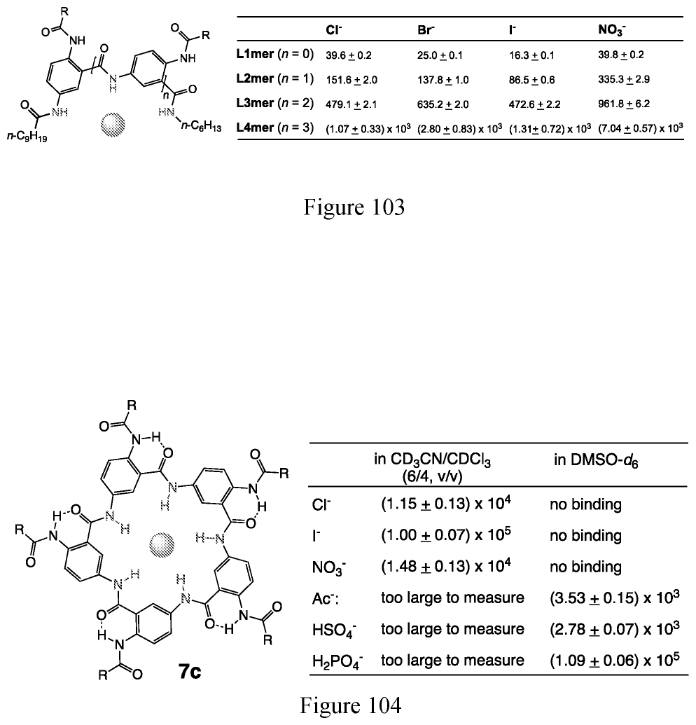

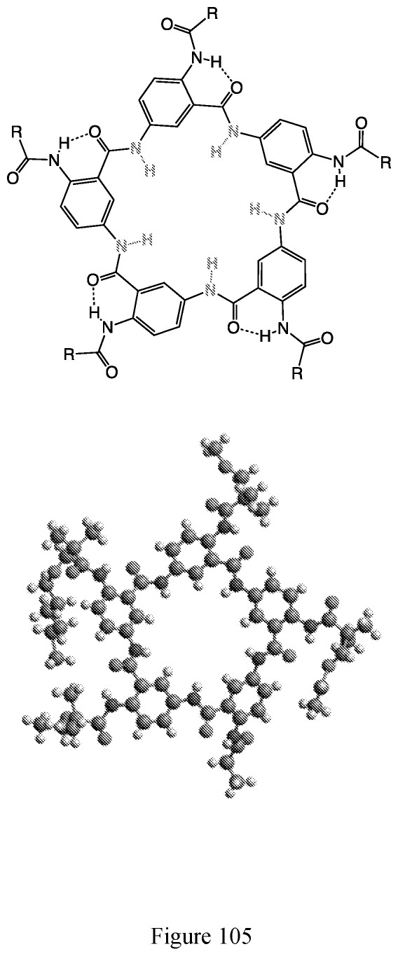

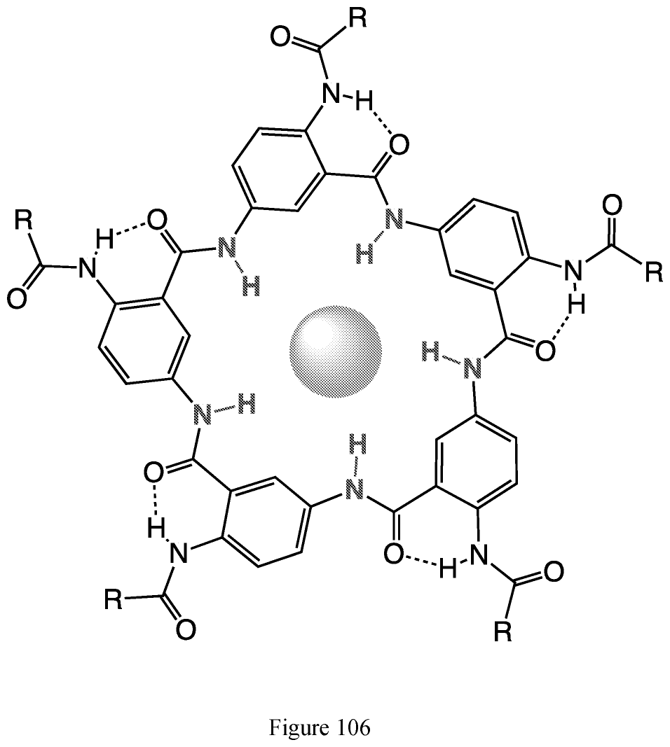

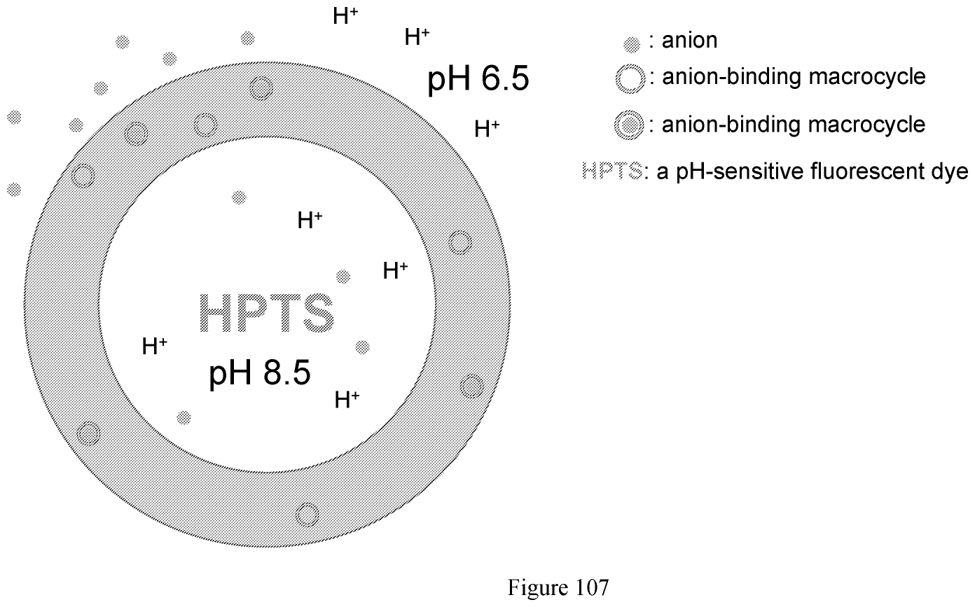

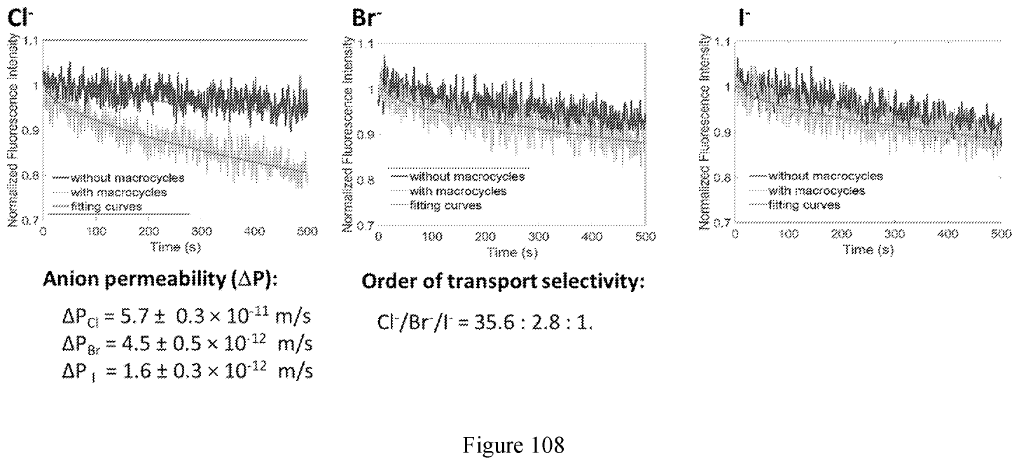

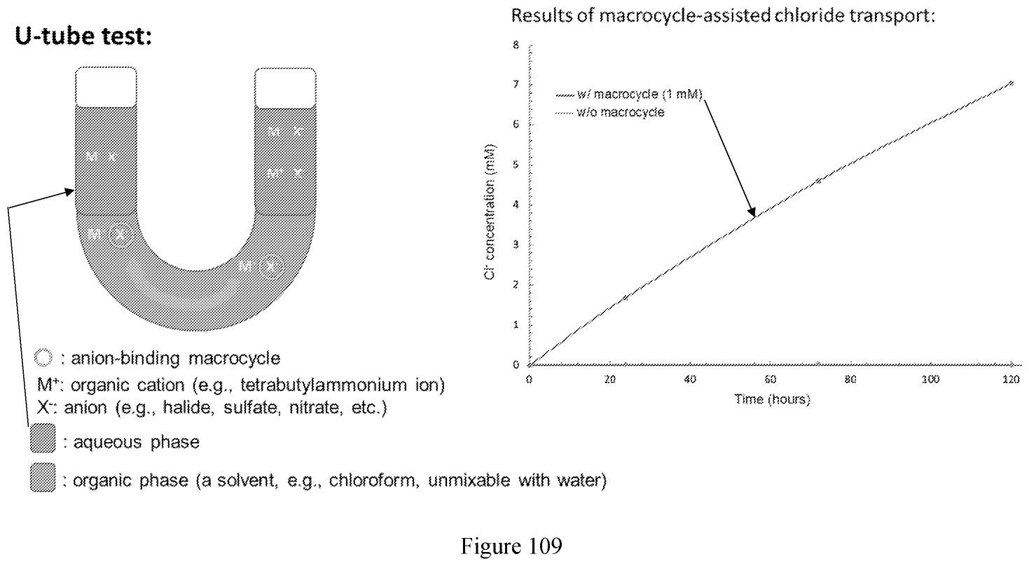

Anion binding with four amides, i.e., L1mer-L4mer, was studied in the mixed solvents of CD₃CN/CDCl₃ (1/9, v/v) by titrating an amide host with 0 to 5 equivalents of tetrabutylammonium (n-Bu₄N*) salt of the corresponding anion. The binding of chloride, bromide, iodide, and nitrate was examined with the four amides in CDCl₃ containing 10% CD₃CN. The titration data were collected by monitoring the changes in chemical shifts of the backbone amide protons and the inner aromatic protons with varying ratios of the anion guests. Job plots indicate that these linear oligoamides and their anion guests bind in a 1:1 stoichiometry. As shown in FIG. 103, for each anion, the binding affinities show a linear correlation with the length of the oligoamides, i.e., the number of amide NH groups available for H-bonding with the anion guest. For example, the Kₐ's of chloride range from the very modest 39 M⁻¹ with L1mer to over 1,000 M⁻¹ with tetramer L4mer. Different anions also showed different binding strength with the same oligoamide host. Among the halides, while the shortest L1mer binds chloride most strongly, the longer L2mer, L3mer, and L4mer all bind the bromide ion more strongly than chloride or iodide, suggesting that the bromide ion probably fits the curvature of the longer oligoamides better, which results in effective H-bonding interaction. The binding of the nitrate ion with each of the oligoamides is stronger than any of the halide ion, with the strongest binding (˜10⁴ M⁻¹) being found between L4mer and the nitrate ion. These initial results demonstrate that by varying oligoamide lengths, anion binding strength can be systematically adjusted, a very important feature for developing anion carriers capable of efficiently transporting and delivering anions across cell membranes.

Anion Binding with Macrocyclic Oligoamides: Tight Binding of Anions.

The binding of anions by macrocycle 7c was examined in CH₃CN/CHCl₃ (6/4, v/v) and in DMSO by titrating with various equivalents of the salts of chloride, iodide, nitrate, and dihydrogen phosphate, with tetrabutylammonium (n-Bu₄N*) being the counterion ion. Following the changes in the chemical shifts of backbone amide protons and “inner” aromatic protons allowed the determination of binding constants. The obtained titration data indicate that macrocycle 7c binds with these anions in a 1:1 stoichiometry.

As shown in FIG. 104, even in the rather polar solvent of CD₃CN/CDCl₃ (6/4), macrocycle 7a binds chloride, iodide, and nitrate with much larger (10⁴-10⁵ M⁻¹) Kₐ's than those observed with the linear oligoamides. The binding of acetate, bisulfate, or dihydrogen phosphate with 7c is too strong to be measurable by ¹H NMR. Even in the highly polar DMSO, the binding constants of acetate, bisulfate, or dihydrogen phosphate with 7c are still over 10³ M-1, with that of dihydrogen phosphate being over 10⁵ M⁻¹. The observed strong binding of anions by macrocycle 7c indicate that these oligoamide macrocycles could provide a new series of receptors with high affinities for anions including biologically important ones.

Expected Biological Activities.

These anion receptors, most of which show desirability solubility in most organic solvents, are expected to facilitate transmembrane anion transport and exert their biological activities such as killing cancer cells by altering the pH inside and outside cells, and treating cystic fibrosis by re-balancing chloride gradient across cell membranes.

Although the present disclosure has been described with respect to one or more particular embodiments and/or examples, it will be understood that other embodiments and/or examples of the present disclosure may be made without departing from the scope of the present disclosure.

1. A compound comprising one or more of aromatic substituents, wherein adjacent aromatic substituents are linked by at least one amide group and the compound has the following structure: 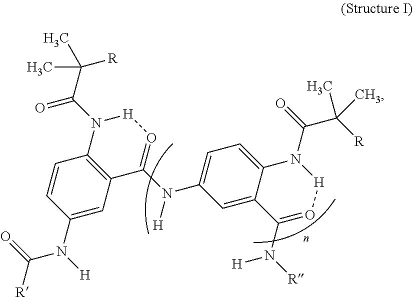
wherein n is 0 to 50
or 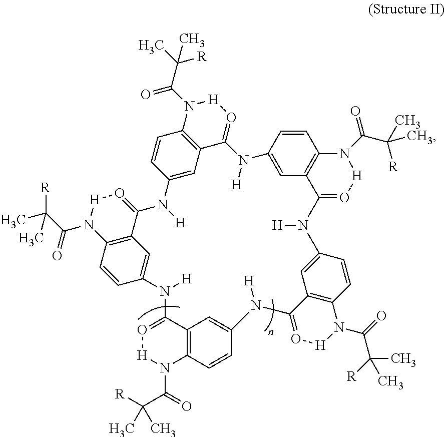
wherein n is 1 or 2, and
wherein R is independently at each occurrence chosen from linear aliphatic groups, branched aliphatic groups, fluorinated linear aliphatic groups, fluorinated branched aliphatic groups, ether groups, and oligoether groups, and
wherein R′ and R″ are independently chosen from linear aliphatic groups, branched aliphatic groups, and aryl groups.

2. The compound of claim 1, wherein the compound has Structure I and n is 0, 1, 2, 3, or 4.

3. The compound of claim 2, wherein the R′ group is methyl, ethyl, propyl, butyl, pentyl, hexyl, heptyl, octyl, or nonyl.

4. The compound of claim 2, wherein the R″ group is methyl, ethyl, propyl, butyl, pentyl, or hexyl.

5. The compound of claim 2, wherein the R group is —C(CH₃)₂CH₂O(CH₂)₇CH₃ or —C(CH₃)₂CH₂OR′″, wherein R′″ is chosen from methyl groups, ethyl groups, linear and branched propyl groups, linear and branched butyl groups, linear and branched pentyl groups, linear and branched hexyl groups, linear and branched heptyl groups, linear and branched octyl groups, and linear and branched nonyl groups.

6. The compound of claim 1, wherein the compound has Structure II.

7. The compound of claim 6, wherein n is 1.

8. The compound of claim 7, wherein the R group is 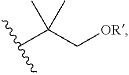
and R is —(CH₂)₇CH₃, —(CH₂CH₂O)₃CH₃, —CH₂CH═CH₂, methyl groups, ethyl groups, linear and branched propyl groups, linear and branched butyl groups, linear and branched pentyl groups, linear and branched hexyl groups, linear and branched heptyl groups, linear and branched octyl groups, linear and branched nonyl groups, linear and branched propenyl groups, linear and branched butenyl groups, linear and branched pentenyl groups, linear and branched hexenyl groups, linear and branched heptenyl groups, linear and branched octenyl groups, and linear and branched nonenyl groups.

9. The compound of claim 6, wherein n is 2.

10. A composition comprising one or more compound(s) of claim 1 and one or more hydrogen-bond acceptors and/or ions, wherein the backbone of the compound has a crescent conformation or helical conformation.

11. The composition of claim 10, wherein the hydrogen-bond acceptors and/or ions are polar guest molecules, anions, cations, or a combination thereof.

12. The composition of claim 11, wherein the anion is chosen from halide ions, nitrate ions, carbonate ions, phosphate ions, sulfate ions, oxo anions, and combinations thereof.

13. The composition of claim 10, wherein the compound that adopts a crescent or helical conformation has an interior and an exterior of the crescent or helix, and intramolecular hydrogen bonds are on the exterior of the helix and intermolecular hydrogen bonds are on the interior of the crescent or helix.

14. The composition of claim 13, wherein the interior has an inner diameter of ˜6.5 Å.

15. The composition of claim 13, wherein the interior is electrostatically positive and hydrophilic, and the exterior is hydrophobic.

16. The composition of claim 10, wherein the composition comprises a plurality of compounds that all have the same structure or a plurality of compounds wherein at least one of the compounds has a different structure.

17. A method of sequestering one or more hydrogen-bond acceptors and/or ions comprising:
contacting the one or more hydrogen-bond acceptors and/or ions with one or more compound(s) of claim 1, wherein at least a portion or all of the one or more hydrogen-bond acceptors and/or ions are sequestered by the compound(s).

18. The method of claim 17, wherein the compound(s) are disposed on a substrate.

19. The method of claim 17, wherein a sample comprises the one or more hydrogen-bond acceptors and/or ions and the sample is an organic or aqueous solution.

20. The method of claim 19, wherein the sample is a wastewater sample, an industrial water sample, a municipal water sample, or a solution in organic solvent.

21. The method of claim 17, wherein a complex is formed from the compound(s) and one or more hydrogen-bond acceptors and/or ions.

22. The method of claim 17, wherein the sequestered one or more hydrogen-bond acceptors and/or ions is/are isolated.

23. A method of treating an individual diagnosed with or suspected of having an extracellular and/or intracellular anion imbalance comprising:
administering to the individual one or more compound(s) of claim 1,
such that the extracellular and/or intracellular anion imbalance is adjusted.

24. The method of claim 23, wherein the individual has been diagnosed with cystic fibrosis.

25. The method of claim 23, wherein the physiological gradient of anion concentration in the individual is at least partially or completely restored.

26. The method of claim 23, wherein one or more symptom(s) related to the extracellular and/or intracellular anion imbalance in the individual is at least partially or completely alleviated.

27. The method of claim 23, wherein the individual is a human or a non-human animal.

28. The method of claim 23, wherein the sequestered anions(s) is/are isolated.
